(19) United States
(12) Patent Application Publication
(10) Pub. No.: US 2008/0097559 A1
(43) Pub. Date: Apr. 24, 2008

(54) Method and apparatus for carrying out the controlled heating of dermis and vascular tissue

(76) Inventors: Philip E. Eggers, Dublin, OH (US); Andrew R. Eggers, Ostrander, OH (US); Eric A. Eggers, Portland, OR (US)

Correspondence Address: MUELLER AND SMITH, LPA;MUELLER-SMITH BUILDING, 7700 RIVERS EDGE DRIVE, COLUMBUS, OH, 43235, US

(21) Appl. No.: 11/825,902

(22) Filed: Jul. 10, 2007

Related U.S. Application Data
(63) Continuation-in-part of application No. 11/583,621, filed on Oct. 19, 2006.

(51) Int. Cl. A61B 18/14

(52) U.S. Cl. 607/102

(57) ABSTRACT

Method for effecting a controlled heating of tissue within the region of dermis which employs heater implants which are configured with a thermally insulative generally flat support functioning as a thermal barrier. From the surface of this thermal barrier are supported one or more electrodes within a radiofrequency excitable circuit as well as an associated temperature sensing circuit. A model of R.F. current path flow is developed resulting in a current path index permitting a prediction of current path flow. Improved electrode excitation is developed with an intermittent R.F. excitation of electrodes shortening therapy time and improving skin protection against thermal trauma.

### CROSS-REFERENCE TO RELATED APPLICATIONS

This application is a continuation-in-part of copending U.S. patent application Ser. No. 11/583,621, filed Oct. 19, 2006.

### STATEMENT REGARDING FEDERALLY SPONSORED RESEARCH

Not applicable.

### BACKGROUND

The skin or integument is a major organ of the body present as a specialized boundary lamina, covering essentially the entire external surface of the body, except for the mucosal surfaces. It forms about 8% of the body mass with a thickness ranging from about 1.5 to about 4 mm. Structurally, the skin organ is complex and highly specialized as is evidenced by its ability to provide a barrier against microbial invasion and dehydration, regulate thermal exchange, act as a complex sensory surface, and provide for wound healing wherein the epidermis responds by regeneration and the underlying dermis responds by repair (inflammation, proliferation, and remodeling), among a variety of other essential functions.

Medical specialties have evolved with respect to the skin, classically in connection with restorative and aesthetic (plastic) surgery. Such latter endeavors typically involve human aging. The major features of the skin are essentially formed before birth and within the initial two to three decades of life are observed to not only expand in surface area but also in thickness. From about the third decade of life onward there is a gradual change in appearance and mechanical properties of the skin reflective of anatomical and biological changes related to natural aging processes of the body. Such changes include a thinning of the adipose tissue underlying the dermis, a decrease in the collagen content of the dermis, changes in the molecular collagen composition of the dermis, increases in the number of wrinkles, along with additional changes in skin composition. The dermis itself decreases in bulk, and wrinkling of senescent skin is almost entirely related to changes in the dermis. Importantly, age related changes in the number, diameter, and arrangement of collagen fibers are correlated with a decrease in the tensile strength of aging skin in the human body, and the extensibility and elasticity of skin decrease with age. Evidence indicates that intrinsically aged skin shows morphological changes that are similar in a number of features to skin aged by environmental factors, including photoaging.

See generally:
1. Gray's Anatomy, 39^{th} Edition, Churchill Livingstone, N.Y. (2005)
2. Rook's Textbook of Dermatology, 7^{th} Edition, Blackwell Science, Maiden, Mass. (2004)

A substantial population of individuals seeking to ameliorate this aging process has evolved over the decades. For instance, beginning in the late 1980s researchers who had focused primarily on treating or curing disease began studying healthy skin and ways to improve it and as a consequence, a substantial industry has evolved. By reducing and inhibiting wrinkles and minimizing the effects of ptosis (skin laxity and sagging skin) caused by the natural aging of collagen fibrils within the dermis, facial improvements have been realized with the evolution of a broad variety of corrective approaches.

Considering its structure from a microscopic standpoint, the skin is composed of two primary layers, an outer epidermis which is a keratinized stratified squamous epithelium, and the supporting dermis which is highly vascularized and provides supporting functions. In the epidermis tissue there is a continuous and progressive replacement of cells, with a mitotic layer at the base replacing cells shed at the surface. Beneath the epidermis is the dermis, a moderately dense connective tissue. The epidermis and dermis are connected by a basement membrane or basal lamina with greater thickness formed as a collagen fiber which is considered a Type I collagen having an attribute of shrinking under certain chemical or heat influences. Lastly, the dermis resides generally over a layer of contour defining subcutaneous fat. Early and some current approaches to the rejuvenation have looked to treatments directed principally to the epidermis, an approach generally referred to ablative resurfacing of the skin. Ablative resurfacing of the skin has been carried out with a variety of techniques. One approach, referred to as “dermabrasion” in effect mechanically grinds off components of the epidermis.

Mechanical dermabrasion activities reach far back in history. It is reported that about 1500 B.C. Egyptian physicians used sandpaper to smooth scars. In 1905 a motorized dermabrasion was introduced. In 1953 powered dental equipment was modified to carry out dermabrasion practices. See generally:
3. Lawrence, et al., “History of Dermabrasion” Dermatol Surg, 26:95-101 (2000).

A corresponding chemical approach is referred to by dermatologists as “chemical peel”. See generally:
4. Moy, et al., “Comparison of the Effect of Various Chemical Peeling Agents in a Mini-Pig Model” Dermatol Surg. 22:429-432 (1996).

Another approach, referred to as “laser ablative resurfacing of skin” initially employed a pulsed CO₂ laser to repair photo-damaged tissue which removed the epidermis and caused residual thermal damage within the dermis. It is reported that patients typically experienced significant side effects following this ablative skin resurfacing treatment. Avoiding side effects, non-ablative dermal remodeling was developed wherein laser treatment was combined with timed superficial skin cooling to repair tissue defects related to photo-aging. Epidermal removal or damage thus was avoided, however, the techniques have been described as having limited efficacy. More recently, fractional photothermolysis has been introduced wherein a laser is employed to fire short, low energy bursts in a matrix pattern of non-continuous points to form a rastor-like pattern. This pattern is a formation of isolated non-continuous micro-thermal wounds creating necrotic zones surrounded by zones of viable tissue. See generally:
5. Manstein, et al., “Fractional Photothermolysis: A New. Concept for Cutaneous Remodeling Using Microscopic Patterns of Thermal Injury” Lasers in Surgery and Medicine, 34:426-438 (2004).

These ablative techniques (some investigators consider fractional photothermolysis as a separate approach) are associated with drawbacks. For instance, the resultant insult to the skin may require 4-6 months or more of healing to evolve newer looking skin. That newer looking skin will not necessarily exhibit the same shade or coloration as its original counterpart. In general, there is no modification of the dermis in terms of a treatment for ptosis or skin laxity through collagen shrinkage.

To treat patients for skin laxity, some investigators have looked to procedures other than plastic surgery. Techniques for induced collagen shrinkage at the dermis have been developed. Such shrinkage qualities of collagen have been known and used for hundreds of years, the most classic example being the shrinking of heads by South American headhunters. Commencing in the early 1900s shrinking of collagen has been used as a quantitative measure of tanning with respect to leather and in the evaluation of glues. See:
6. Rasmussen, et al., “Isotonic and Isometric Thermal Contraction of Human Dermis I. Technic and Controlled Study”, J. Invest. Derm. 43:333-9 (1964).

Dermis has been heated through the epidermis utilizing laser technology as well as intense pulsed light exhibiting various light spectra or single wavelength. The procedure involves spraying a burst of coolant upon the skin such as refrigerated air, whereupon a burst of photons penetrates the epidermis and delivers energy into the dermis.

Treatment for skin laxity by causing a shrinkage of collagen within the dermis generally involves a heating of the dermis to a temperature of about 60° C. to 70° C. over a designed treatment interval. Heat induced shrinkage has been observed in a course of laser dermabrasion procedures. However, the resultant energy deposition within the epidermis has caused the surface of the skin to be ablated (i.e., burned off the surface of the underlying dermis) exposing the patient to painful recovery and extended healing periods which can be as long as 6-12 months. See the following publication:
7. Fitzpatrick, et al., “Collagen Tightening Induced by Carbon Dioxide Laser Versus Erbium: YAG Laser” Lasers in Surgery and Medicine 27: 395-403 (2000).

Dermal heating in consequence of the controlled application of energy in the form of light or radiofrequency electrical current through the epidermis and into the dermis has been introduced. To avoid injury to the epidermis, cooling methods have been employed to simultaneously cool the epidermis while transmitting energy through it. In general, these approaches have resulted in uncontrolled, non-uniform and often inadequate heating of the dermis layer resulting in either under-heating (insufficient collagen shrinkage) or over heating (thermal injury) to the subcutaneous fat layer and/or weakening of collagen fibrils due to over-shrinkage. See the following publication:
8. Fitzpatrick, et al., “Multicenter Study of Noninvasive Radiofrequency for Periorbital Tissue Tightening”, Lasers in Surgery in Medicine, 33:232-242 (2003).

The RF approach described in publication 8 above is further described in U.S. Pat. Nos. 6,241,753; 6,311,090; 6,381,498; and 6,405,090. Such procedure involves the use of an electrode capacitively coupled to the skin surface which causes radiofrequency current to flow through the skin in monopolar fashion to a much larger return electrode located remotely upon the skin surface of the patient. Note that the electrodes are positioned against skin surface and not beneath it. The radiofrequency current density caused to flow through the skin is selected to be sufficiently high to cause resistance heating within the tissue and reach temperatures sufficiently high to cause collagen shrinkage and thermal injury, the latter result stimulating beneficial growth of new collagen, a reaction generally referred to as “neocollagenesis”.

Uniform heating of the dermal layer generally is called for in the presence of an assurance that the underlying fat layer is not adversely affected while minimal injury to the epidermis is achieved. A discussion of the outcome and complications of the noted non-ablative mono-polar radiofrequency treatment is provided in the following publication:
9. Abraham, et al., “Current Concepts in Nonablative Radiofrequency Rejuvenation of the Lower Face and Neck” Facial Plastic Surgery, Vol. 21 No. 1 (2005).

In the late 1990s, Sulamanidze developed a mechanical technique for correcting skin laxity. With this approach one or more barbed non-resorbable sutures are threaded under the skin with an elongate needle. The result is retention of the skin in a contracted state and, over an interval of time, the adjacent tissue will ingrow around the sutures to stabilize the facial correction. See the following publications:
10. Sulamanidze, et al., “Removal of Facial Soft Tissue Ptosis With Special Threads”, Dermatol Surg., 28:367-371 (2002).
11. Lycka, et al., “The Emerging Technique of the Antiptosis Subdermal Suspension Thread”, Dermatol Surg., 30:41-44 (2004).

Eggers, et al., in application for U.S. patent Ser. No. 11/298,420 entitled “Aesthetic Thermal Sculpting of Skin”, filed Dec. 9, 2005 describes a technique for directly applying heat energy to dermis with one or more thermal implants providing controlled shrinkage thereof. Importantly, while this heating procedure is underway, the subcutaneous fat layer is protected by a polymeric thermal barrier. In one arrangement this barrier implant is thin and elongate and supports a flexible resistive heating circuit, the metal heating components of which are in thermal exchange contact with dermis. Temperature output of this resistive heating circuit is intermittently monitored and controlled by measurement of a monitor value of resistance. For instance, resistive heating is carried out for about a one hundred millisecond interval interspersed with one millisecond resistance measurement intervals. Treatment intervals experienced with this system and technique will appear to obtain significant collagen shrinkage within about ten minutes to about fifteen minutes. During the procedure, the epidermis is cooled by blown air.

Eggers et al., in application for U.S. patent Ser. No. 11/583,555 entitled “Method and Apparatus for Carrying Out the Controlled Heating of Tissue in the Region of Dermis”, filed Oct. 19, 2006 describes an improved utilization of such barrier implants wherein a slight pressure or tamponade is applied over the skin region during treatment to an extent effective to maintain substantially continuous conduction heat transfer between tissue in the region of the dermis and the implant heater segments. One result is an important lessening of required treatment time.

Eggers et al., in application U.S. patent Ser. No. 11/583,621 entitled “Method and Apparatus for Carrying Out the Controlled Heating of Tissue in the Region of Dermis”, filed Oct. 19, 2006 describes a bipolar radiofrequency implementation of the barrier implants wherein a continuous power modulating ramping up of power and electrode temperature occurs until a threshold level is reached. Once that level is reached, the continuous power is reduced for a soak interval. Treatment time is advantageously short with the bipolar R.F. approach.

Particularly where barrier implants are implemented using bipolar R.F. energy, protection of the epidermis from thermal damage has remained a concern. Cooling of the skin surface is called for at least during treatment. Such cooling must be sufficient to protect the epidermis while still permitting an effective heating of dermis to achieve proper collagen shrinkage.

Some of the procedures described above may be carried out using local anesthesia. Local anesthetic agents may be, for example, weakly basic tertiary amines, which are manufactured as chloride salts. The molecules are amphipathic and have the function of the agents and their pharmacokinetic behavior can be explained by the structure of the molecule. Such local anesthetics have a lipophilic side; a hydrophilic-ionic side; an intermediate chain, and, within the connecting chain, a bond. That bond determines the chemical classification of the agents into esters and amides. It also determines the pathway for metabolism. While there are a variety of techniques for administering local anesthesia, in general, it may be administered for infiltration, activity or as a nerve block. In each approach, the active anesthetic drug is administered for the purpose of intentionally interrupting neural function and thereby providing pain relief.

A variety of local anesthetics have been developed, the first agent for this purpose being cocaine which was introduced at the end of the nineteenth century. Lidocaine is the first amide local anesthetic and the local anesthetic agent with the most versatility and thus popularity. It has intermediate potency, toxicity, onset, and duration, and it can be used for virtually any local anesthetic application. Because of its widespread use, more knowledge is available about metabolic pathways than any other agent. Similarly, toxicity is well known.

Vasoconstrictors have been employed with the local anesthetics. In this regard, epinephrine has been added to local anesthetic solutions for a variety of reasons throughout most of the twentieth century to alter the outcome of conduction blockade. Its use in conjunction with infiltration anesthesia consistently results in lower plasma levels of the agent. See generally:
12. “Clinical Pharmacology of Local Anesthetics” by Tetzlaff, J. E., Butterworth-Heinemann, Woburn, Mass. (2000).

To minimize the possibility of irreversible nerve injury in the course of using local anesthetics, the drugs necessarily are diluted. By way of example, the commonly used anesthetic drug is injected using concentrations typically in the range of 0.4% to 2.0% (weight percent). The diluent contains 0.9% sodium chloride. Such isotonic saline is used as the diluent due to the fact that its osmolarity at normal body temperature is 286 milliOsmols/liter which is close to that of cellular fluids and plasma which have a osmolarity of 310 milliOsmols/liter. As a result, the osmotic pressure developed across the semipermeable cell membranes is minimal when isotonic saline is injected. Consequently, there is no injury to the tissue's cells surrounded by this diluent since there is no significant gradient which can cause fluids to either enter or leave the cells surrounded by the diluent. It is generally accepted that diluents having an osmolarity in the range of 240 to 340 milliOsmols/liter are isotonic solutions and therefore can be safely injected.

A variety of aberrant vascular formations, i.e. angiomas, hemangiomas vascular malformations and other vascular anomalies, are present near the surface of the skin, such that these aberrant vascular formations display a visual or structural alteration of the appearance of the skin. Aberrant vascular formations may occur in arterial, venuous, or lymphatic tissues. Mulliken and Glowacki distinguished vascular anomalies (lesions) into two major categories, angiomas and vascular malformations. Vascular malformations are further subdivided and characterized as arterial, venuous, lymphatic, capillary and mixed (e.g., arterio-capillary-venuous). Jackson, et al., along with the ISSVA have provided further categorization of vascular lesions as being classified as either vascular tumors (i.e. angiomas, a term currently disfavored by the ISSVA, but utilized in the literature) or vascular malformations. See
13. Jackson, et al., “Hemangiomas, vascular malformations and lymphovenous malformations: classifications and methods of treatment.” Plat. Recon. Surg., 91: 1216-30 (1993).
14. “ISSVA Classification” (of vascular malformations) excerpt from Color Atlas of Vascular Tumors and Vascular Malformations, by O. Enjolras, M. Wassef and R. Chapot Cambridge University Press (2007).

Vascular tumors or angiomas are known as one type of aberrant vascular formation that are presented on the surface of the skin. Angiomas are an aberrantly or hyperplastically proliferating vascular tissue and include, for example, benign infantile hemangiomas; congenital hemangiomas; tufted angioma, with or without Kasabach-Merritt syndrome; Kaposiform hemangioendothelioma; spindle cell hemangioendothelioma; other rare hemangioendotheliomas, including epithelioid, composite, retiform, polymorphous, Dabska Tumor, and lymphangioendotheliomatosis; and dermatologic acquired vascular tumors, including pyogenic granuloma, targetoid hemangioma, glomeruloid hemangioma and microvenular heangioma. Hemangiomas are localized tumors of blood vessels, and may be generally classified, particularly with respect to infantile hemangiomas, as either proliferating (progressive growth), involuting (slowing rate of growth or regressing), or involuted (stable, with no further regression). Hemangiomas appear in approximately 10% of Caucasian infants, with complete regression occurring by age 7 in 70% of children. See:
15. Takahashi, et al., J. Clin. Invest. 93: 2357-2364. (June 1994).
   Angiomas exhibit increased endothelial cell turnover, and the proliferating stage is characterized by the expression of Type IV collagenase, and growth factors such as vascular endothelial growth factor (VEGF) and basic fibroblast growth factor (bFGF). Lymphangiomas are tumors of the lymphatic system and are usually benign and congenital, with approximately 75% occurring in the cervical region.

Jackson, et al., and the ISSVA classification subdivide a variety of nonproliferative vascular malformations, e.g., vascularity with quiescent endothelium and considered to be localized defects of vascular morphogenesis, and include such low flow vascular malformations such as, for instance, capillary malformations (CM), including Port-Wine stain (PWS), nevus flammeus, telangiectasia, and angiokeratoma; venuous malformation (VM), including, common sporadic VM, Bean syndrome, familial cutaneous and mucosal VM, glomuvenous malformation (GVM) and Mafucci syndrome; and lymphatic malformation (LM), fast-flow vascular malformations such as, for instance, arterial malformation, (AM); arteriovenous fistula (AVM) and arteriovenous malformation (AVM), and complex-combined vascular malformations such as, for instance CVM, CLM, LVM, CLVM, AVM-LM and CM-AVM.

Laser induced interstitial thermotherapy has been applied to the treatment of vascular anomalies, with effects differing from malignant cell necrosis, irreversible tissue damage or carbonization, depending on the maximum temperature to which the treated tissue has been heated. Successful outcomes utilizing tissue heating are highly dependent on effective monitoring of the temperature increase induced by localized heating, especially when vital anatomic structures are located in close proximity to treated tissues. Carbonization of tissue is difficult to completely avoid when utilizing laser induced interstitial thermotherapy, and once a carbonized tissue volume is created, this carbonized volume is not generally mobilized by metabolic processes, and can lead to deleterious side effects such as abscess formation. Thus, when utilizing thermotherapy techniques, reliable and accurate quantitative tissue temperature monitoring of great importance to avoid damage to healthy or untargeted tissue and organs, and to avoid induction of structures that might lead to deleterious side effects. One relatively unwieldy system available for tissue temperature monitoring is real-time magnetic resonance imaging. See, e.g.:
16. Eyrich et al., “Temperature mapping of magnetic resonance guided laser interstitial therapy (LITT) in lymphangiomas of the head and neck.” Lasers in Surgery and Medicine 26: 467-476 (2000).

Established treatment modalities for vascular anomalies include surgery, intralesional sclerotherapy and topical or interstitial heating using lasers, for instance Nd:YAG lasers. In most cases resection of extensive vascular anomalies in their entirety is not feasible, and unresected portions of a vascular anomaly may rapidly re-expand. Resection of a large lesions is hazardous due to risk of uncontrollable bleeding and mutilation of superficial surfaces due to extensive resection. Additional side effects of the above identified treatment modalities that suffer from inadequate control of tissue disruption include parethesia, tirsmus and local motoric plegia.

The dermis is the primary situs of congenital birthmarks generally deemed to be aberrant vascular formations or vascular lesions as capillary malformations, including those historically referred to as nevus flammeus and “Port-Wine Stains” (PWS). Ranging in coloration from pink to purple, these non-proliferative lesions are characterized histologically by ecstatic vessels of capillary or venular type within the papillary and reticular dermis and are considered as a type of vascular malformation. The macular lesions are relatively rare, occurring in about 0.3% of newborns and generally appear on the skin of the head and neck within the distribution of the trigeminal (fifth cranial) nerve. They persist throughout life and may become raised, nodular, or darken with age. Their depth has been measured utilizing pulsed photothermal radiometry (PPTR) and ranges from about 200 μm to greater than 1000 μm.

See the following publication:
17. Bincheng, et al., Accurate Measurement of Blood Vessel Depth in Port Wine Stain Human Skin in vivo Using “Photothermal Radiometry”, J. Biomed. Opt. (5), 961-966 (September/October 2004).

Fading or lightening the PWS lesions has been carried out with lasers with somewhat mixed results. For instance, they have been treated with pulsed dye lasers (PDL) at 585 mm wavelength with a 0.45 ms pulse length and 5 mm diameter spot size. Cryogenic bursts have been used with the pulsing for epidermal protection. Generally, the extent of lightening achieved is evaluated six to eight weeks following laser treatment. Such evaluation assigns the color of adjacent normal skin as 100% lightening and a post clearance, evaluation of lesions will consider more than 75% lightening as good.

See the following publication:
18. Fiskerstrand, et al., “Laser Treatment of Port Wine Stains: Thereaupetic Outcome in Relation to Morphological Parameters” Brit. J. of Derm., 134, 1039-1043, (1996).

Capillary malformation lesions have been classified, for instance, utilizing video microscopy, three patterns of vascular ectasia being established; type 1, ectasia of the vertical loops of the papillary plexus; type 2, ectasia of the deeper, horizontal vessels in the papillary plexus; and type 3, mixed pattern with varying degrees of vertical and horizontal vascular ectasia. In general, due to the limited depth of laser therapy, only type 1 lesions are apt to respond to such therapy.

Port wine stains also are classified in accordance with their degree of vascular ectasia, four grades thereof being recognized, Grades I to IV. Grade 1 lesions are the earliest lesions and thus have the smallest vessels (50-80 um in diameter). Using ×6 magnification and transillumination, individual vessels can only just be discerned and appear like grains of sand. Clinically, these lesions are light or dark pink macules. Grade II lesions are more advanced (vessel diameter=80-120 um). Individual vessels are clearly visible to the naked eye, especially in less dense areas. They are thus clearly distinguishable macules. Grade III lesions are more ecstatic (120-150 um). By this stage, the space between the vessels has been replaced by the dilated vessels. Individual vessels may still be visible on the edges of the lesion or in a less dense lesion, but by and large individual vessels are no longer visible. The lesion is usually thick, purple, and palpable. Eventually dilated vessels will coalesce to form nodules, otherwise known as cobblestones. Grade IV represents the largest vessels. The main purpose of these classifications has been to assign a grade for ease in communication between practitioners and for ease of determination of the appropriate laser treatment settings.

See the following publication:
19. Mihm, Jr., et al, “Science, Math and Medicine—Working Together to Understand the Diagnosis, Classification and Treatment of Port-Wine Stains”, a paper presented in Mt. Tremblant, Quebec, Canada, 2004, Controversies and Conversations in Cutaneous Laser Surgery—An Advanced Symposium.

### BRIEF SUMMARY OF THE INVENTION

The present disclosure is addressed to embodiments of methods for effecting a controlled heating of tissue within the region of the dermis of skin. Heater implants or wands are employed which are configured with a thermally and electrically insulative flat support functioning as a thermal barrier as well as to support a flexible circuit assembly carrying radiofrequency driven electrodes and associated temperature sensors present as resistor segments.

Research is described in which these implants are employed in bipolar fashion in conjunction with both ex vivo and in vivo animal studies. Histopathology analysis of resultant specimens was carried out and evaluated to discern the nature of R.F. current flow induced by electrodes located at the interface between dermis and next subcutaneous fat layer. A small number of the analyzed specimens indicated a penetration of aberrant current into muscle underlying the fat layer. Electrical characteristics for dermis, subcutaneous fat, and muscle were compiled and a model formulated based upon parameters associated with R.F. bipolar heating employing the noted wands. This model, referred to as a current path index (CPI) is used to predict R.F. current flux performance. To improve such performance a topical use of an agent effective to enhance the electrical conductivity of dermis is described.

Thermal performance of the paired bipolar R.F. excited wands was evaluated using field test cells. These experiments revealed that the thermal buildup was uniform and gradual commencing at the midpoint between paired bipolar implants and gradually extending thereover. The use of adjuvants is described which are administered generally to dermis and are effective to lower the thermal transition temperature for carrying out the shrinkage of dermis or a component of dermis.

Thermal studies further developed a revised electrode R.F. excitation approach wherein the electrodes are intermittently energized to establish on-intervals spaced apart in time with off-intervals. The on-intervals are developed with a high level power input. The result is to advantageously lessen therapy time while permitting an improved control over skin surface temperature.

Accordingly, another feature of this disclosure is a method for effecting a heating of tissue within the region of the dermis of skin comprising the steps:

(a) determining a skin region for treatment;

(b) providing one or more implants each having one or more R.F. excitable electrodes;

(c) determining one or more heating channel locations along the skin region;

(d) locating each heater implant along a heating channel generally at the interface between dermis and next adjacent subcutaneous tissue wherein the one or more electrodes are contactable with dermis;

(e) selecting a temperature threshold level for the one or more electrodes;

(f) effecting radiofrequency power energization of said one or more electrodes wherein said energization is carried out during power-on intervals spaced apart in time by power-off intervals at least to substantially maintain said temperature threshold level; and

(g) simultaneously controlling the temperature of the surface of the skin within the region to an extent effective to protect epidermis from thermal injury while permitting the derivation of effected treatment temperature at the region of the dermis.

Other objects of the disclosure of embodiments will, in part, be obvious and will, in part, appear hereinafter.

The instant presentation, accordingly, comprises embodiments of the apparatus and method possessing the construction, combination of elements, arrangement of parts and steps which are exemplified in the following detailed disclosure.

For a fuller understanding of the nature and objects herein involved, reference should be made to the following detailed description taken in connection with the accompanying drawings.

### BRIEF DESCRIPTION OF THE DRAWINGS

FIG. 1 is a diagram of the structure of the extra-cellular matrix of dermis tissue;

FIG. 2 is a family of curves relating linear shrinkage of dermis of time and temperature;

FIG. 3 is a schema representing the organization of skin;

FIG. 4 is a perspective view of an experimental implant combining a thermal barrier, electrode and thermocouple;

FIG. 5 is a sectional view taken through the plane 5-5 the experimental implant shown in FIG. 4;

FIG. 6 is a perspective exploded view showing the structuring of a wand employed with the invention;

FIG. 7 is a sectional view taken through the plane 7-7 shown in FIG. 8;

FIG. 8 is a perspective view of an assembled wand employed with the present method and apparatus;

FIG. 9 is a sectional view taken through the plane 9-9 shown in FIG. 8 and further showing a portion of a polymeric cable connector;

FIG. 10 is perspective view of single implant with spaced apart bipolar electrodes;

FIG. 11 is a schematic sectional view showing a current flux path developed with the implant of FIG. 10;

FIG. 12 is an enlarged broken away top view of the forward region of the implant of FIG. 8;

FIG. 13 is an enlarged top view showing the lead components located at the trailing end of the implant of FIG. 8;

FIG. 14 is an enlarged broken away view of the inward side of the substrate component of the implant of FIG. 8;

FIG. 15 is an enlarged view of the trailing end of the substrate shown in FIG. 14;

FIG. 16 is a bottom view of an introducer instrument;

FIG. 17 is a side view of the instrument of FIG. 16;

FIG. 18 is a schematic sectional view of skin showing current flow paths between spaced apart wands and a conformal liquid containing heat sink;

FIG. 19 is a schematic curve set relating electrode temperature and times with respect to a controlled ramp-up of power to a setpoint temperature followed by a thermal soak interval at a reduced constant power, two setpoint temperatures being illustrated;

FIG. 20 is a schematic sectional view of skin, subcutaneous fat and muscle in conjunction with spaced-apart bipolar performing wands;

FIG. 21 is a schematic representation of an intermittent mode form of radiofrequency electrode excitation wherein power-on application intervals are spaced in time by power-off intervals;

FIG. 22 is a top view of an electric field test cell;

FIG. 23 is a sectional view taken through the plane 23-23 in FIG. 22;

FIG. 24 is a top view of another electric field test cell wherein three, four-electrode wands were employed;

FIG. 25 is a block diagram of components within a control console;

FIG. 26 is a schematic representation of the flexible circuit assemblies for three implants or wands;

FIG. 27 is a schematic sectional view of skin showing spaced apart bipolar wands and indicating heat transfer;

FIG. 28 is a plot showing three curves relating maximum temperature rise at the epidermis/dermis boundary with respect to an area of heat conduction which is 15 mm×18 mm;

FIG. 29 is a plot of three curves relating three surface temperatures of skin, assuming an epidermis thickness of 0.15 mm and relating heat conducted from dermis through epidermis to skin surface in watts;

FIG. 30 provides a plot of three curves with respect to three epidermis surface temperatures and relating the maximum temperature rise at the epidermis/dermis boundary with respect to heat conducted from dermis through epidermis to skin surface and assuming epidermis thickness of 0.20 mm;

FIG. 31 is a plot of three curves similar to FIGS. 29 and 30 but assuming an epidermis thickness of 0.08 mm;

FIG. 32 is a scatter diagram relating a depth of acute coagulative damage as a function of current path index;

FIGS. 33A-33J combine as labeled thereon to provide a flowchart describing procedures according to the present method and apparatus with respect to shrinkage of dermis; and

FIGS. 34A-34H combine as labeled thereon to provide a flowchart describing a method for the treatment of port wine stain.

### DETAILED DESCRIPTION OF THE INVENTION

The discourse to follow will reveal that the system, method and implants described were evolved over a sequence of animal (pig) experiments, both ex vivo and in vivo. In this regard, certain of the experiments and their results are described to, in effect, set forth a form of invention history giving an insight into the reasoning under which the embodiments developed.

The arrangement of the physical structure of the dermis is derived in large part from the structure of the extracellular matrix surrounding the cells of the dermis. The term extracellular matrix (ECM) refers collectively to those components of a tissue such as the dermis that lie outside the plasma membranes of living cells, and it comprises an interconnected system of insoluble protein fibers, cross-linking adhesive glycoproteins and soluble complexes of carbohydrates and carbohydrates covalently linked to proteins (e.g. proteoglycans). A basement membrane lies at the boundary of the dermis and epidermis, and is structurally linked to the extracellular matrix of the dermis and underlying hypodermis. Thus the extracellular matrix of the dermis distributes mechanical forces from the epidermis and dermis to the underlying tissue.

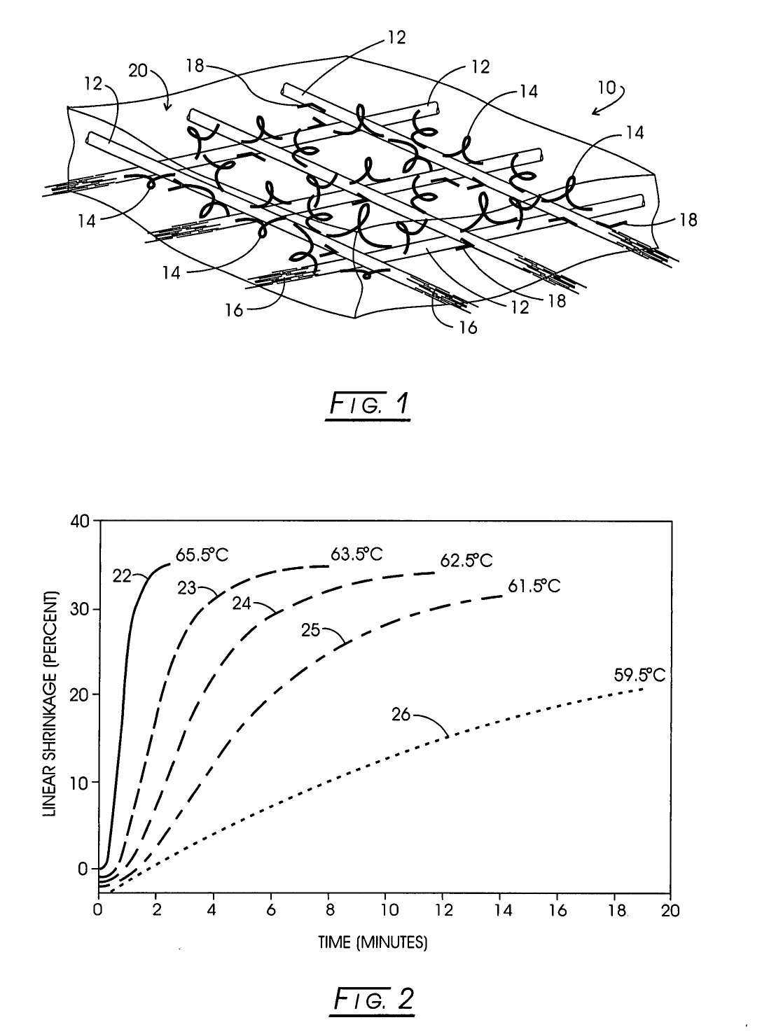

Looking to FIG. 1, a schematic representation of a region of the extracellular matrix of the dermis is represented generally at 10. The insoluble fibers include collagen fibers at 12, most commonly collagen Type I, and elastin at 14. The fundamental structural unit of collagen is a long, thin protein (300 nm×15 nm) composed of three subunits coiled around one another to form the characteristic right-handed collagen triple helix. Collagen is formed within the cell as procollagen, wherein the three subunits are covalently cross-linked to one another by disulphide bonds, and upon secretion are further processed into tropocollagen. The basic tropocollagen structure consists of three polypeptide chains coiled around each other in which the individual collagen molecules are held in an extended conformation. The extended conformation of a tropocollagen molecule is maintained by molecular forces including hydrogen bonds, ionic interactions, hydrophobicity, salt links and covalent cross-links. Tropocollagen molecules are assembled in a parallel staggered orientation into collagen fibrils at 16, each containing a large number of tropocollagens, held in relative position by the above listed molecular forces and by cross-links between hydrolysine residues of overlapping tropocollagen molecules. Certain aspects of collagen stabilization are enzyme mediated, for example by Cu-dependent lysyl oxidase. Collagen fibrils are typically of about 50 nm in diameter. Type I collagen fibrils have substantial tensile strength, greater on a weight basis than that of steel, such that the collagen fibril can be stretched without breaking. Collagen fibrils are further aggregated into more massive collagen fibers, as previously shown at 12. The aggregation of collagen fibers involves a variety of molecular interactions, such that it appears that collagen fibers may vary in density based on the particular interactions present when formed. Elastin, in contrast to collagen, does not form such massive aggregated fibers, may be thought of as adopting a looping conformation (as shown at 14) and stretch more easily with nearly perfect recoil after stretching.

The extracellular matrix (ECM) as at 10 lies outside the plasma membrane, between the cells forming skin tissue. ECM components including tropocollagen, are primarily synthesized inside the cells and then secreted into the ECM through the plasma membrane. The overall structure and anatomy of the skin, and in particular the dermis, are determined by the close interaction between the cells and ECM. Referring again to FIG. 1, only a few of the many and diverse components of the ECM are shown. In addition to collagen fibers 12 and elastin 14 are a large number of other components that serve to crosslink or cement these named components to themselves and to other components of the ECM. Such crosslinking components are represented as at 18, and may be of protein, glycoprotein and or carbohydrate composition, for example. The cross-linked collagen fibers shown in FIG. 1 are embedded in a layer of highly hydrated material, including a diverse variety of modified carbohydrates, including particularly the large carbohydrate hyaluronic acid (hyaluronan) and chondroitin sulphate. Hyaluronan is a very large, hydrated, non-sulphated mucopolysaccaride that forms highly viscous fluids. Chondroitin sulphate is a glycosaminoglycan component of the ECM. Accordingly, the volume of the ECM as represented generally at 20 is filled with a flexible gel with a hydrated hyaluronan component that surrounds and supports the other structural components such as collagen and elastin. Thus the structural form of the dermis may be thought to be composed of collagen, providing tensile strength, with the collagen being held in place within a matrix of hyaluronan, which resists compression. Underlying this structure are the living cells of the dermis, which in response to stimuli (such as wounds or stress, for instance) can be induced to secrete additional components, synthesize new collagen (i.e. neocollagenesis), and otherwise alter the structural form of the ECM and the skin itself. The structure of the collagen reinforced connective tissues should not be considered entirely static, but rather that the net accumulation of collagen connective tissues is an equilibrium between synthesis and degradation of the components of the collagen reinforced connective tissues. Similarly, the other components of the ECM are modulated in response to environmental stimuli.

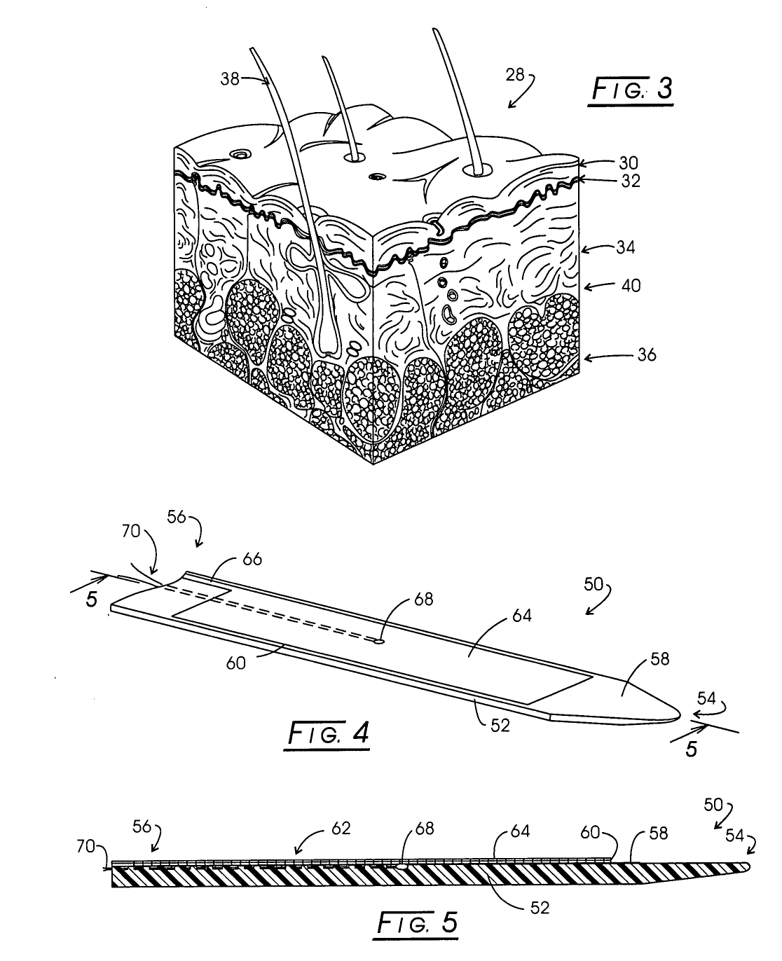

As noted earlier previous researchers have shown that collagen fibers can be induced to shrink in overall length by application of heat. Experimental studies have reported that collagen shrinkage is, in fact, dependent upon the thermal dose (i.e., combination of time and temperature) in a quantifiable manner. (See publication 16, infra). Looking to FIG. 2, a plot of linear collagen shrinkage versus time for various constant temperatures is revealed in association with plots or lines 22-26. For instance, at line 24, linear shrinkage is seen to be about 30% for a temperature of 62.5° C. held for a ten minute duration. Curve 24 may be compared with curve 22 where shrinkage of about 36% is achieved in very short order where the temperature is retained at 65.5° C. Correspondingly, curve 26 shows a temperature of 59.5° C. and a very slow rate of shrinkage, higher levels thereof not being reached. Clinicians generally would prefer a shrinkage level on the order of 10% to 20% in dealing with skin laxity.

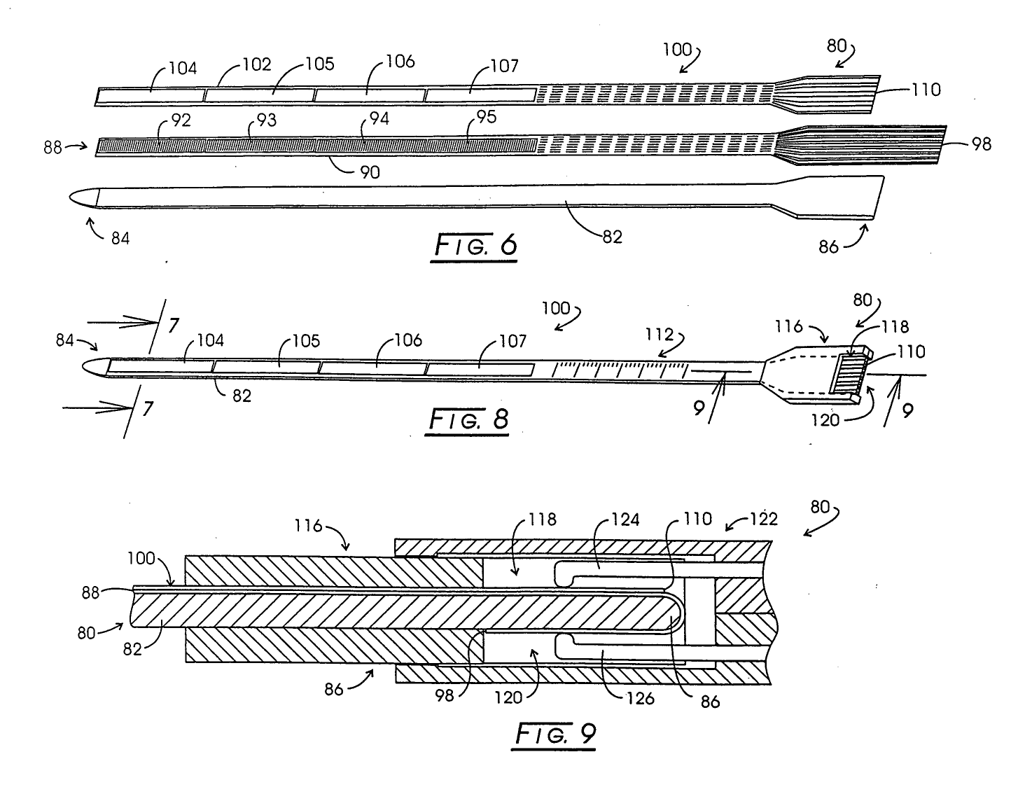

FIG. 3 reveals a schema representing the organization of skin. Shown generally at 28, the illustrated skin structure is one of two major skin classes of structure and functional properties representing thin, hairy (hirsute) skin which constitutes the great majority of the body's covering. This is as opposed to thick hairless (glabrous) skin from the surfaces of palms of hands, soles of feet and the like. In the figure, the outer epidermis layer 30 is shown generally having inwardly disposed rete ridges or pegs 32 and extending over the dermis layer represented generally at 34. Dermis 34, in turn, completes the integument and is situated over an adjacent subcutaneous tissue layer represented generally at 36. Those involved in the instant subject matter typically refer to this adjacent subcutaneous layer 36 which has a substantial adipose tissue component as a “fat layer” or “fatty layer,” and this next adjacent subcutaneous tissue layer is also called the “hypodermis” by some artisans. The figure also reveals a hair follicle and an associated shaft of hair 38. Not shown in FIG. 3 are a number of other components, including the cellular structure of the dermis, and the vascular tissues supplying the vascularized dermis and its overlying epidermis.

Epidermis 30 in general comprises an outer or surface layer, the stratum corneum, composed of flattened, cornified non-nucleated cells. This surface layer overlays a granular layer, stratum granulosum, composed of flattened granular cells which, in turn, overlays a spinous layer, stratum spinosum, composed of flattened polyhedral cells with short processes or spines and, finally, a basal layer, stratum basale, composed columnar cells arranged perpendicularly. For the type skin 28, the epidermis will exhibit a thickness from about 0.07 to 0.20 mm. Heating implants or wands described herein will be seen to be contactable with the dermis 34 at a location shown generally at 40 representing the interface between dermis 34 and next adjacent subcutaneous tissue or fat layer 36. The dermis in general comprises a papillary layer, subadjacent to the epidermis, and supplying mechanical support and metabolic maintenance of the overlying epidermis. The papillary layer of the dermis is shaped into a number of papillae that interdigitate with the basal layer of the epidermis, with the cells being densely interwoven with collagen fibers. The reticular layer of the dermis merges from the papillary layer, and possesses bundles of interlacing collagen fibers (as shown in FIG. 1) that are typically thicker than those in the papillary layer, forming a strong, deformable, three dimensional lattice around the cells of the reticular dermis. Generally, the dermis is highly vascularized, especially as compared to the avascular epidermis. The dermis layer 34 will exhibit a thickness of from about 1.0 mm to about 3.0 mm to 4.0 mm.

For the purposes of the application, “intradermal” is defined as within the dermis layer of the skin itself. “Subcutaneous” has the common definition of being below the skin, i.e. near, but below the epidermis and dermis layers. “Subdermal” is defined as a location immediately interior to, or below the dermis, at the interface 40 between the dermis and the next adjacent subcutaneous layer. “Hypodermal” is defined literally as under the skin, and refers to an area of the body below the dermis, within the hypodermis, and is usually not considered to include the subadjacent muscle tissue. “Peridermal” is defined as in the general area of the dermis, whether intradermal, subdermal or hypodermal. Transdermal is defined in the art as “entering through the dermis or skin, as in administration of a drug applied to the skin in ointment or patch form,” i.e. transcutaneous. A topical administration as used herein is given its typical meaning of application at skin surface.

As noted, the thickness of the epidermis and dermis vary within a range of only a few millimeters. Thus subcutaneous adipose tissue is responsible in large part for the overall contours of the skin surface, and the appearance of the individual patient's facial features, for instance. The size of the adipose cells may vary substantially, depending on the amount of fat stored within the cells, and the volume of the adipose tissue of the hypodermis is a function of cell size rather than the number of cells. The cells of the subcutaneous adipose tissue, however, have only limited regenerative capability, such that once killed or removed, these cells are not typically replaced. Any treatment modality seeking to employ heat to shrink the collagen of the ECM of the skin, must account for the risk associated with damaging or destroying the subcutaneous adipose layer, with any such damage representing a large risk of negative aesthetic effects on the facial features of a patient.

In general, the structural features of the dermis are determined by a matrix of collagen fibers forming what is sometimes referred to as a “scaffold.” This scaffold, or matrix plays an important role in the treatment of skin laxity in that once shrunk, it must retain it's position or tensile strength long enough for new collagen evolved in the healing process to infiltrate the matrix. That process is referred to as “neocollagenesis.” Immediately after the collagen scaffold is heated and shrunk portions of it are no longer vital because of having been exposed to a temperature evoking an irreversible denaturation. Where the scaffold retains adequate structural integrity in opposition to forces that would tend to pull it back to its original shape, a healing process requiring about four months will advantageously occur. During this period of time, neocollagenesis is occurring, along with the deposition and cross linking of a variety of other components of the ECM. In certain situations, collagen is susceptible to degradation by collagenase, whether native or exogenous.

Studies have been carried out wherein the mechanical properties of collagen as heated were measured as a function of the amount of shrinkage induced. The results of one study indicated that when the amount of linear shrinkage exceeds about 20%, the tensile strength of the collagen matrix or scaffold is reduced to a level that the contraction may not be maintained in the presence of other natural restorative forces present in tissue. Hence, with excessive shrinkage, the weakened collagen fibrils return from their now temporary contracted state to their original extended state, thereby eliminating any aesthetic benefit of attempted collagen shrinkage. The current opinion of some investigators is that shrinkage should not exceed about 25%.

One publication reporting upon such studies describes a seven-parameter logistic equation (sigmoidal function) modeling experimental data for shrinkage, S, in percent as a function of time, t, in minutes and temperature, T, in degrees centigrade. That equation may be expressed as follows:

$$S ( t , T ) = \frac{[ {a}_{0} ( T - 62 ) + {a}_{1} ] - {a}_{2}}{1 + {( \frac{t}{{a}_{3} {e}^{- a [ T - 62 ]}} )}^{( {a}_{4} ( T - 62 ) + {a}_{5} )}} + {a}_{2} \qquad ( 1 )$$

Equation (1) may, for instance, be utilized to carry out a parametric analysis relating treatment time and temperature with respect to preordained percentages of shrinkage. For example, where shrinkage cannot be observed by the clinician then a time interval of therapy may be computed on a preliminary basis. For further discourse with respect to collagen matrix shrinkage, temperature and treatment time, reference is made to the following publication:
20. Wall, et al., “Thermal Modification of Collagen” Journal of Shoulder and Elbow Surgery, 8:339-344 (1999).

With the present treatment approach, dermis is heated by radiofrequency current passing between bipolar arranged electrodes located at the interface between dermis and the next subcutaneous tissue or fat layer. To protect that subcutaneous layer, the electrodes are supported upon a polymeric thermal barrier. That barrier support is formed of a polymeric resin such as polyetherimide available under the trade designation “Ultem” from the plastics division of General Electric Company of Pittsfield, Mass. Testing of this approach is carried out ex vivo utilizing untreated pigskin harvested about 6-8 hours prior to experimentation. Such skin is, for instance, available from a facility of the Bob Evans organization in Xenia, Ohio. To position the implant at the interface between dermis and fat layer, a blunt dissecting instrument is employed to form a heating channel, whereupon an implant or wand is inserted over the instrument within that channel with its electrode or electrodes located for contact with dermis while the polymeric thermal barrier functions to protect the fatty layer. It may be noted that such polymeric material is both thermally and electrically insulative. Following implant positioning, the instrument is removed.

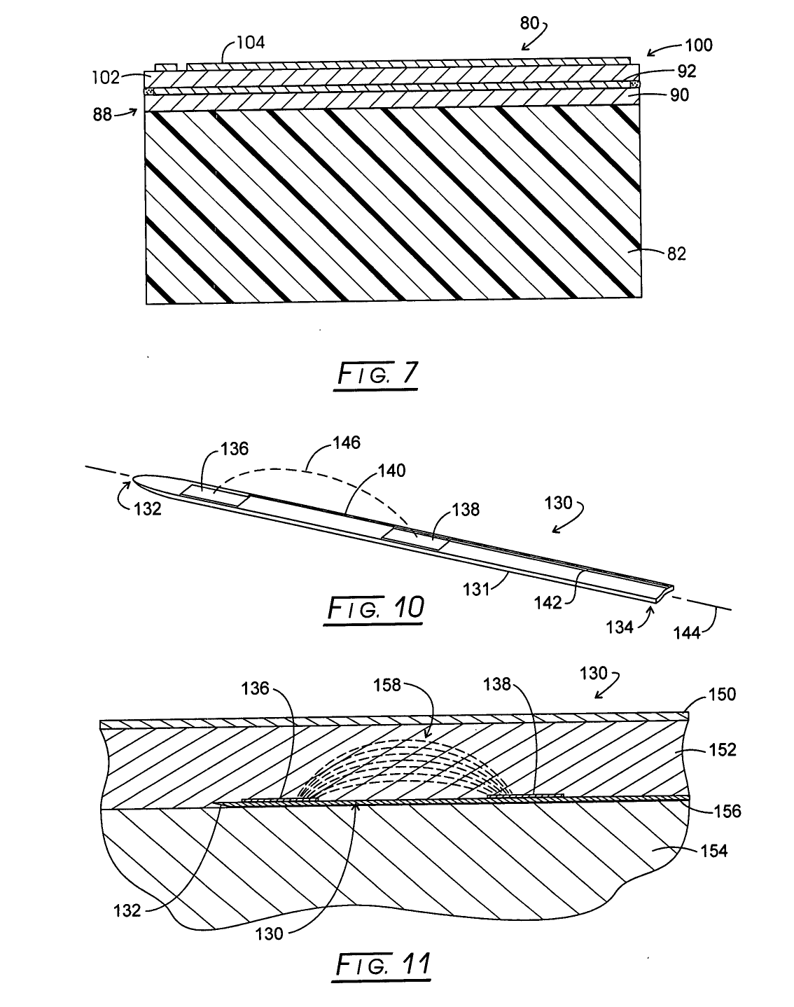

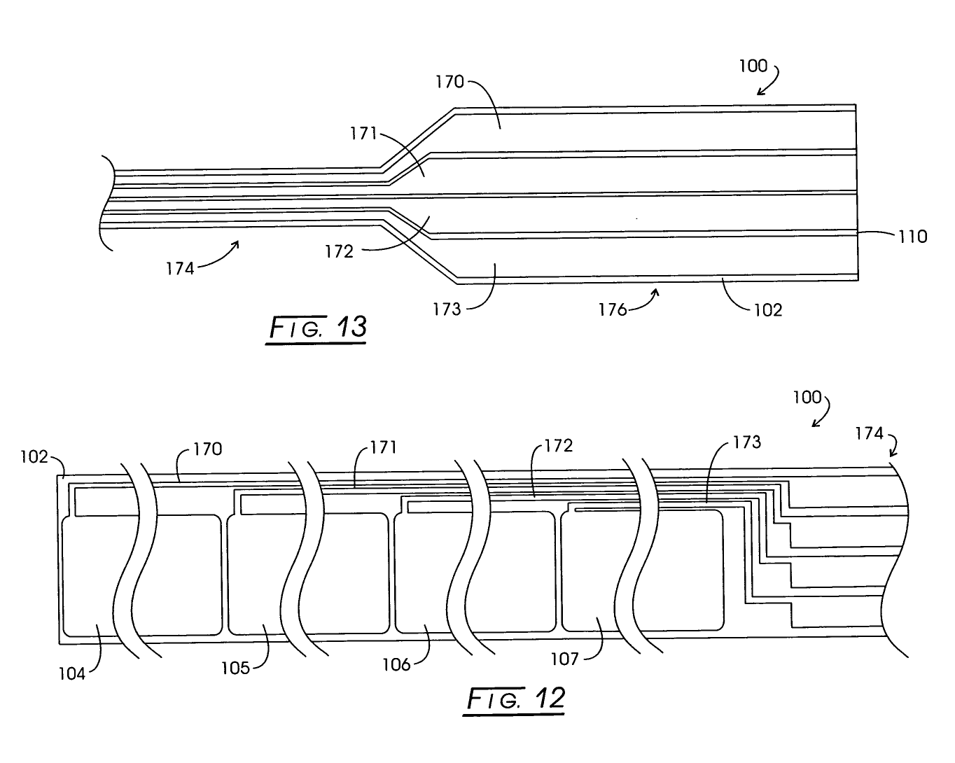

Looking to FIGS. 4 and 5, an experimental implant or wand is represented generally at 50. Implant 50 is configured with a polymeric electrically and thermally insulative support and barrier shown generally at 52. Barrier 52 is formed of the earlier described “Ultem” and is seen to extend from a tapered leading end represented generally at 54 to a trailing end represented generally at 56. This barrier exhibits a nominal thickness of 0.040 inch with a width of 0.150 inch. Adhesively secured to the upper surface 58 is a circuit assemblage formed of a thin polyimide substrate 60. Substrate 60 is generally referred to as “Kapton” and will exhibit a thickness of 0.001 inch. The upper surface 62 of the Kapton substrate affords a single electrode implemented printed circuit. The electrode of that printed circuit is identified at 64 and FIG. 4 reveals the integrally formed lead extending thereto at 66. Electrode 64 as well as its integrally formed lead 66 is formed of a gold/nickel “flash”/copper assemblage. In this regard, the copper component will exhibit a thickness of between about 0.0027 inch to about 0.0054 inch. The nickel “flash” component will exhibit a thickness of about 50 micro inches and the gold coating will exhibit a thickness of between about 8 to 12 micro inches. In general, the electrode component 64 will exhibit a length of 15 mm. Kapton layer or substrate 60 is adhesively secured to the upper surface 58 of barrier 52 and located between that barrier and the Kapton layer is a thermocouple 68 having paired leads as shown generally at 70 extending across the trailing end 56.

The principal implant or wand of the instant system is one formed with an electrically and thermally insulative barrier support which carries four electrodes intended for bipolar energization. These four electrodes exhibit a constant geometry from wand to wand, however, the length and spacing between the electrodes can be varied. Temperature at each electrode is periodically sampled by determining the resistance value of a serpentine-like resistor mounted below the electrode.

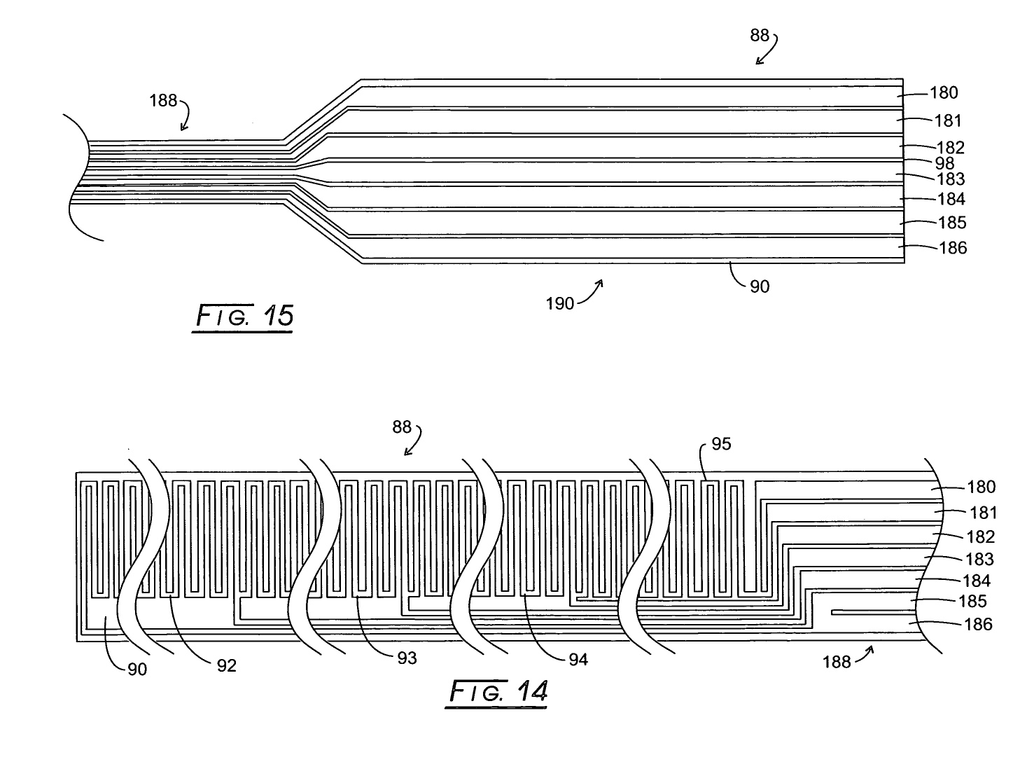

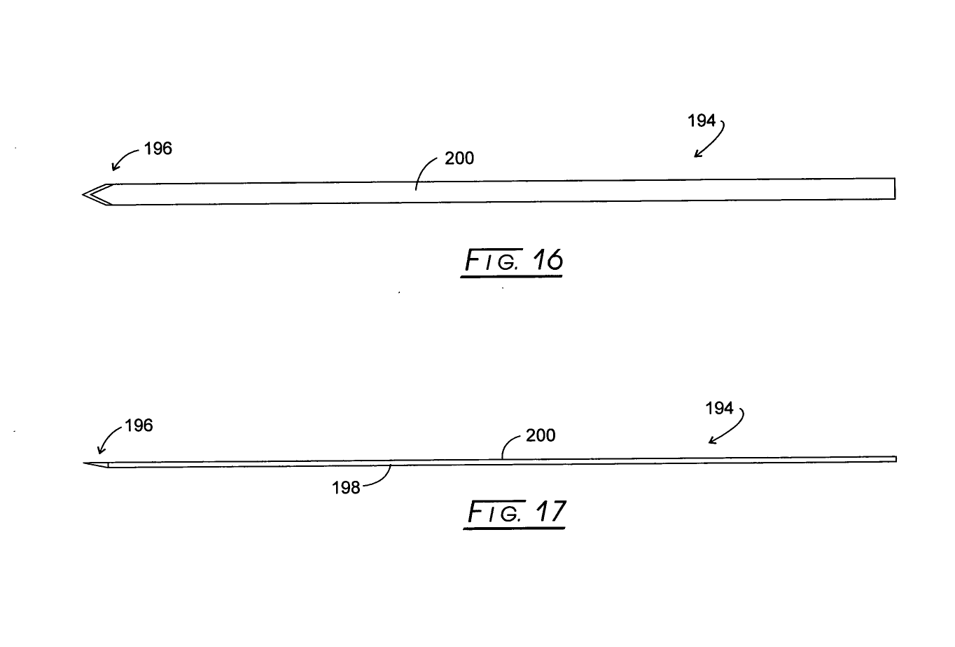

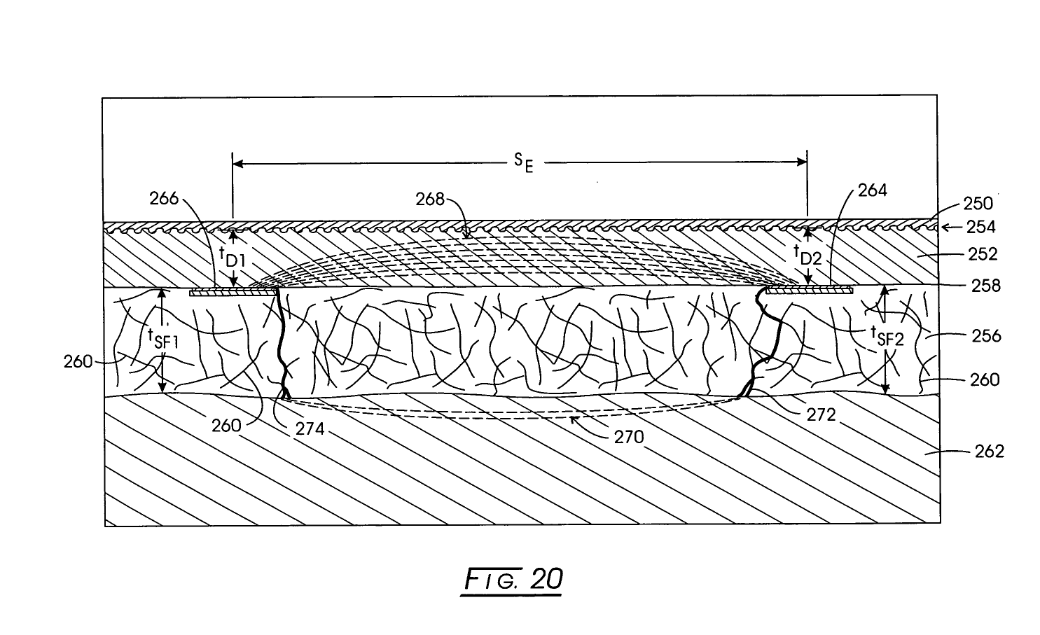

FIGS. 6-9 illustrates this principal implant or wand. Looking to FIG. 6, implant 80 is seen to be configured with a support and thermal barrier 82 formed of the earlier-described polyetheramide. Thermal barrier 82 extends from the leading end represented generally at 84 to a trailing end represented generally at 86. Note that the leading end 84 is configured somewhat as a “sled” to facilitate insertion of the implant along the surface of an introducer instrument within a heating channel. The thickness of component 82 is 0.040 inch. A flexible, resistor-based temperature sensing circuit represented generally at 88 is adhesively secured to the upward face of thermal barrier and support 82. As seen additionally in FIG. 7, circuit 88 is configured with a thin (0.001 inch) polyimide (Kapton) or flexible substrate 90 which, in turn, carries four serpentine temperature sensing resistor segments 92-95. Four-point configured leads extend from the resistor segment array extend rearwardly to an end or terminus 98. Note that the lead supporting portion of circuit 88 leading to end 98 extends over trailing end 86 of support 82. Resistor segments 92-95 and their related lead structuring are formed of one fourth ounce copper having a thickness of 0.00035 inch. Segments 92-95 are configured with trace widths of 0.003 inch and spacing between trace lengths of that same width. This permits development of a 10-15 ohm resistance measurement. Such copper thickness also permits the bending of the rearward portion of the lead structure over trailing end 86 of support 82 as represented in FIG. 9. Attachment of the flexible circuit 88 to the support 82, preferably is provided with a medical grade pressure sensitive adhesive.

Adhesively secured over the top of temperature sensing circuit 88 is an electrode supporting flexible circuit represented generally at 100. Circuit 100 is configured with a thin polyimide (Kapton) substrate or support 102 having a thickness of 0.001 inch which, in turn, supports four electrodes 104-107 along with an associated four leads extending to an end or terminus 110. As seen in FIG. 9, end 110 resides in adjacency with trailing end 86 of barrier and support 82. Electrodes 104-107 as well as their associated leads are configured with a gold/nickel “flash”/copper material wherein the copper, for example, may have a thickness of 0.0027 inch to 0.0054 inches. Correspondingly, the nickel coating may have a thickness of 50 micro inch and the gold will have a thickness of between about 8 and 12 micro inches. Circuit 100 is supported over the top of circuit 88 and is attached thereto using a medical grade pressure sensitive adhesive. By so positioning the circuit 100, the copper resistor segments 92-95 and their associated lead assemblage are sealed. Positioning of the wands may be aided by positioning indicia as represented generally at 112. In this regard, the indicia may be visually related to the entrance incision location. Indicia 112 are somewhat similar to the distance marking indicia on catheters.

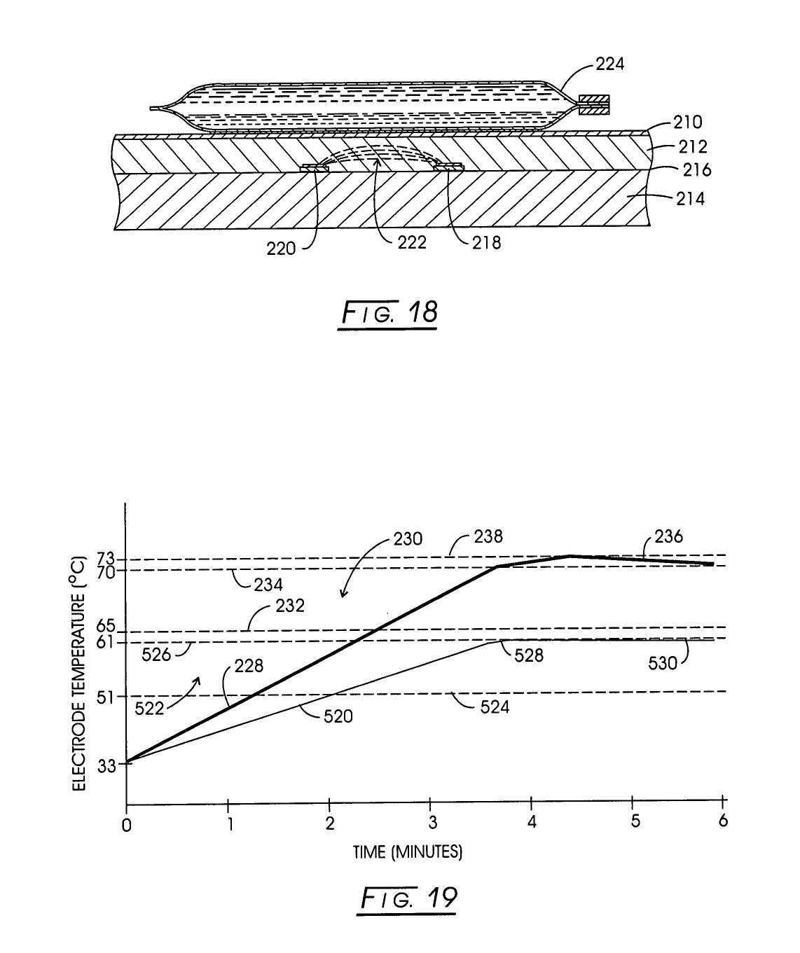

Implant 80 is designed to perform in conjunction with commercially available or “off the shelf” cable connectors. One such connector is a type MECI-108-02-F-D-RAI-SL, marketed by SAMTEC, Inc. of New Albany, Ind. With that connector, over and under contacts are provided which are in mutual alignment. Looking to FIG. 8, implant 80 is shown assembled with a polymeric connector guide identified generally at 116 having an upper slot shown generally at 118 and a lower slot represented generally at 120.

Slots 118 and 120 provide access for the contacts of a cable connector. Referring to FIG. 9, implant 80 is shown in engagement with the above-identified polymeric cable connector represented generally at 122. Note that the rearward portion of component 88 has been wrapped around end 86 of support 82. Thus, leads are available to cantilever connector contacts, two of which are shown at 124 and 126.

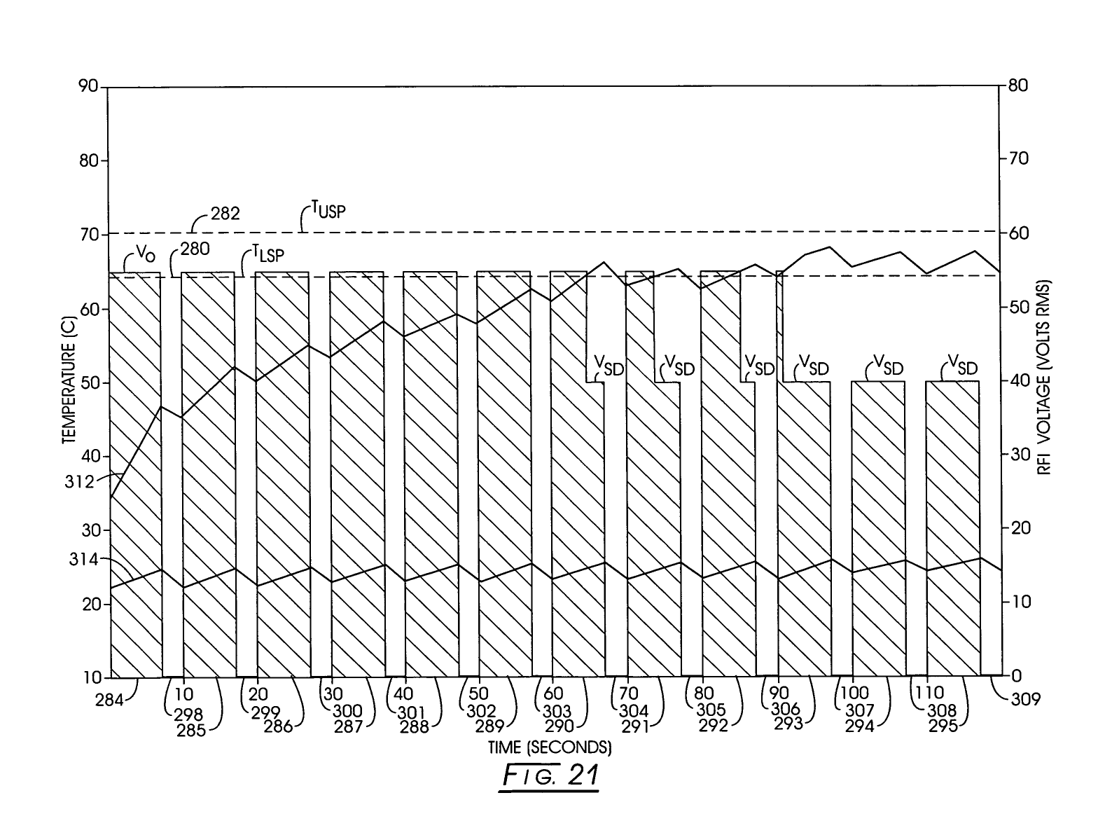

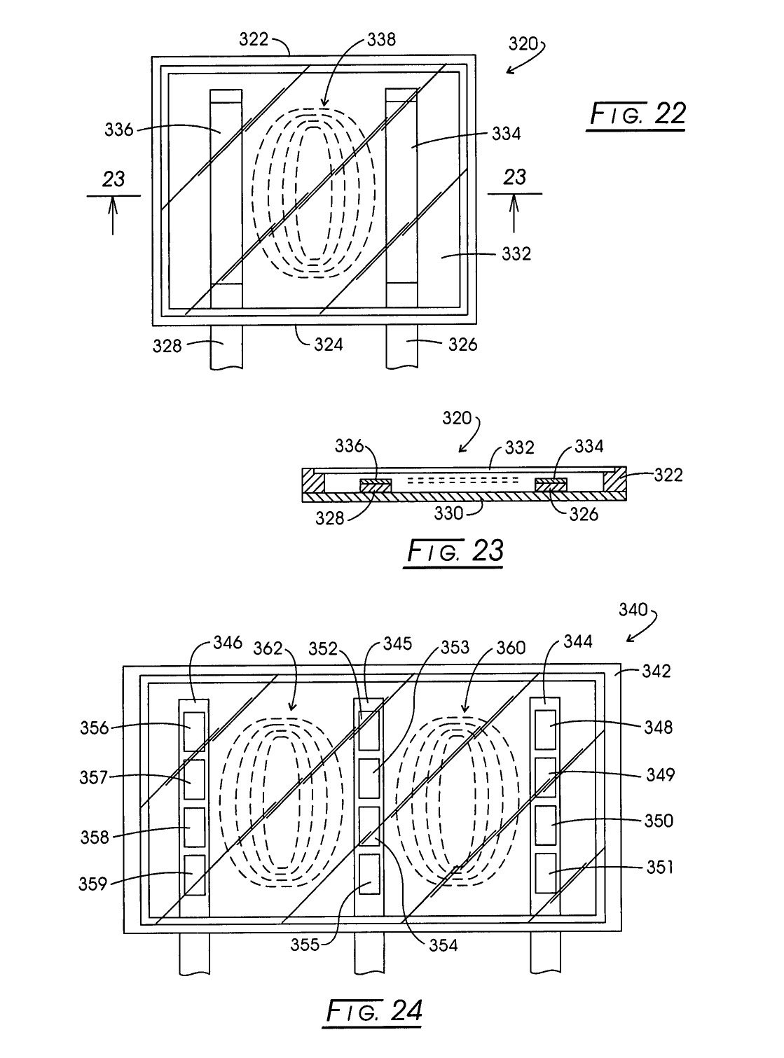

For some applications of the instant technology, only a minor amount of skin region may be involved. Under such conditions, the clinician may wish to perform with a single implant carrying spaced-apart bipolor electrodes. Referring to FIG. 10, such an implant is represented in general at 130. With the exception of the size and spacing of the electrodes, implant 130 is configured with dimensions and materials as described in conjunction with implant 80. In this regard, implant 130 is formed with a polyetheramide support and thermal barrier 131 extending from a forward end represented generally at 132 to a trailing end represented generally at 134. A flexible circuit (e.g., on a Kapton substrate) configured in the manner of component 88 and carrying two copper temperature sensing resistor segments is mounted with a pressure sensitive medical grade adhesive over the thermal barrier. The rearwardly disposed lead supporting portion (not shown) wraps over the trailing end 134 of support 131 in the manner shown in FIG. 9 in conjunction with component 88. Next, a flexible circuit component formed with Kapton carrying two spaced-apart electrodes and configured in the manner of component 100 shown in FIG. 6 is mounted over the resistor segment carrying flexible circuit in the manner described at 100 in FIG. 6. Not shown in the figure is a connector guide as described earlier at 116. The outer surface of this flexible circuit is seen to support two spaced-apart electrodes 136 and 138. Two corresponding leads as at 140 and 142 extend to the trailing end 134. Gold/nickel “flash”/copper electrodes preferably will have a length along longitudinal axis 144 of about one half inch and will be spaced apart about one inch. The bipolar association between electrodes 136 and 138 is represented by dashed curve 146. Looking to FIG. 11, schematically represented are epidermis 150; dermis 152; and next adjacent subcutaneous tissue or fat layer 154. Implant 130 is located within a heating channel at the interface 156 between dermis 152 and next adjacent subcutaneous tissue layer 154. When electrodes 136 and 138 are excited in bipolar fashion with radiofrequency energy, a current flux path represented generally at 158 will function to heat a small zone of dermis 152.

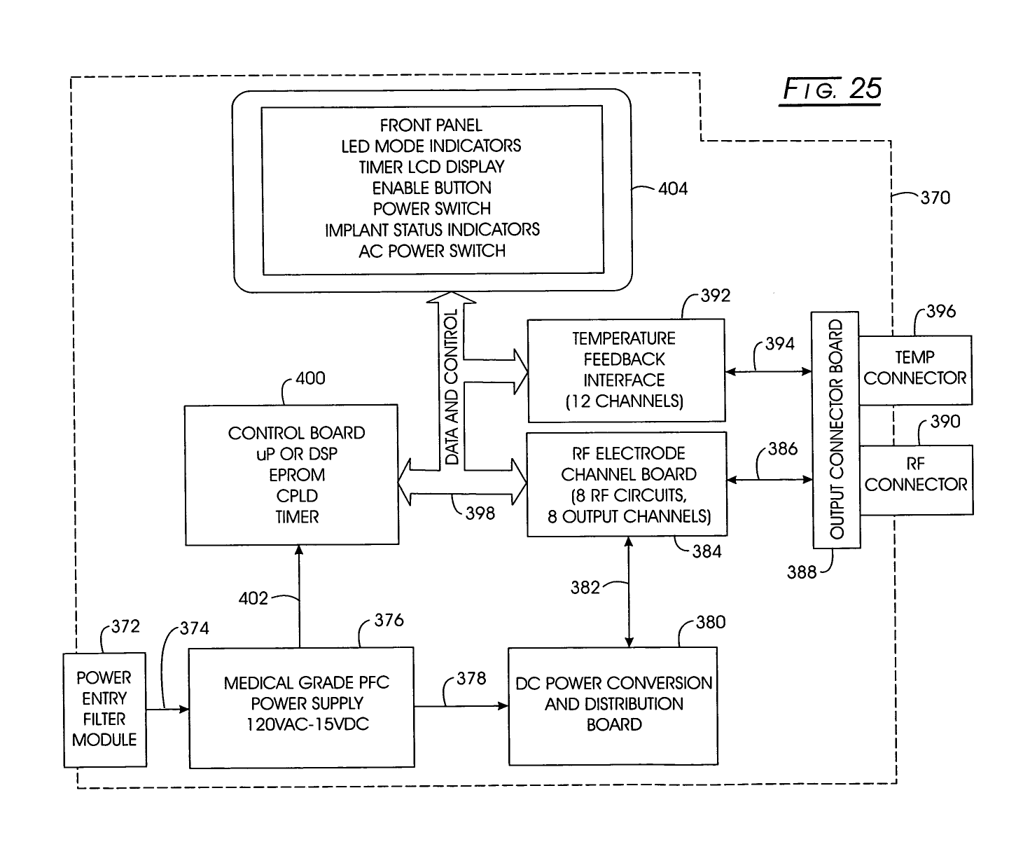

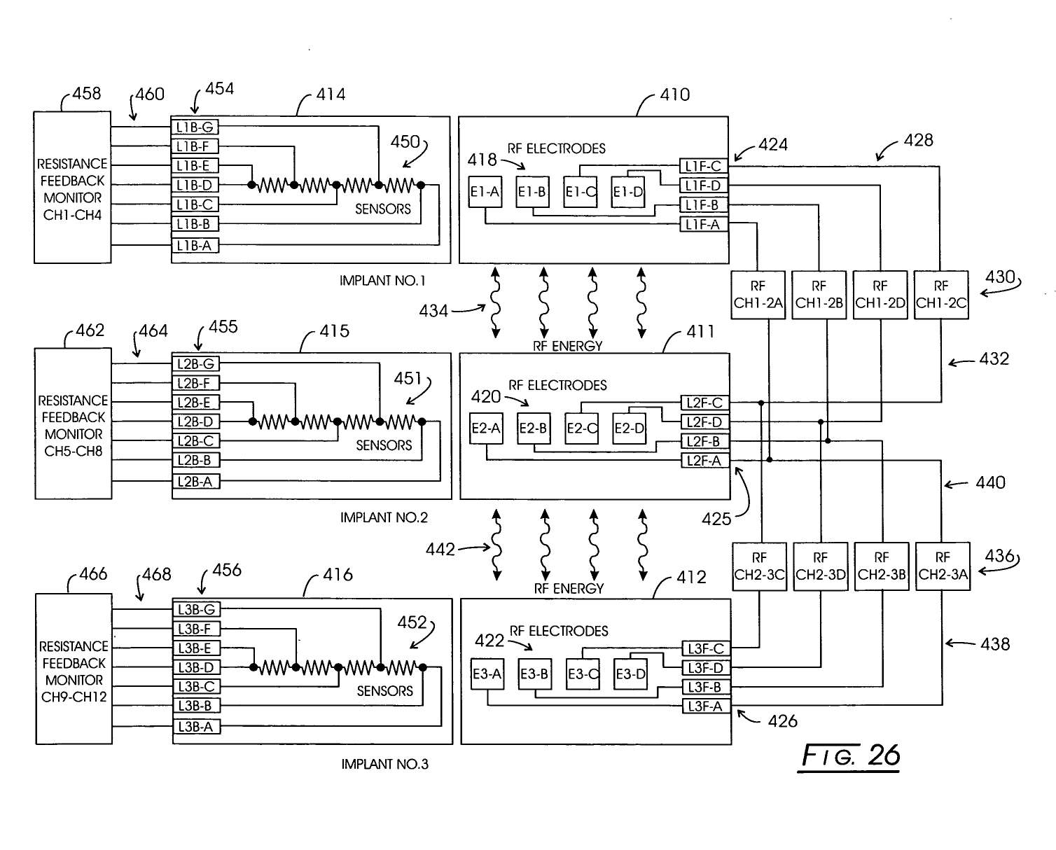

Referring to FIGS. 12 and 13, flexible circuit component 100 as described in connection with FIGS. 6-9 is illustrated at an enhanced level of detail. In FIG. 12, the four electrodes 104-107 reappear as supported upon polyimide substrate 102. Leads 170-173 extend from integral connection with respective electrodes 104-107 whereupon they are expanded in width within an intermediate region of component 100 as represented in general at 174. The widths are still further expanded at a rearward region represented generally at 176. It may be recalled that electrodes 104-107 and their associated lead traces 170-173 are formed of gold plated/nickel “flash”/copper material. The lead traces 170-173 are electrically insulated with a coverlay where contactable with tissue.

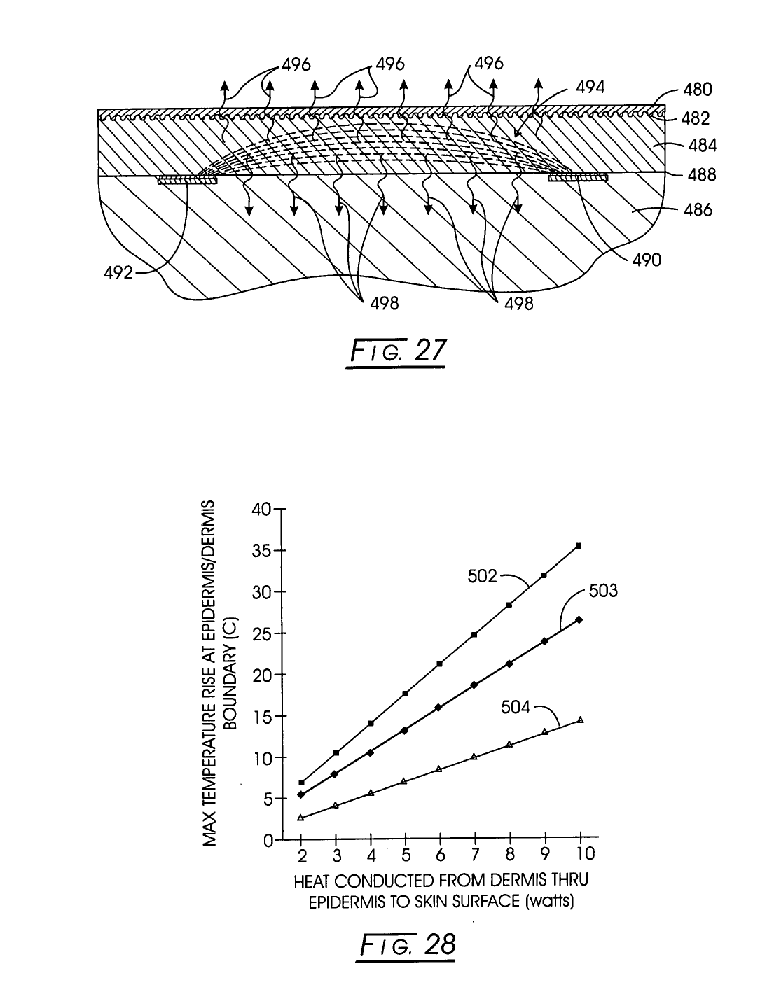

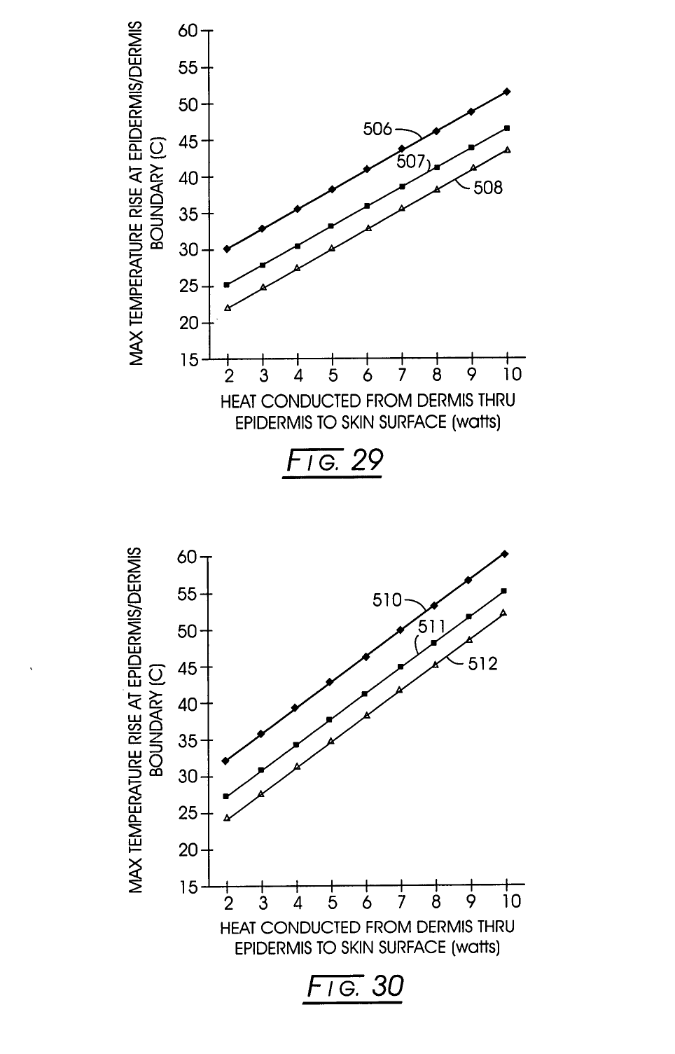

Referring to FIGS. 14 and 15, an enlarged broken away view of temperature sensing circuit 88 is presented. FIG. 14 reveals the flexible circuit substrate 90 (Kapton) supporting four copper resistor segments 92-95. Segments 92-95 are aligned with corresponding respective electrodes 104-107 such that they are in thermal transfer relationship therewith to evaluate the temperature of the electrodes. These four sensing resistor segments are addressed by lead traces 180 and 186 which are arranged to provide a four-point interconnection. In this regard, lead traces 180-186 provide a low level d.c. source current, while leads 181-185 serve to provide a temperature sensor output. Note that the widths of leads 180-186 are expanded in an intermediate region represented generally at 188. Looking to FIG. 15, that intermediate region 188 reappears at a lesser level of magnification, whereupon the leads are again expanded in width at a rearward region represented in general at 190. Inasmuch as component 88 is embedded under component 100 and attached thereto by pressure sensitive medical grade adhesive, no additional electrically insulative features are called for.

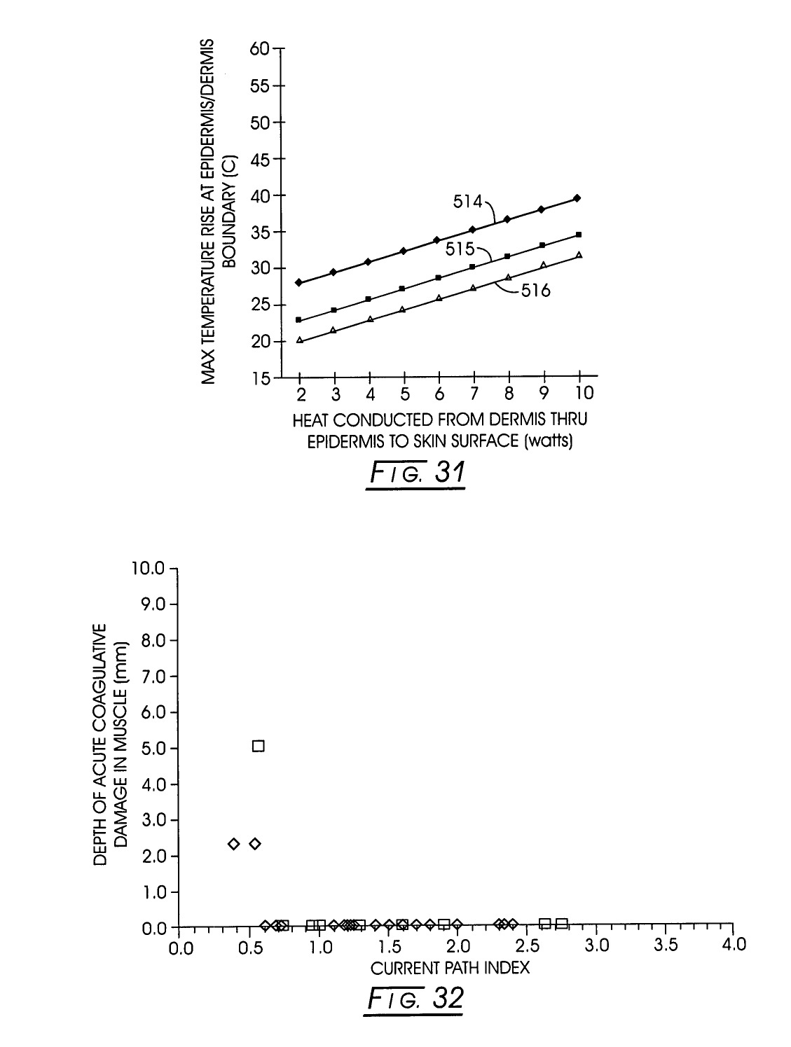

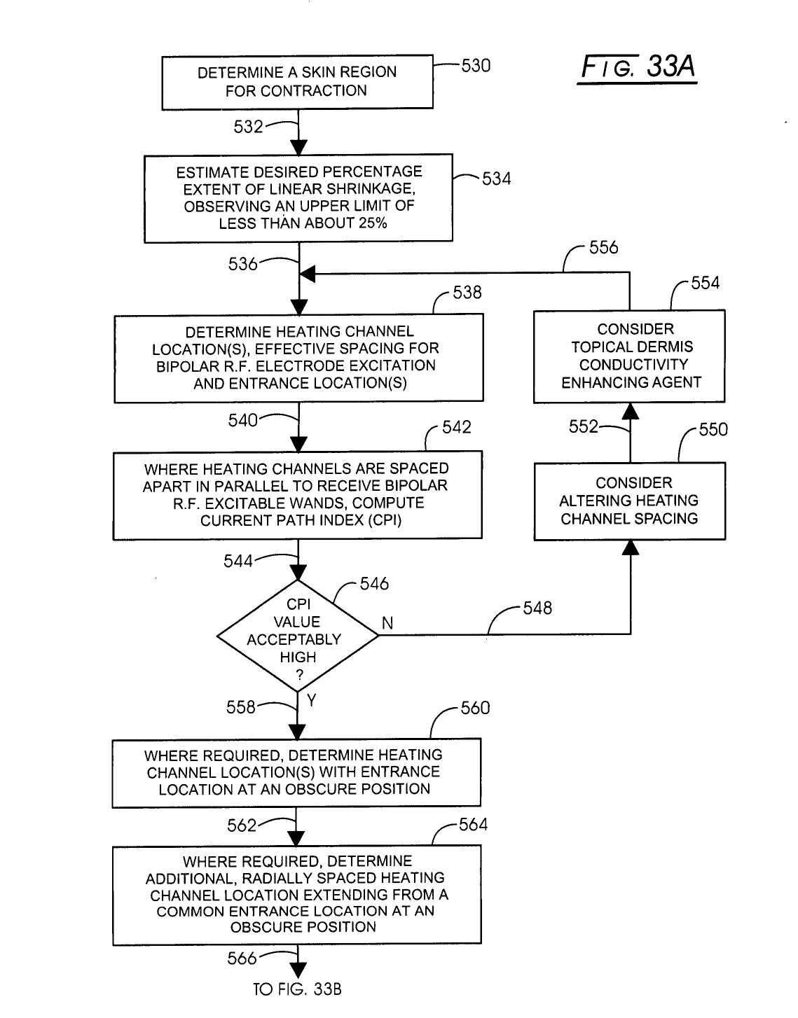

The positioning of implants or wands as at 50, 80 and 130 at the interface between dermis and the next subcutaneous tissue layer, involves the preliminary formation of a heating channel utilizing a flat needle introducer or blunt dissector. Looking to FIG. 16, such an introducer is represented generally at 194. Device 194 is, for instance, 4 mm wide and is formed of a stainless steel, for example, type 304 having a thickness of about 0.020 inch to about 0.060 inch. Its tip, represented generally at 196, is not “surgically sharp” in consequence of the nature of the noted interface between dermis and fat layer. However, looking to FIG. 17, it may be observed that the tip 196 slants upwardly from its bottom surface 198 to evoke a slight mechanical bias toward dermis when the instrument is utilized for the formation of a heating channel. In utilizing an introducer as at 194, the introducer is employed to form a heating channel from a scalpel formed entrance incision. Following placement and formation of the heating channel, a wand or implant is slid over the top surface 200 of the introducer. Upon positioning the implant or wand, then the introducer 194 is removed leaving the implant or wand in place.

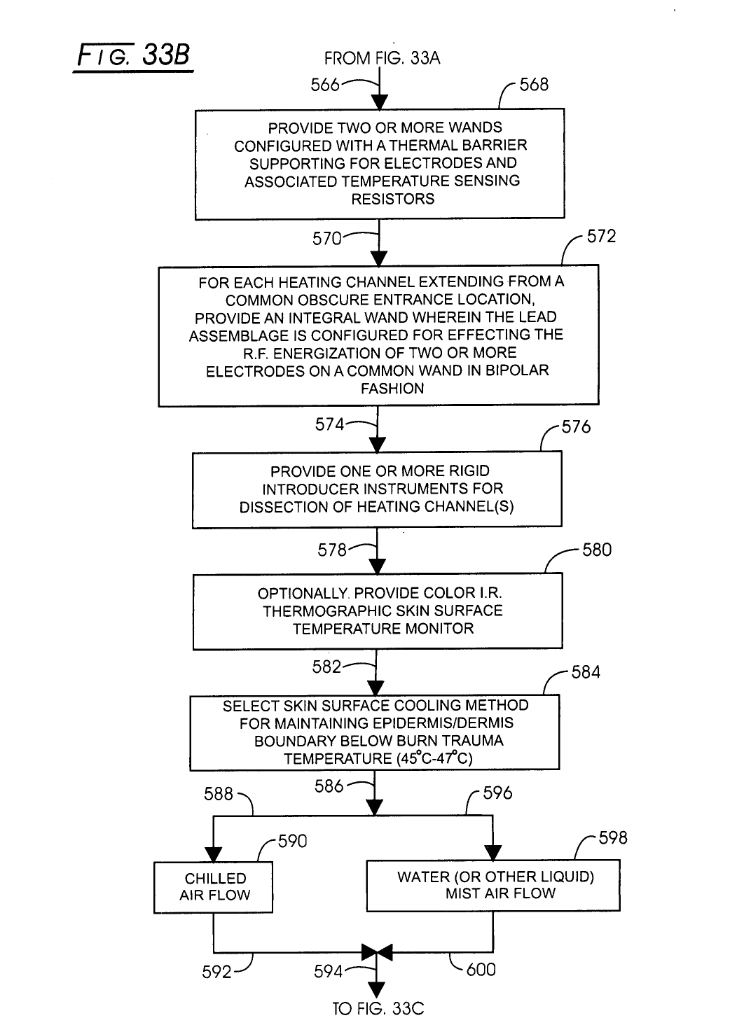

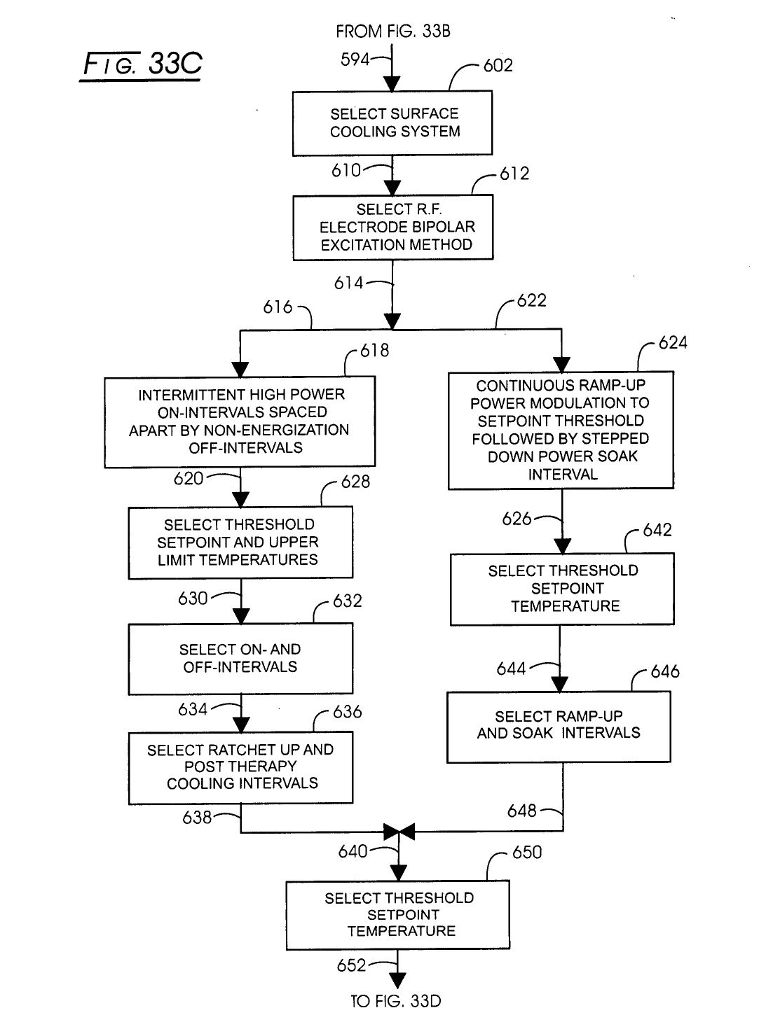

Looking to FIG. 18, a schematic representation of earlier animal (pig) studies is set forth. The studies were both ex vivo and in vivo. In the figure, epidermis is depicted at 210; dermis at 212 and the next subcutaneous tissue or fat layer at 214. The interface between the fat layer 214 and dermis 212 is identified at 216. At this interface, bipolar implants as 218 and 220 were positioned. These implants are configured as shown at 50 in FIGS. 4 and 5. R.F. current flux is represented as extending between the electrodes of the wands 218 and 220 by a grouping of dashed lines represented generally at 222. Cooling for this early approach was carried out by a skin temperature control unit implemented as a water filled flexible polymeric bag container 224. Device 224 functions as a constant temperature heat sink. R.F. implemented power was applied between the bipolar electrodes of implants 218 and 220 on what may be referred to as a continuous mode. In this regard, looking to FIG. 19, a plot of desired electrode temperature with respect to therapy time in minutes is presented wherein a controlled ramping-up of electrode temperature into a collagen shrinkage domain over a ramp interval is followed by what is referred to as a “thermal soak” interval. In the figure, the ramp-up region of an electrode temperature-time curve is shown at 228. Between about 65° C. and 70° C. there is established a collagen shrinkage domain represented generally at 230. Shrinkage domain 230 is seen to extend between the dashed line level 232 corresponding with the collagen shrinkage threshold temperature of 65° C. and dashed level line 234 corresponding with a transition temperature of 70° C. Curve portion 228 is seen to transition at that temperature level which occurs at about 4 minutes elapsed therapy time. Next, as represented at soak interval curve portion 236, during a soak interval of about 2 minutes, electrode temperature may be slightly elevated, for example to a maximum level of 73° C. as represented at dashed line 238. In general, a reduced power input may be applied during the soak interval represented at curve portion 236.

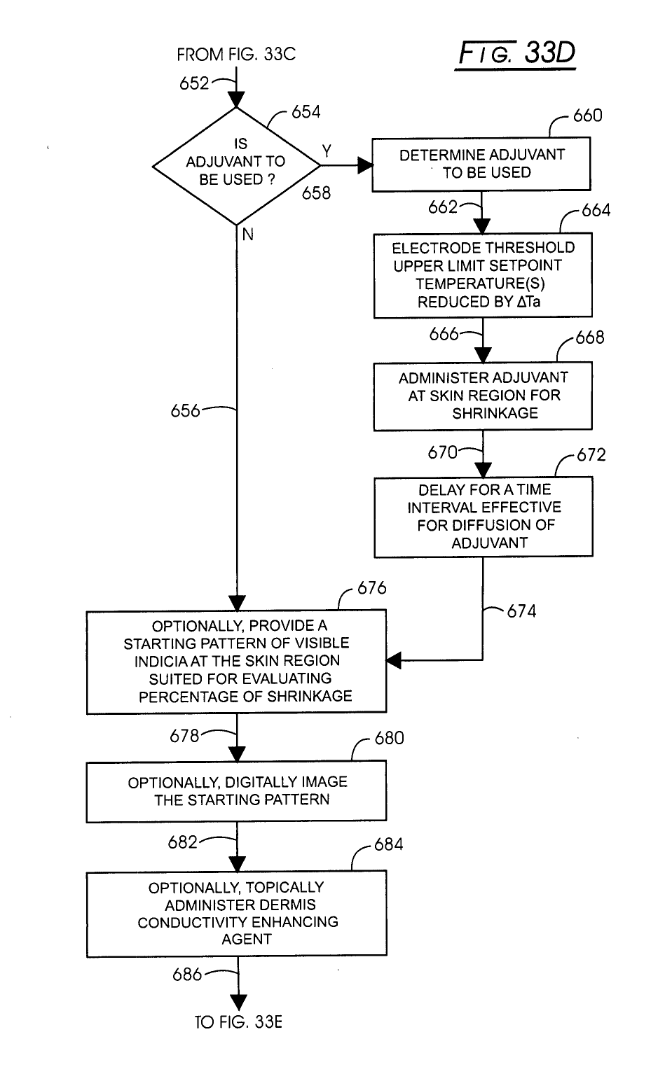

With the arrangement depicted in FIGS. 18 and 19, it was found that even in the same animal experiment burns at the epidermis were, on occasion witnessed. In general, if the dermis was found to be thin, a large temperature gradient would be developed across the interface between dermis 212 and epidermis 210. In consequence, the utilization of water-filled flexible heat sinks as at 224 was discontinued. (The weight of the water-filled container 224 may have compressed the dermis as well as restricted skin shrinkage.) Pathology findings further revealed evidence of occasional current tracking or shunting into and through the muscle layer during R.F. intradermal heating. On occasions in which this occurred, the R.F. current level flowing through the muscle was sufficient to cause acute coagulative damage within the muscle layer. In this regard, a more elaborate schematic representation of skin, subcutaneous fat and muscle is presented in FIG. 20. In the figure, epidermis is represented at 250 as discussed in connection with FIG. 3. Epidermis 250 exhibits downwardly depending rete ridges which may be considered in conjunction with a determination of its thickness. The epidermis overlies dermis 252 at an interface represented generally at 254. Below dermis 252 is a subcutaneous fat layer 256 and the interface between fat layer 256 and dermis 252 is shown at 258. Within the fat layer 256 fibrous septae are represented, certain of which are identified at 260. Fat layer 256 overlays muscle as represented at 262.

Two wands or implants which may be configured as at 50 in FIGS. 4 and 5 or as at 80 as shown in FIGS. 6-9 are represented at 264 and 266 located at the interface 258 between dermis 252 and fat layer 256. The bipolar path of current within dermis 252 is represented generally at 268 extending between the electrodes of implants 264 and 266. A current flow path additionally is shown in general at 270 within muscle 262. Current path 270 may possibly be a result of conduction through conducting fibrous septae as represented schematically at 272 and 274.

Literature studies were carried out with respect to the electrical resistivity at 37° C. of dermis 252, subcutaneous fat layer 256 and muscle as at 262. The results of those studies are tabulated in Table 1 below. In the table, data represented at lines 1-4 were derived from Chenng, K. et al. Bioelectromagnetics, 17:458-466 (1996). Data at lines 5 and 8 were derived from Polk, C. and Postow, E., CRC Handbook of Biological Effects of Electromagnetic Fields (1988). Data at line 5 additionally was derived from Hemingway, A., et al., Am. J. Physiol., 102:56-59 (1932). Data at line 6 was derived Duck, F. A., Physical Properties of Tissue, Academic Press (1990), Table 6.13. Data at line 7 was derived from Stoy, R. D., et al., Dielectric Properties of Mammalian Tissue (1982), page 505. Data at lines 9 and 10 was derived from Schwann, H., Physical Techniques in Biological Res., Oster, G. (ed), pp 332-333 (1963).

TABLE 1
Electrical Resistivity of Dermis, Subcutaneous Fat and Muscle

| | | | | | Electrical Resistivity at |
|---|---|---|---|---|---|
| | Type of | | | Frequency | 37 C. |
| Line | Tissue | Species | Direction of Correct Flow | (kHz) | (ohm-cm) |
| 1 | Dermis | Porcine | Parallel to skin surface | Rectangular pulsed current | 263 |
| 2 | | Porcine | Perpendicular to skin surface | Rectangular pulsed current | 370 |
| 3 | Subcutaneous Fat | Porcine | Parallel to skin surface | Rectangular pulsed current | 1,350 |
| 4 | | Porcine | Perpendicular to skin surface | Rectangular pulsed current | 2,220 |
| 5 | | Human | Nonoriented | 100 | 2,500 |
| 6 | Muscle | Porcine | Nonoriented | 1,000 | 172 |
| 7 | | Human | Nonoriented | 1,000 | 163 to 200 |
| 8 | | Rat(skeletal) | Nonoriented | 1,000 | 119 |
| 9 | | Human | Nonoriented | 100 | 170 to 210 |
| 10 | | Human | Nonoriented | 1,000 | 160 to 210 |

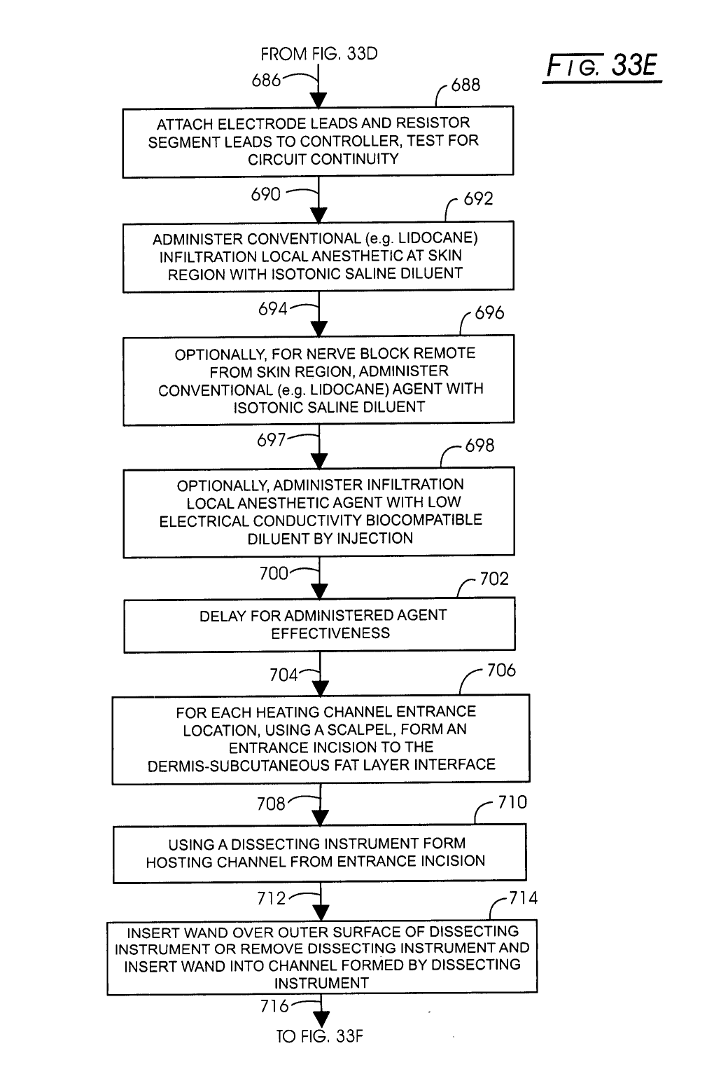

A determination was made to replace the heat sinks illustrated at 224 in FIG. 18 with a cooling airflow, for example, a chilled airflow or a mist airflow. In addition, an aluminum heat sink was also contemplated. While such cooling will be effective in a continuous mode of electrode energization as discussed in connection with FIG. 19 based upon a later described computational evaluation referred to as a current path index (CPI), an intermittent mode of bipolar electrode energization was developed wherein a select higher power level is employed for a sequence of energization on-intervals time spaced apart by non-energization off-intervals. Such an electrode powering algorithm is diagramed in FIG. 21. In that figure, time in seconds is represented along an abscissa, while electrode temperature in degrees centigrade is represented along a left ordinate and R.F. volts (RMS) is represented along a right hand ordinate. The lower threshold setpoint, for example, representing a temperature, T_{LSP}, of, for example, 65° C. is represented at dashed line 280, while an upper limit electrode temperature setpoint (Tᵤₛₚ) of, for example, 71° C. is represented at dashed line 282. Power application or energization on-intervals, for example, of 7 second duration, are represented at 284-295. These intervals are interspersed or separated by power-off intervals 298-309 which, for example, may have duration of 3 seconds. In general, the power on-intervals will range from about 1.0 seconds to about 8.0 seconds, while the power-off intervals will range from about 1.0 seconds to about 3.0 seconds. As represented by the voltage level V₀ associated with power-on intervals 284-293 during an initial ratchet-up interval essentially maximum constant power is applied across the electrodes. This is represented at ratcheting curve 312. Such maximum power is applied as long as curve 312 falls below the lower temperature threshold setpoint represented at dashed line 280. Note, additionally, that during the off-intervals 298-309, the temperature of the electrodes drops slightly. The rationale for the intermittent mode approach is based upon the dual requirements of (1) apply heating for a sufficiently long period (i.e., the power-on time or interval) so as to raise the average temperature of the dermis layer during those successive on-interval cycles until the setpoint temperature represented at line 280 is reached; (2) interrupting the heating for a sufficient off-interval to effect adequate cool-down of the epidermis to limit its maximum temperature rise during the complete intra-dermal heating period comprised of multiple off-cycles; and (3) interrupting the heating for periods sufficiently short to avoid over-cooling the dermis layer sought to be heated to about 63° C. and above. Skin surface temperature is represented at curve 314. Note that it remains just above 20° C. and intermittently drops during the off-intervals 298-309.

It may be observed in the figure that ratcheting up somewhat terminates when curve 312 passes through and above the lower threshold setpoint temperature represented at dashed line 280. This is shown first to occur in conjunction with power-on interval 290. As dashed line 280 is passed, a stepped-down voltage, V_{SD}, is applied. The figure reveals that with respect to power-on intervals 290-293 stepped-down voltage is present for a portion of such interval. However, with respect to power-on intervals 294 and 295, the stepped-down voltage V_{SD}, is applied during the entire interval as curve 312 lies between lower threshold temperature as represented at dashed line 280 and upper limit temperature as represented at line 282. The stepped-down voltage, V_{SD}, generally will be a percentage of the full power voltage V₀, for example, 65%. For instance, if V₀, is 50 volts (RMS) then the stepped-down voltage, V_{SD}, is 32.5 volts (RMS). During the therapy session represented at FIG. 21, temperature should be monitored, for example, utilizing the resistor segments as described in conjunction with FIGS. 14 and 15. In general, the full extent of the epidermis thickness needs to be maintained at less than about 45° C. A controller should poll all of the electrode temperature sensing resistors, for example, eight resistor segments about every second. Then, based upon the temperature of each resistor segment, the controller determines whether the applied voltage to any electrode pair to be the maximum selected ratchet up-value (V₀) the step-down value (V_{SD}) or the value of zero volts. That latter value represents a system shut-down which will occur should curve 312 extend above the upper limit setpoint temperature represented at dashed line 282.

Bench tests have been carried out to evaluate the electric field performance of the implant or wand carried electrodes performing in both a continuous mode and the intermittent mode described in connection with FIG. 21. These tests were carried out with chicken egg white in view of its unique properties wherein it changes from a transparent medium to opaque white in the narrow temperature range of 60° C. to 61° C.

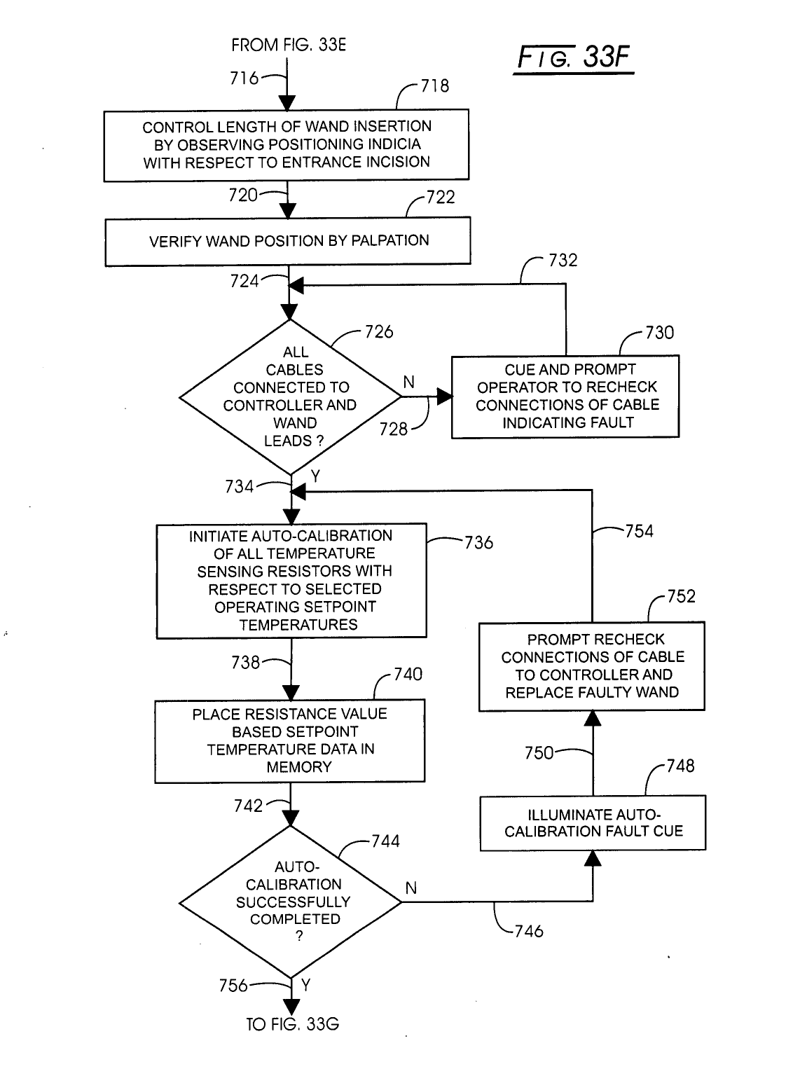

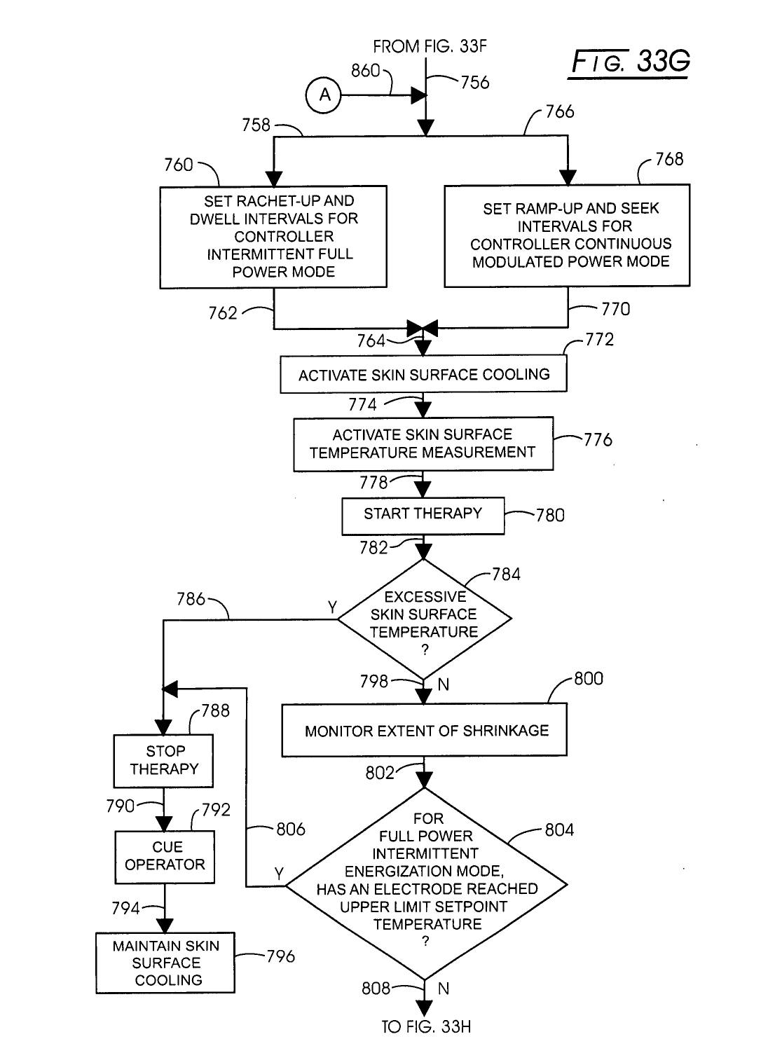

Referring to FIGS. 22 and 23, an electric field test cell is represented generally at 320. Cell 320 is intended for retaining transparent chicken egg white and is structured for the utilization of two implants or wands as described earlier at 50 in conjunction with FIGS. 4 and 5. Cell 320 is configured with an electrically insulative rectangular peripheral frame 322, the sides of which exhibit an outer dimension of 1.5 inch. Frame 322 as seen in FIG. 23, is configured with slots at frame edge 324 which receive two single electrode implants schematically represented at 326 and 328. FIG. 23 further reveals that frame 322 is adhesively coupled to a transparent glass base 330 and is covered with a 0.008 inch thick quartz glass “cover slip” 332. The electrodes carried by implants or wands 326 and 328 are identified respectively at 334 and 336. Each of these electrodes was 15 mm in length and 3 mm in width and the two were spaced center-to-center 15 mm. A digital microscope recorded the test procedure.

Upon energization in bipolar fashion of electrodes 334 and 336 the central region between the electrodes commenced to become opaque and that opacity moved toward and ultimately covered the electrodes. Such opacity is represented by oval structured dashed lines represented generally at 338. The test revealed that the electrodes were working in concert, heating up at the same rate without hot spots.

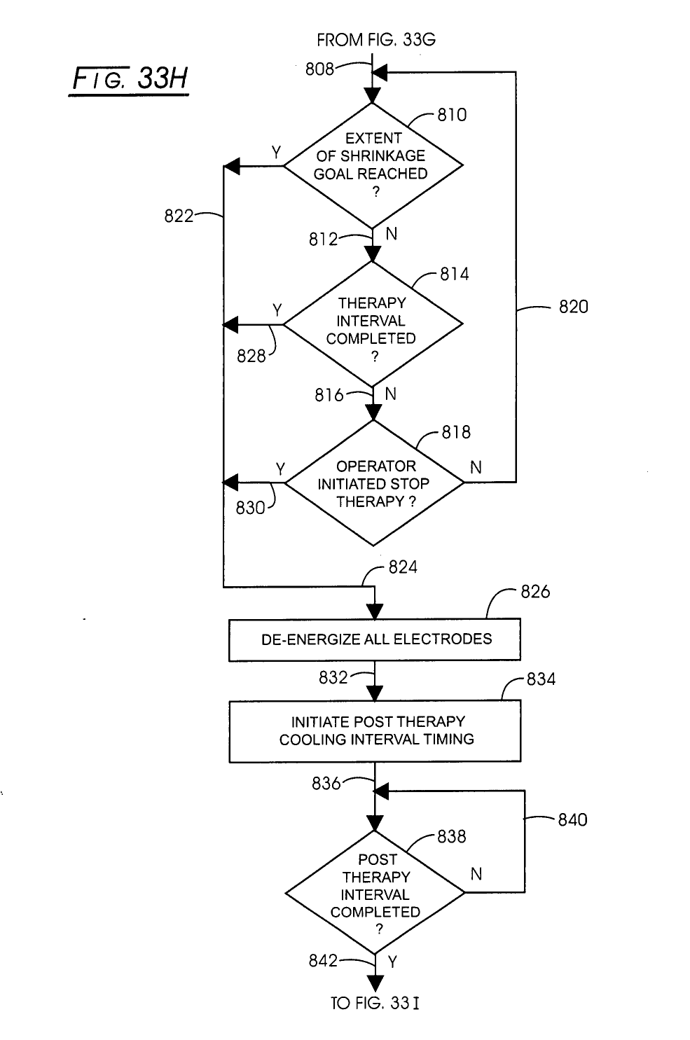

A next electric field cell test was carried out utilizing three, 4-electrode wands or implants as described through FIGS. 6-9. The test cell is schematically detected in FIG. 24 in general at 340. Test cell 340 was configured with a frame and glass bottom in the same manner as cell 320 but at a larger dimension suited for retaining three wands or implants. The rectangular frame of cell 340 is seen at 342. To permit improved air cooling, the glass cover slip was replaced with a transparent sapphire (AL₂0₃) window having a thickness of 0.030 inch. The three wands or implants employed with the cell 340 are shown at 344-346. Electrodes at wand 344 are identified respectively at 348-351. Corresponding bipolar associated electrodes at wand or implant 345 are identified at 352-355. These electrodes 352-355 are “shared” electrodes inasmuch as they also perform in bipolar fashion with respective electrodes 356-359 of wand or implant 346. R.F. bipolar energization of the electrodes, as before, created an opacity commencing at the midpoint between them as represented at dashed opacity symbols identified generally at 360 and 362. It was observed that even though the electrodes of wand or implant 345 were performing in conjunction with two outboard electrodes, no hot spots were evolved in consequence of this dual functioning.

Temperature evaluating resistor segments have been discussed inter alia, in connection with FIGS. 6-9 and 14-15. Considering the functioning of these segments, once a wand or implant has been located within a heating channel and preferably following the activation of skin surface cooling, the temperature of resistor segment s is determined. For example, this predetermined resistor segment temperature, T_{RS,t0}, based on an algorithm related to the measured skin surface temperature, T_{skin,t0,} may be expressed as follows:

T_{RS,t0}=f(T_{skin,t0}). (2)

As an example, this computed temperature may be 35° C. Also predetermined is the treatment target or the setpoint temperature. That temperature may be based upon radiofrequency heating in a continuous mode as described in connection with FIG. 19 or in an intermittent mode as discussed in connection with FIG. 21.

When the controller is instructed to commence auto-calibration the following procedure may be carried out:
a. The controller measures the resistance of each resistor segment preferably employing a low-current DC resistance measurement to prevent current induced heating of those resistors.
b. Since the resistor component is metal having a well-known, consistent and large temperature coefficient of resistance, a having a value preferably greater than 3000 ppm/° C. (a preferred value is 3800 ppm/° C.), then the target resistance for each Resistor Segment can be calculated using the relationship:

R_{RSi,target}=R_{RSi,t0}(1+α*(T_{RS,t}−Tₜ₀)) (3)
where:
R_{RSi,t0}=measured resistance of Resistor Segment, i, at imputed temperature of Resistor Segment under skin, T_{RS,t0}
α=temperature coefficient of resistance of resistor segment.
T_{RS,t}=target or setpoint treatment temperature.
T_{RS,t0}=Imputed temperature of RF electrodes residing under the skin and prior to the start of any heating of them.

For four-point sensor resistor connections, no accommodation need be made for the impedance exhibited by the cable extending to the controller. Temperature evaluations are made intermittently. For instance, for a continuous mode of performance they may be made every 500 milliseconds and a sampling interval may be quite short, for instance, two milliseconds. For intermittent mode performance, as discussed above, the interval for temperature management in voltage control may be approximately one second with respect to the measurement of temperature of all electrodes involved. Again, the sampling interval may be quite short, for example, two milliseconds.

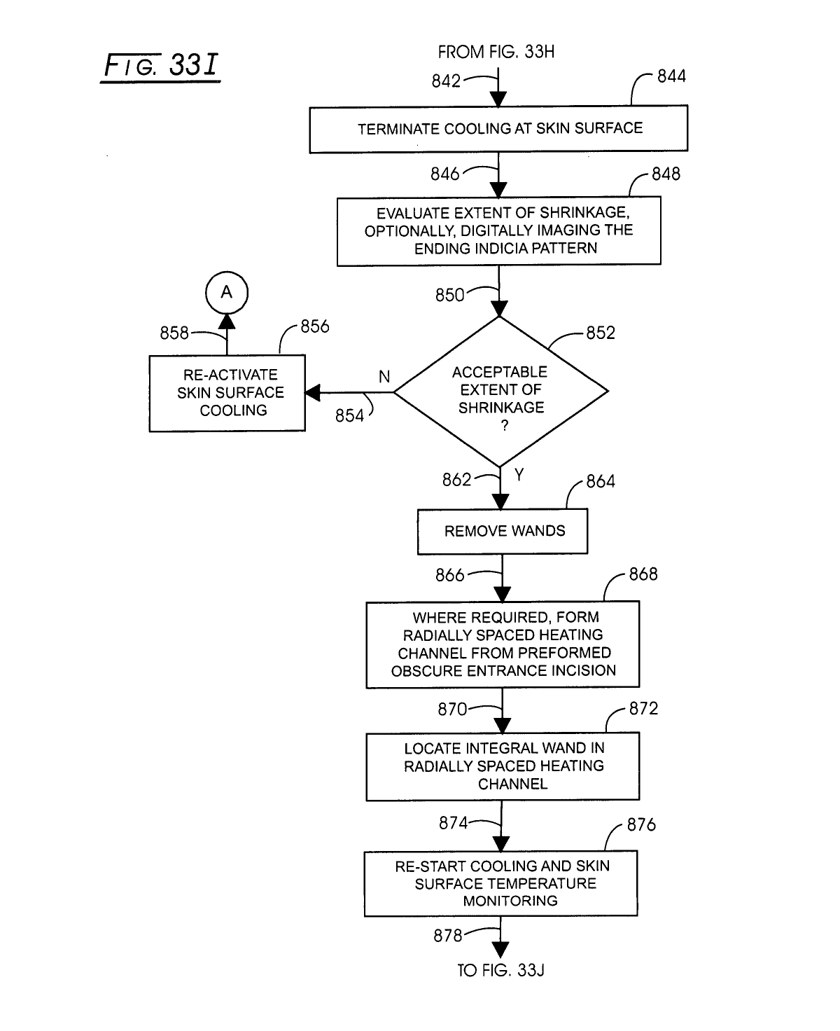

Referring to FIG. 25, a block diagram is presented within dashed boundary 370 representing a control console performing, for instance, with three implants, each supporting four R.F. electrodes and an associated four temperature sensing resistor segments. (Recall FIG. 24.) In the figure, a power entry filter module is represented at block 372 providing a filtered a.c. input as represented at arrow 374 to a medical-grade power supply with power factor correction (PFC) as represented at block 376. By providing PFC correction at this entry level to the control circuitry, the console will enjoy a somewhat universal utilization with various worldwide power systems. The d.c. output from power supply 376 is provided, as represented at arrow 378 to a d.c. power conversion and distribution board represented at block 380. As represented by dual arrow 382, logic power and radiofrequency energy inputs are provided to a radiofrequency electrode channel board represented at block 384. Channel board 384 will exhibit a topography incorporating eight bipolar radiofrequency circuits and an associated eight output channels. As represented by the interfacing dual arrow 386 and block 388, the output channels are directed to an output connector board which is operatively associated with the radiofrequency electrode connector as represented at block 390. Also associated with the output connector board 388 is the twelve channel resistor segment temperature feedback interface represented at block 392 and dual interface functioning arrow 394. The connectors associated with the function of arrow 394 are represented at block 396. Control into and from the temperature feedback interface 392 and the R.F. electrode channel board 384 is represented at control bus or arrow 398. The circuit distribution function at bus 398 is seen to be functionally associated with a control board represented at block 400. Such control may be implemented, for instance, with a microprocessor or digital signal processor and will include memory (EPROM). It may also be implemented with a programmable logic array or device (CPLD), and a timing function. Logic d.c. power supply is directed to the control function 400 as represented at arrow 402. As represented at bus 398 and symbol 404 the console 370 incorporates a front panel having user control input as well as displays. In this regard, as listed in the symbol, the console employs an a.c. power switch; implant status indicator; a power switch; an enable button or switch; a timer LCD display; a light emitting diode (LED) mode indicator. Additional inputs, for example, for intermittent mode operation may be power-on times, off-time intervals, setpoint levels, step-down voltage at setpoint temperature, and the like.

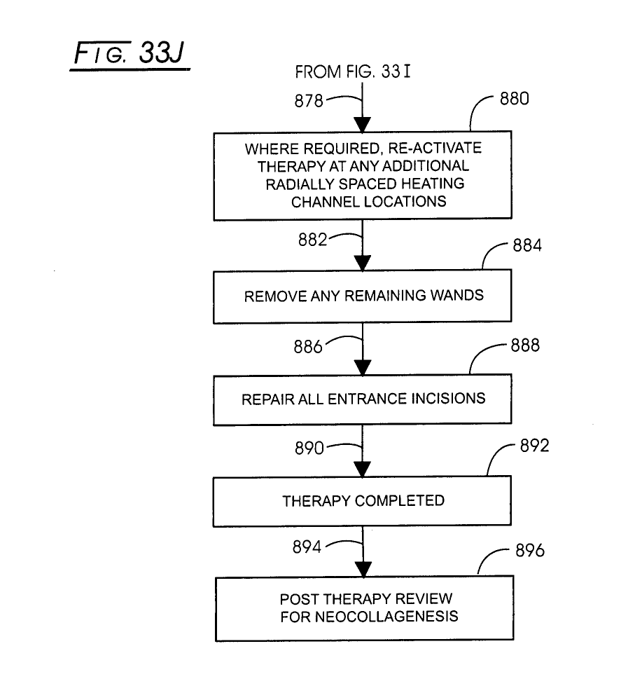

Referring to FIG. 26, schematic representation of the flexible circuit assemblies for three implants numbered 1-3 are presented in combination with the functions of resistance feedback monitoring and bipolar radiofrequency energy channel designations. In the figure, the electrode supporting uppermost flexible circuits of the implants or wands 1-3 are represented respectively at 410-412. These flex circuits are described, for example, at FIGS. 12 and 13 at 100. The embedded resistor segment carrying circuit as described in conjunction with FIGS. 14 and 15 at 88 are represented respectively at blocks 414-416. The gold-plated copper electrodes at circuit 410 of implant number 1 are represented in general at 418 and are identified as E1-A-E1-D. Correspondingly, flex circuit 411 supports four radiofrequency electrodes represented generally at 420 which are identified as E2-A-E2-D and flex circuit 412 supports four radiofrequency electrodes represented generally at 422 and identified as E3-A-E3-D. Electrode arrays 418, 420 and 422 correspond, for example, with electrodes 104-107 illustrated in FIG. 12. Electrodes 418 are seen to be operationally coupled by leads extending to lead contacts represented generally at 424 and identified as L1F-A-L1F-D. Similarly, electrodes of array 420 are coupled by leads to lead contacts represented generally at 425 and identified as L2F-A-L2F-D; and the electrodes of array 422 are coupled by leads extending to lead contacts represented generally at 426 and identified as L3F-A-L3F-D. The lead structure of blocks 410-412 correspond with leads 170-173 described in connection with FIGS. 12 and 13. Contacts 424 are seen to be operationally associated by a line array represented generally at 428 with a corresponding array of four output channels represented generally at 430. These output channels identify the bipolar association between lead contact arrays 424 and 425. In this regard, they are identified as CH1-2A-CH1-2D. Such channels have been described in FIG. 25 at block 384. Four channel array 430 additionally is operationally associated with lead contact array 425 of implant number 2 by a lead line array represented in general at 432. For instance, output channel CH1-2A provides a bipolar energization association between contact lead L1F-A of array 424 and contact lead L2F-A of contact lead array 425. The bipolar energy association between electrodes E1-A-E1-D and respective electrodes E2-A-E2-D are represented by the R.F. energy transfer symbols identified generally at 434.

In similar fashion, the contact leads of array 426 of implant number 3 are operationally associated with a corresponding array of four radiofrequency output channels represented generally at 436 by a line array represented generally at 438. In this regard, lead contacts L3F-A-L3F-D are operationally associated with respect to output channels CH2-3A-CH2-3D. As represented by the line array identified generally at 440, the four radiofrequency output channels 436 are operatively associated in bipolar fashion with the corresponding contact leads 425 of implant number 2. In this regard, channels CH2-3A-CH2-3D are associated in bipolar relationship with contact leads L2F-A-L2F-D This bipolar association provides for electrode-to electrode R.F. energy transfer as represented by the energy transfer symbols identified in general at 442.

Looking to the embedded flexible circuit assemblies 414-416 of respective implant numbers 1-3, three arrays of temperature sensing resistors are identified generally at 450-452. Sensing resistor arrays 450-452 are coupled by a four-point configured lead array extending to seven lead contacts identified in general respectively at 454-456. Resistor arrays as at 450-452 have been described in connection with FIG. 14 at 92-95, while lead contact arrays have been described in conjunction with FIG. 15 at 180-186. The four temperature feedback interface channels represented at contact lead array 454 are illustrated as being associated with a resistive feedback monitor function or channels 1-4 at block 458 by the line array represented generally at 460. In similar fashion, the four channels represented by contact lead array 455 are operationally associated with resistant feedback monitor channels 5-8 as represented at block 462 and the line array identified generally at 464. The four sensing channels represented by four resistor array 452 and contact lead array 456 are associated with resistant feedback monitor for channels 9-12 as represented by block 466 and the line array identified generally at 468.

Studies have been carried out to model and theorize the heat transfer phenomena associated with the instant system and method, particularly with respect to epidermal over-temperatures and the diversion of current across muscle as illustrated at 270 in connection with FIG. 20. The latter effect of R.F. current channeling through the underlying muscle appears to have occurred in situations in which the subcutaneous fat layer is thin. (Accordingly, mechanical pressure applied using the earlier skin temperature control units in the form of a water-filled polyethylene bag may exacerbate this problem.) As may be seen in Table 1, the electrical resistivity in porcine muscle is about one third that of dermis. Additionally, since the dermis is only on the order of 1 mm to 2 mm thick while the next adjacent muscle layer can typically be 5 mm or greater in thickness, the combined effect of lower electrical resistivity and greater thickness can result in an electrical resistance in the muscle layer which is at least five times lower than that of the dermis. Consequently, the thickness of the very high resistivity fat layer as summarized in Table 1 plays a critical role in limiting the channeling of R.F. current into muscle.

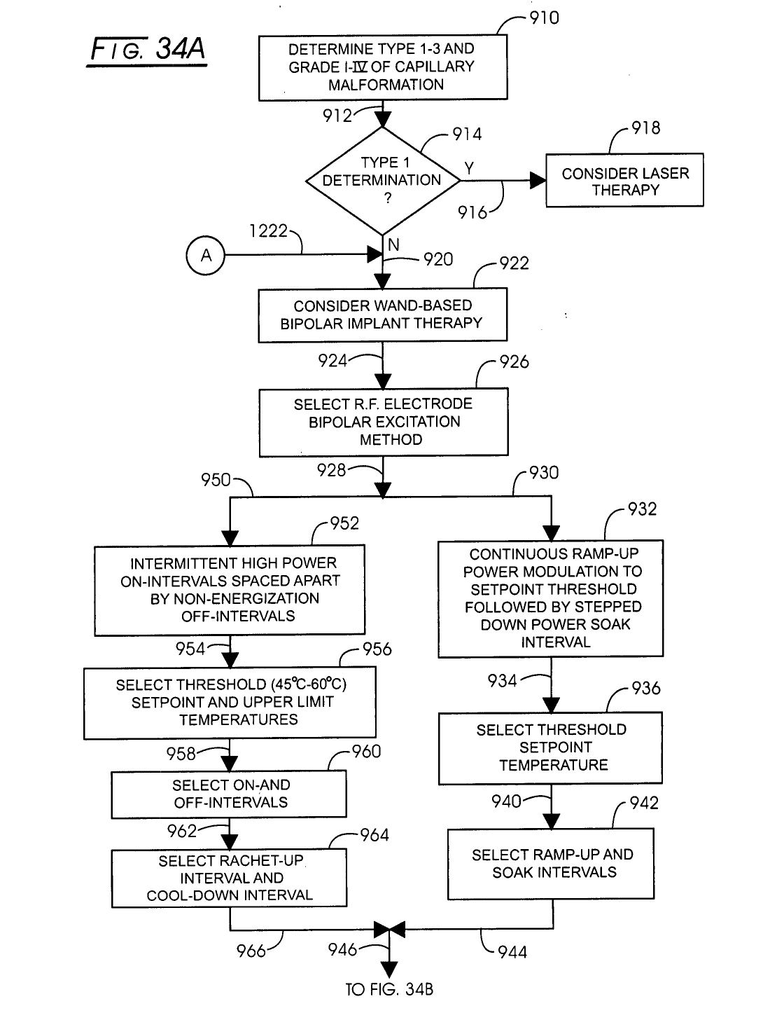

To facilitate the analysis to follow, a schematic section of skin is provided in FIG. 27. In that figure, epidermis is represented at 480, the drawing also showing rete ridges as at 482. The thickness of epidermis 480 is considered to include those ridges 482 and the epidermis/dermis boundary occurs at that level of the skin section. Dermis is represented at 484 and the next subcutaneous tissue or fat layer is represented at 486. The interface between fat layer 486 and dermis 484 is represented at 488. Two single electrode implants or wands are located in heating channels at the interface 488. As described in conjunction with FIGS. 4 and 5, these implants are 3 mm in width and are arranged in parallel relationship at a 15 mm center-to-center spacing. Accordingly, the total heated width involved in this demonstration is 18 mm. The electrodes of implants 490-492 having an effective length of 15 mm, a total heated area involved is 15 mm×18 mm. R. F. current flow is represented schematically by the dashed line shown generally at 494.

The thermal analysis of the skin seeking to calculate maximum temperature of the epidermis, which occurs at the epidermis/dermis interface, involves conduction heat transfer in accordance with the established equation:

Q=(k*A*DT)/L (3)

where Q is the amount of heat conducted in watts; k, is the thermal conductivity of the medium in watts/cm-C; A is the area through which conduction is occurring (in cm²); DT, is the temperature difference across the medium in which conduction heat transfer is occurring (° C.); and L is the length over which heat is conducted. For the present analysis, emphasis is upon the total temperature difference, DL, across the epidermis which provides an estimation of the maximum temperature at the epidermis/dermis interface or at the high side of the thermal gradient.

In considering FIG. 27, the total power involved for the demonstration, Qₜₒₜₐₗ, will be known with respect to both the intermittent mode of performance and the continuous mode of performance. For example, it will range from about 10 to about 14 watts. Some portion of that total will be conducted through the epidermis 480 as represented symbolically at 496. The remaining heat will be conducted into deeper tissue as represented by the symbols 498. A heat conduction relationship then can be expressed as follows:

Qₜₒₜₐₗ=Q_{conduction into deeper tissue}+Q_{conduction through epidermis} (4)

What is unknown is the split or relationship between conduction paths 496 and conduction paths 498. Some reasonable estimations can be made. For example, conduction through the epidermis as at 496 increases as the dermis 484 becomes thinner because the conduction pathway is shorter. It is estimated based on the thermal impedance of the fat layer and the cooling effect of blood perfusion in the underlying muscle layer that at least 50% to 60% of the R.F. power dissipated between electrodes as at 490 and 492 flows through the epidermis to the skin surface. With respect to heat conducting through the epidermis as at 496, an estimate can be made based upon a knowledge of when burning does not occur. In this regard, between about 6 to about 7 watts for continuous mode heating may be estimated and between about 7 and about 9 watts may be estimated for the intermittent mode of performance. However, in the latter mode it may be recalled that the epidermis is re-cooled to a sufficiently low temperature at the end of each brief heating cycle as discussed in connection with FIG. 21.

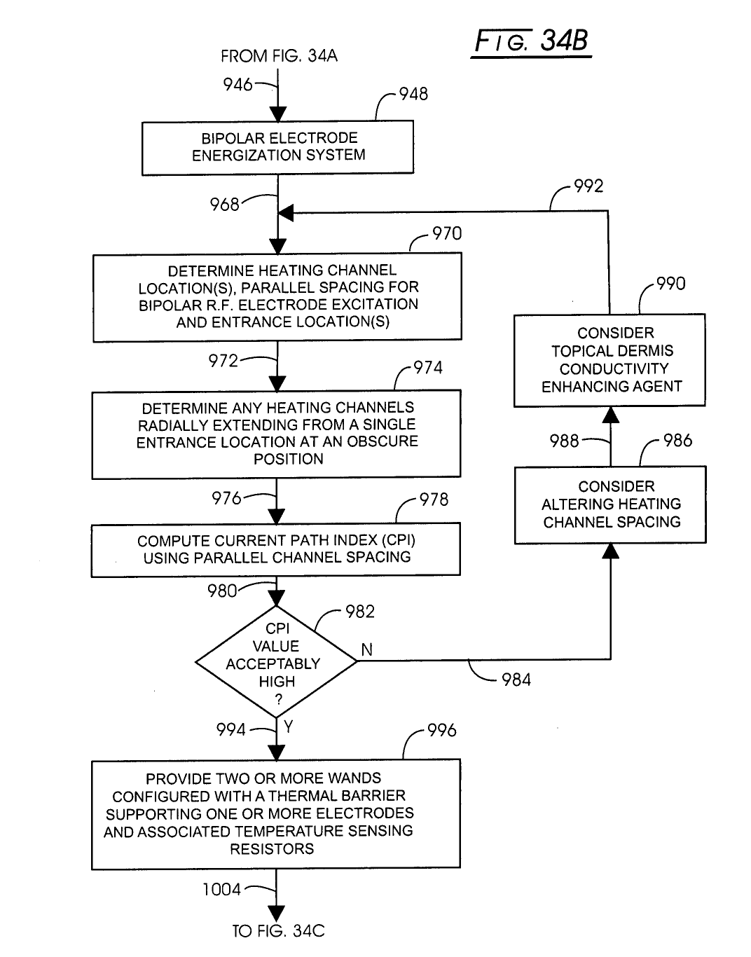

Looking to FIG. 28, three curves, 502-504 are provided relating maximum temperature rise at the epidermis/dermis boundary in degrees centigrade with respect to an area of heat conduction which is 15 mm×18 mm. Curves 502-504 respectively represent temperature rise through epidermis thicknesses of 0.20 mm, 0.15 mm, and 0.08 mm. This range of thicknesses was selected based on actual measurements of porcine epidermis thickness carried out at The Ohio State University Medical Center as well as published values of epidermis thickness. To avoid epidermis burn, the noted interface should not exceed 45° C. to about 47° C. Curve 502 indicates that a 25° C. temperature difference will correspond with the 7 watts of heat flow. Accordingly, with a 45° C. limit the surface must be maintained at 20° C. or less to avoid a burn.

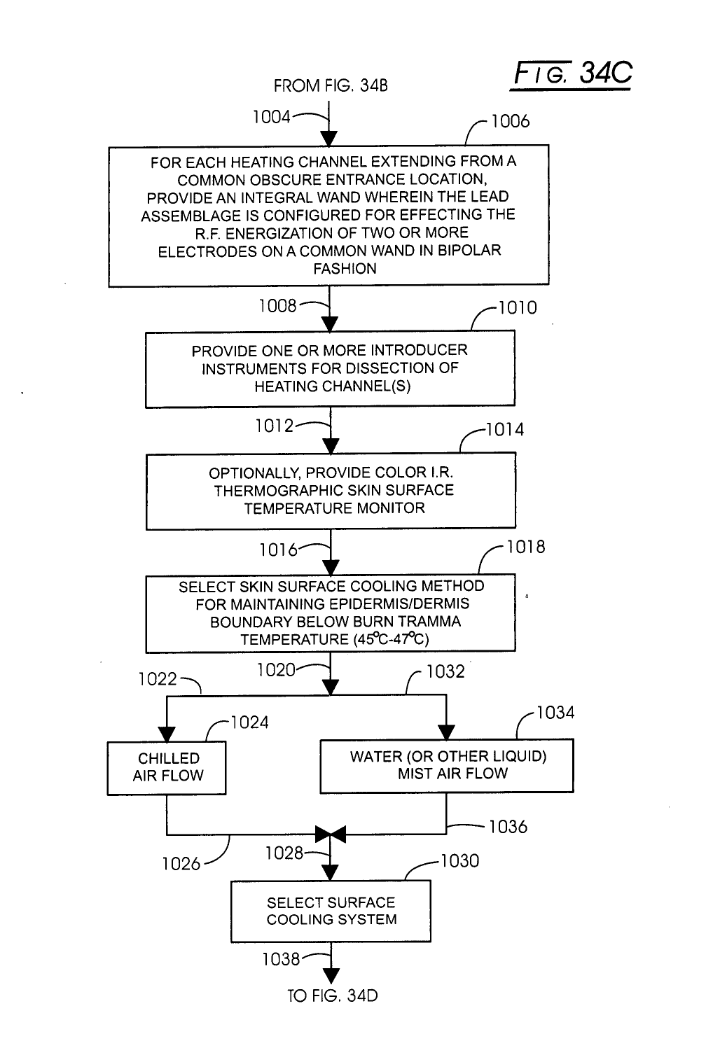

Referring to FIG. 29, an epidermis thickness of 0.15 mm is assumed and curves 506-508 were plotted with respect to respective surface temperatures of skin of 25° C., 20° C. and 17° C. Accordingly, plots 506-508 provide a parameter which is useful inasmuch as it relates what the epidermis/dermis boundary temperature can rise to as a function of maximum skin surface temperature. For example, plot 506 indicates that at an interface temperature of about 45° C., as much as 8 watts of heat will be conducted to the surface of the epidermis. Clinicians will probably want to maintain the surface temperature at 20° C. or below for a maximum of 8 watts of power being conducted through epidermis.

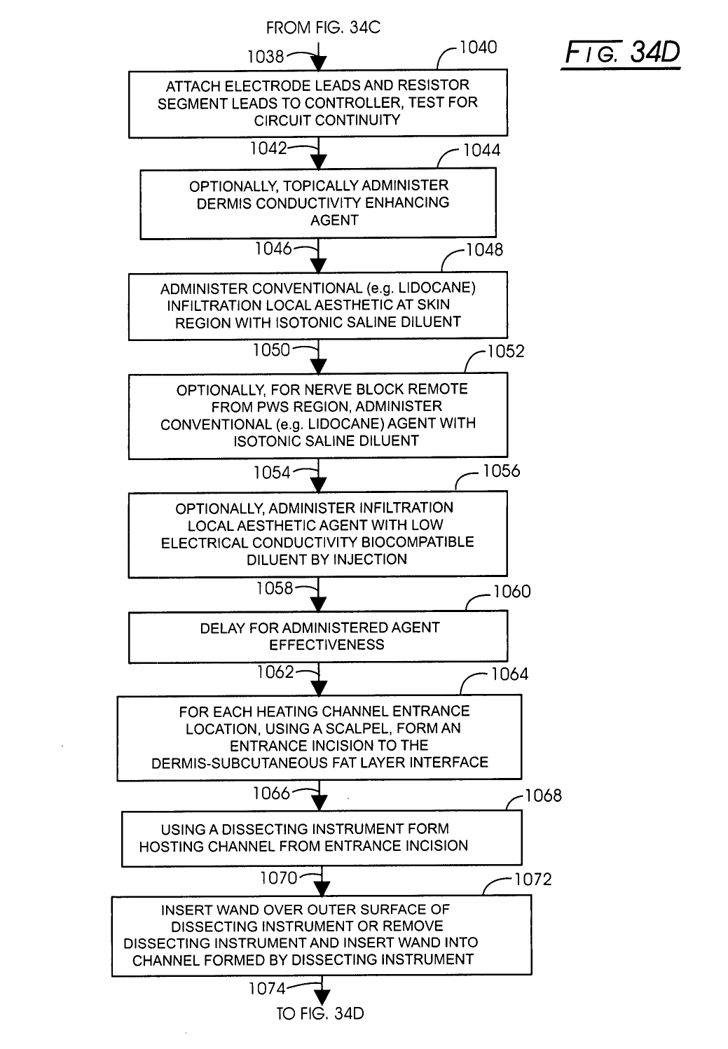

Looking to FIG. 30, plots 510-512 are provided again with respect to maximum epidermis skin surface temperatures respectively of 25° C., 20° C. and 17° C. as in the case of FIG. 29. However, an epidermis thickness of 0.20 mm is assumed.

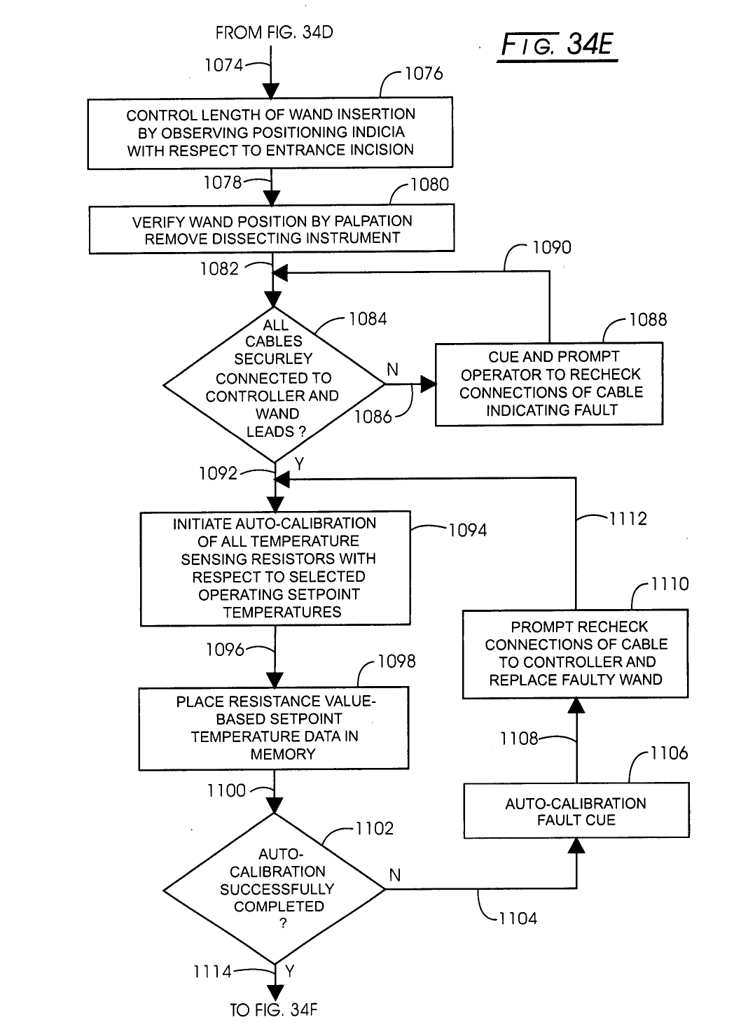

Referring to FIG. 31, the same form of data as provided in connection with FIGS. 29 and 30 is presented at plots 514-516. However, epidermis thickness is assumed to be 0.08 mm. As before, plots 514-516 respectively represent skin surface maximum temperatures respectively of 25° C., 20° C., and 17° C.

As discussed in connection with FIG. 20, histopathology investigation associated with animal studies reveals that there are circumstances wherein the R.F. current can flow through muscle to an extent causing coagulative necrosis, a clinically unacceptable condition. Harkening back to Table 1, a tabulation of electrical resistivity of dermis, subcutaneous fat and muscle is set forth. The tabulation reveals that dermis exhibits a resistivity of 263 to 270 ohm centimeters while subcutaneous fat exhibits a resistivity almost eight times greater and the resistivity of muscle is quite low. However, with respect to R.F. current flow resistance with its volumetric aspects must be considered. In general, resistance is equal to resistivity times length divided by area. Applying that relationship to the assumed geometry of FIGS. 20 and 27, the following relationship obtains:

$$R = \frac{ρ L}{A} = ρ \frac{( {I}_{nterelectrode} {S}_{pacing} )}{( {L}_{electrode} * {t}_{dermis} )} \qquad ( 5 )$$

Accordingly, if muscle is assumed to be 4 mm thick, dermis is assumed to be 1 mm thick, the resistance of muscle will be one eighth that of dermis because of its factor of four increase in thickness. As described in connection with FIG. 20, diversion of R.F. current from electrodes 264 and 266 into muscle layer 262 also can be occasioned by conduction through certain fibrous septae as represented at 272 and 274.

While histopathology tests have shown that R.F. current damage can occur at the muscle layer, it does so rarely. Such damage indicates that the temperature reached over the time of treatment was at about 55° C. or over.

Upon examination of the factors described above which influence the current path between implant or wand carried electrodes a theory and model was developed for purposes of predicting when a significant level of current could flow through the muscle layer. These factors are illustrated and identified in FIG. 20 and their relationship may be employed to develop what is referred herein as a “current path index” (CPI).

The thickness of the dermis, t_{D}, is one of the factors determining current flow via alternative pathways due to the fact that the electrical resistance of the dermis is directly proportional to its thickness. Hence, a dermis layer 1 mm thick will represent twice as much resistance to electrical current flow as a dermis layer which is 2 mm thick. As a consequence, the thinner the dermis layer, the greater the possibility that R.F. current might flow between the implant or wand electrodes via some alternative pathway. This possibility of alternative pathway however requires that the electrical resistance along the alternative pathway be comparable to or less than the electrical resistance in the dermis layer pathway.

The thickness of the subcutaneous fat layer, t_{SF}, is another factor which determines current flow via alternative pathways due to the fact that the electrical resistance of the fat layer in a pathway from the implant or wand electrode to the muscle layer is directly proportional to the thickness of the fat layer. Due to the much higher electrical resistivity of subcutaneous fat as compared to dermis, it may be hypothesized that R.F. current flow through the fat layer occurs predominately via the much more conductive fibrous septae. Hence, a subcutaneous fat layer 3 mm thick will represent twice as much resistance to electrical current flow as a subcutaneous fat layer which is 6 mm thick. In this model, it is assumed that any alternative current path via the muscle layer will involve current flow through the shortest possible distance between the subcutaneous fat layer and the much lower electrical resistance pathway associated with the muscle layer. As illustrated in FIG. 20, the shortest possible distance between the electrodes and the muscle layer is approximately equal to the thickness of the subcutaneous fat layer.

The centerline spacing between the wand or implant electrodes, S_{E}, is the third factor which determines current flow via alternative pathways due to the fact that (a) depending on the level of hydration, the electrical resistivity of the dermis is approximately twice as large as that of the muscle layer, and (b) the muscle layer thickness may be about four times or more greater than the thickness of the dermis. If the dermis is poorly hydrated, it is hypothesized that the difference in electrical resistivity between the dermis and muscle layer may even be greater than two times. As a consequence, the resistance to electrical current flow within the dermis increases proportionally with interelectrode spacing, S_{E}. Since the absolute level of electrical resistance of the muscle layer is much less than that of the dermis layer, its proportional increase with interelectrode spacing, S_{E}, still represents a lower pathway resistance than the dermis layer. Hence, the critical factor which determines how much current will flow via the muscle is not its electrical resistance but rather the electrical resistance of the alternative pathway through the subcutaneous fat layer as compared with the electrical resistance of the dermis layer.

The present model is developed for assessing the possibility of clinically significant current flow at the muscle layer. The model assigns equally to the three factors discussed above to obtain a dimensionless current path index value (CPI). The current path index is calculated ratiometrically using assumed reference values for each of the three parameters as follows:

$$CPI = \frac{( {t}_{D} / 2 mm ) * ( {t}_{SF} / 5 mm )}{{S}_{E} / 15 mm} \qquad ( 6 )$$

which simplifies to:

$$CPI = \frac{1.5 * {t}_{D} * {t}_{SF}}{{S}_{E}} \qquad ( 7 )$$

where
t_{D}=thickness of the dermis (in mm)
t_{SF}=thickness of the subcutaneous fat layer (in mm)
S_{E}=centerline spacing between electrodes (in mm)

The rationale for Equation (6) is based on the discussion above for each of these three factors. First, the larger the dermis thickness, t_{D}, the lower its electrical resistance and the greater the propensity for R.F. current flow through the dermis. Likewise, the larger the thickness of the subcutaneous fat layer, t_{SF}, the greater the propensity for R.F. current flow path through the dermis and not through the subcutaneous fat layer to the muscle layer since the current flow path through the subcutaneous fat layer is proportional to its thickness, t_{SF}. Both of these factors are assumed to be positively correlated with the (preferred) current flow path through the dermis. Hence, both of these factors are in the numerator of the Current Path Index, CPI in Equation (6). In contrast, the interelectrode spacing, S_{E}, is in the denominator of the current path index in Equation (7). In contrast, the interelectrode spacing, S_{E}, is negatively correlated with current flow through the dermis since the greater the interelectrode spacing, the greater the likelihood that R.F. current will flow through the muscle layer. As a consequence, the interelectrode spacing S_{E}, is in the denominator of the current path index equation.

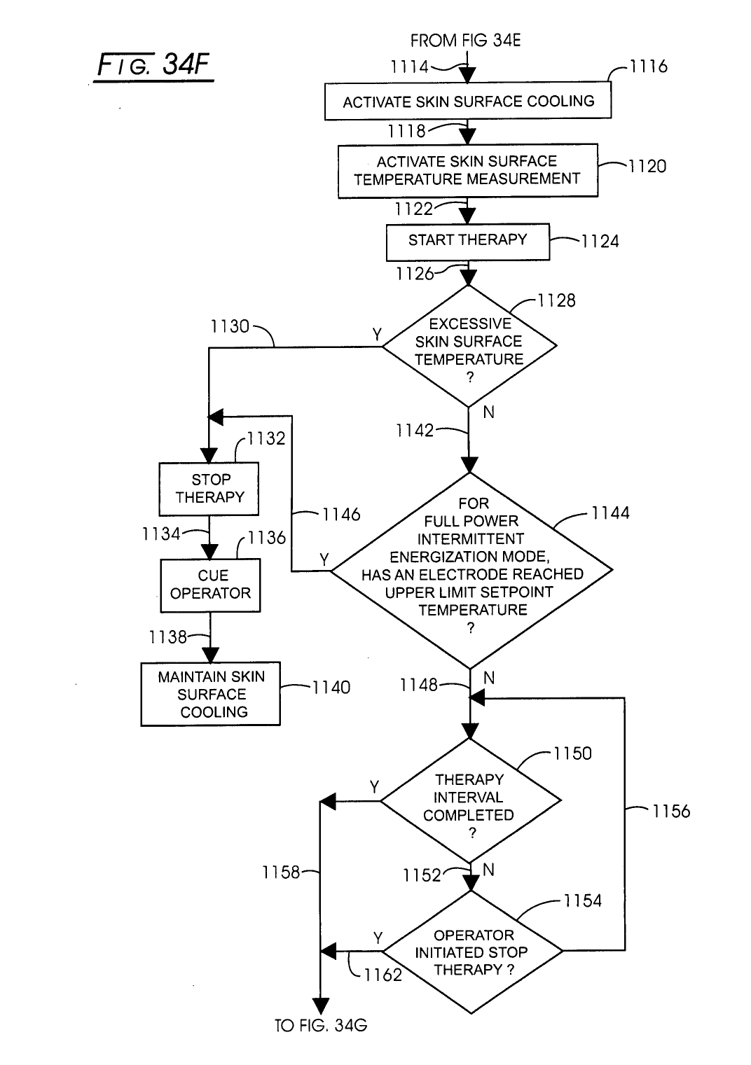

The R.F. current flow model is based on the assumption that, at some value of current path index, CPI, there will be evidence (based on histopathology analysis of tissue in the treatment zone) of current flow in the muscle layer. This hypothesized model has been tested by examining the histopathology findings obtained regarding the presence and depth of acute coagulative damage within the muscle layer. CPI was computed with respect to histopathology reports stemming from in vivo animal (pig) experiments. In this regard, a scatter diagram of the depth of acute coagulative damage as a function of current path index is presented in FIG. 32. As seen in this diagram, a measurable depth of acute coagulative damage has not been observed at current path index values greater than about 0.61 based on a total of 29 data points. The only three occurrences of acute coagulative damage in the muscle layer were for current path index values of 0.57 or less.

The above analysis leads to a further observation that maintenance of R.F. current flow within the dermis can be enhanced by elevating the conductivity or lowering resistivity of dermis. In this regard, a topical agent effective for reducing electrical resistivity of the target tissue can be applied, for instance, to the surface of the treated region. Such an agent, a dermis conductivity enhancing agent, will preferably act to decrease the resistivity of the dermis by providing a mechanism to increase current flow through that tissue relative to the current flow in subadjacent tissues. Muscle tissue has relatively low resistivity due to the presence of ionic components that are capable of carrying currents. A relative decrease in the resistivity of the dermis compared to the low resistivity of the adjacent muscle tissue is predicted to increase the current flow through the dermis and increase the heating of dermis tissue relative to other adjacent tissues. Dermis conductivity enhancing agents include such agents as metal ions, such as calcium, magnesium, sodium and potassium, and also substances or treatments that lead to the release of electrically conductive substances from the dermal tissues, such as enzymes or electrical or thermal shock. Dermis conductivity enhancing agents may be delivered to the skin surface, injected into the dermis, or released from the surface of the inserted implants. It should be noted that because the subadjacent muscle layer already possesses high electrical conductivity, if the conductivity of the dermis is increased, while also effecting an increased conductivity in the muscle layer, dermis conductivity enhancement will still result, because the CPI of the muscle layer will not increase sufficiently to lead to additional heating of the muscle layer, while the heating of the dermis will be enhanced due to the relative increase of the relatively low dermal conductivity.

Threshold setpoint temperatures have been discussed in connection with FIGS. 19 and 21 as being the entry level temperatures required to induce collagen shrinkage. Typically, those threshold values will be between about 63° C. and 73° C. These temperatures might be referred to as thermal transformation temperatures. A number of substances have been identified that interact with the ECM of the dermis to alter the thermally responsive properties of the collagen fibers. As described herein, substances with such properties are termed “adjuvants”. It will be recognized by those skilled in the art of protein structural chemistry that the reduction in length of collagen fibers, i.e., shrinkage, is the result in part of an alteration of the physical structure of the molecular structure of the collagen fibers. The internal ultrastructure of collagen fibers, being comprised of tropocollagen molecules aggregated into collagen fibrils, and then aggregated further into even larger collagen fibers, is a result of complex interactions between the individual tropocollagen molecules, and between molecules associated with the collagen fibers, for example, elastin, and hyaluronan. The molecular forces of these interactions include covalent, ionic, disulphide, and hydrogen bonds; salt bridges; hydrophobic, van der Waals forces. In the context of the present disclosure, adjuvants are substances that are capable of inducing or assisting in the alteration of the physical arrangement of the molecules of the skin in order to induce, for instance shrinkage. With respect to collagen fibers, adjuvants are useful for altering the molecular forces including those hydrophilic and hydrophobic forces holding collagen and associated molecules in position, changing the conditions under which shrinkage of collagen can occur.

Protein molecules, such as collagen are maintained in a three dimensional arrangement by the above described molecular forces. The temperature of a molecule has a substantial effect on many of those molecular forces, particularly on relatively weaker forces such as hydrogen bonds. An increase in temperature may lead to thermal destabilization, i.e., melting, of the three dimensional structure of a protein. The temperature at which a structure melts is known as the thermal transformation temperature. In fact, irreversible denaturation of a protein, e.g., cooking, is a result of melting or otherwise disrupting the molecular forces maintaining the three dimensional structure of a protein to such an extent that that once heat is removed, the protein can no longer return to its initial three dimensional orientation. Collagen is stabilized in part by electrostatic interactions between and within collagen molecules, and in part by the stabilizing effect of other molecules serving to cement the molecules of the collagen fibers together. Stabilizing molecules may include proteins, polysaccharides (e.g., hyaluronan, chondroitin sulphate), and ions.

A persistent problem with existing methods of inducing collagen shrinkage that rely on heat is that there is a substantial risk of damaging and or killing adipose (fat layer) tissue underlying the dermis, resulting in deformation of the contours of the overlying tissues, with a substantial negative aesthetic effect. Higher temperatures or larger quantities of energy applied to the living cells of the dermis can moreover result in irreversible damage to those cells, such that stabilization of an altered collagen network cannot occur through neocollagenesis. Damage to the living cells of the dermis will negatively affect the ability of the dermis to respond to treatment through the wide variety of healing processes available to the skin tissue. Adjuvants that lower the thermal transition temperature required for shrinkage have the advantage that less total heat need be applied to the target tissue to induce shrinkage, thus limiting the amount of heat accumulating in the next adjacent subcutaneous tissue layer (hypodermis). Reducing the total energy application is expected to minimize tissue damage to the sensitive cells of the hypodermis, thereby limiting damage to the contour determining adipose cells.

One effect of such adjuvants is that certain chosen biocompatible reagents have the effect of lowering the temperature required to begin disruption of certain molecular forces. In essence, adjuvants are capable of reducing the molecular forces stabilizing the ultrastructure of the skin, allowing a lower absolute temperature to induce shrinkage of the collagen network that determines the anatomy of the skin. Any substance that interferes with the molecular forces stabilizing collagen molecules and collagen fibers will exert an influence on the thermal transformation temperature (melting temperature). As collagen molecules melt, the three dimensional structure of collagen undergoes a transition from the triple helix structure to a more random polypeptide coil. The temperature at which collagen shrinkage begins to occur is that point at which the molecular stabilizing forces are overcome by the disruptive forces of thermal transformation. Collagen fibers of the skin stabilized in the ECM by accessory proteins and compounds such as hyaluronan and chondroitin are typically stable up to a temperature of approximately 58° C. to 60° C., with thermal transformation and shrinkage occurring in a relatively narrow phase transition range of 60-70° C. Variations of this transition range are noted to occur in the aged (increasing the transition temperature) and in certain tissues (decreasing by 2-4° C. in tendon collagen). In effect the lower temperature limit of the collagen shrinkage domain is determined by the thermal transformation temperature of a particular collagen containing structure.

It will be recognized by those skilled in molecular biology that the thermal transformation temperature necessary to achieve a reduction in skin laxity may not entirely be determined by the thermal transformation temperature of collagen fibers, but may also be affected by a variety of other macromolecules present in the dermis, including other structural proteins such as elastin, fibronectin, heparin, carbohydrates such as hyaluronan and other molecules such as water and ions.

Referring again to FIG. 19, a hypothetical plot or curve 520 showing desired electrode temperature with respect to therapy duration is presented wherein an adjuvant is used along with the implants. In the figure, a starting temperature is shown again to be, for example, 33° C. Above that temperature between about 51° C. and 61° C., when an adjuvant lowering the thermal transition temperature by 12° C. is present, there is established a collagen shrinkage domain represented generally at 522. Shrinkage domain 522 is seen to extend between the dashed line level 524 corresponding with a collagen shrinkage threshold temperature of 51° C. and dashed line level 526 corresponding with an upper limit level temperature of about 61° C. As represented previously at electrode temperature versus time curve portion 228, variable power is applied to the bipolar electrodes as a ramp control commencing at the noted 33° C. and reaching the upper limit of 61° C. within domain 522 at position 528 corresponding with a controlled therapy ramp interval of about four minutes. At about position 528, power input to the electrodes is reduced and, as represented by curve portion 530 a reduced power input is provided with constant power control for about a two minute interval, for example, between the fourth and sixth minutes to evoke the previously noted “thermal soak”.

Substances exhibiting the properties desirable for lowering the thermal transition temperature include enzymes such as hyaluronidase, collagenase and lysozyme; compounds that destabilize salt bridges, such as beta-napthalene sulphuric acid; each of which is expected to reduce the thermal transition temperature by 10-12° C., and substances that interfere with hydrogen bonding and other electrostatic interactions, such as ionic solutions, such as calcium chloride or sodium chloride; detergents (a substance that alters electrostatic interactions between water and other substances), such as sodium dodecyl sulphate, glycerylmonolaurate, cationic surfactants, or N,N, dialkyl alkanolamines (i.e. N,N-diethylethanolamine); lipophilic substances (lipophiles) including steroids, such as dehydroepiandrosterone, and oily substances such as eicosapentanoic acid; organic denaturants, such as urea; denaturing solvents, such as alcohol, ethanol, isopropanol, acetone, ether, dimethylsulfoxide (DMSO) or methylsulfonylmethane; and acidic or basic solutions. The adjuvants that interfere with hydrogen bonding and other electrostatic interactions may reduce the thermal transition temperature by as much as 40° C. depending on the concentration and composition of the substances administered. The extent of effectiveness of a particular adjuvant in use will be dependent on the chemical properties of the adjuvant and the concentration of adjuvant administered to the patient. For enzymatic adjuvants such as hyaluronidase, the thermal transition temperature is also dependent on the specific activity of the delivered enzyme adjuvant in the dermis environment.

Adjuvants suitable for use would desirably be compatible with established medical protocols and be safe for use in human patients. Adjuvants should be capable of rapidly infiltrating the targeted skin tissue, should cause minimal negative side effects, such as causing excess inflammation, and should preferably persist for the duration of the procedure. Suitable adjuvants may be, for instance, combined with local anesthetics used during treatment, be injectable alone or in combination with other reagents, be heat releaseable from the implants of the invention, or be capable of entering the targeted tissue following topical application to the skin surface. Certain large drug molecules, such as enzymes functioning as adjuvants according to the invention may be drawn into the target dermal tissue through iontophoresis (electric current driving charged molecules into the target tissues) The exact mode administration of adjuvants will be dependent on the particular adjuvant employed.

In a preferred embodiment, the thermal transition temperature lowering adjuvant is present in highest concentrations in the tissues of the dermis. For highest efficacy, a concentration gradient is established, wherein the adjuvant is at a higher concentration in the dermis that in the hypodermis. A transdermal route of administration is one preferred mode of administration, as will occur with certain topical adjuvants. For adjuvants that are applied topically to the surface of the skin, for instance as a pomade, as the adjuvant either diffuses or is driven across the epidermis, and passes into the dermis, a concentration gradient is established wherein the adjuvant concentration is higher in the dermis than in the hypodermis. Because the collagen matrix is much more prevalent in the dermis than in the epidermis, presence of the adjuvant in the epidermis is expected to be without negative effect. Certain adjuvants, for instance, enzymes with collagen binding activity, would be expected to accumulate in the dermal tissue.

A variety of methods are known wherein drugs are delivered to the patient transdermally, i.e. percutaneously, through the outer surface of the skin. A variety of formulations are available that enhance the percutaneous absorption of active agents. These formulations may rely on modification of the active agent, or the vehicle or solvent carrying that agent. Such formulations may include solvents such as methylsulfonylmethane, skin penetration enhancers such as glycerylmonolaurate, cationic surfactants, and N,N, dialkyl alkanolamines such as N,N-diethylethanolamine, steroids, such as dehydroepiandrosterone, and oily substances such as eicosapentanoic acid. For further discussion of enhancers of transdermal delivery of active agents, for instance adjuvants according to the invention, see: U.S. Pat. No. 6,787,152 to Kirby et al., issued Sep. 7, 2004; and U.S. Pat. No. 5,853,755 to Foldvari, issued Dec. 29, 1998.

When adjuvants are injected, it is preferable that they be deposited as close to the dermis as practicable, preferably, intradermally. Because the dermis is relatively thin, and difficult to penetrate with hypodermic needles, the invention is also embodied in adjuvants that are delivered subdermally, or at the interface between the dermis and the next adjacent subcutaneous tissue (hypodermis or adipose tissues underlying the dermis). Even to the extent that adjuvants are delivered into the adipose tissue of the hypodermis, because the hypodermis is typically very thick compared to the dermis, a concentration gradient will develop, wherein the adjuvant will diffuse quickly into the dermis, and fully equilibrate with the dermal tissue, before the adjuvant has fully equilibrated with the hypodermis.

In a further embodiment, the implants carry a surface coating of adjuvant that is released into the dermis upon activation of the implant. It is an advantage of the invention when utilizing thermal transition temperature lowering adjuvants that the implants are placed very near the location where adjuvants can provide the most benefit. A number of compositions are known in the art that can be released from an implant by heating of the implant. For example, the upper, or dermis facing, surface of the implant can be coated with microencapsulated adjuvant, for instance hyaluronan. Once a preliminary heating of the implant begins, the encapsulated adjuvant is released, and immediately begins diffusing into the dermis tissue, as the implant is already in place at the interface between the dermis and hypodermis. As the adjuvant diffuses through the dermis, a concentration gradient develops wherein the adjuvant is at the greatest concentration in the dermis, with reduced concentrations in the epidermis and hypodermis. Following this preliminary heating, regular ramp up to a lowered setpoint temperature may be carried out. As described previously, while it is not a requirement that the adjuvant be at greatest concentration in the dermis (for instance, if the adjuvant is applied topically to the skin surface), it is considered an advantage to for the adjuvant to be at the greatest concentration in the tissue layer wherein adjuvant activity is needed.

In a further embodiment of implant delivery of the adjuvant, the adjuvant is encapsulated in liposomes and suspended in a compatible vehicle. The surfaces of the implant to be inserted into the patient are then coated with the liposome/vehicle composition. When the implant is inserted into the tissue of the patient, the vehicle coating, preferably moderately water soluble and biologically inert, prevents the adjuvant from being displaced from the implant surface for the period of time necessary for insertion. Once the implant is activated on the noted preliminary basis, the dermis facing upper surface of the implant is heated and the liposomes encapsulating the adjuvant are induced by heat to release the adjuvant. The adjuvant may alternatively be released from implants by brief preliminary heating. Different compositions of liposomes are useful for providing release of the adjuvant at a particular temperature range. Similarly, the vehicle binding the adjuvant encapsulating liposomes to the implant can be chosen so that the vehicle does not release the liposomes themselves unless a desired temperature has been reached. In this manner the release of adjuvant from an implant surface may be configured so that the adjuvant is released in a directional manner, even though the entire implant surface is coated with an adjuvant composition. Those skilled in the art will recognize that a variety of heat releaseable encapsulating systems are available for use with the invention. Further discourse on the composition of liposomes is available by referring to U.S. Pat. No. 5,853,755 (supra).

The following discourse specifically describes certain embodiments of specific adjuvants that are useful. Artisans will recognize that other substances known in the art to have similar effects will be useful as adjuvants, and thus, the following embodiments should not be considered as limiting.

Hyaluronidase is an enzyme that cleaves glycosidic bonds of hyaluronan, depolymerizing it and, converting highly viscous polymerized hyaluronan into a watery fluid. A similar effect is reported on other acid mucopolysaccharides, such as chrondroitin sulphate. Hyaluronidase is commercially available from a number of suppliers (e.g., Hyalase, C. P. Pharmaceuticals, Red Willow Rd. Wrexham, Clwydd, U.K.; Hylenex, Halozyme Therapeutics (human recombinant form); Vitrase, (purified ovine tissue derived form) ISTA Pharmaceuticals; Amphadase, Amphastar Pharmaceuticals (purified bovine tissue derived)).

Hyaluronidase modifies the permeability of connective tissue following hydrolysis of hyaluronan. As one of the principal viscous polysaccharides of connective tissue and skin, hyaluronan in gel form, is one of the chief ingredients of the tissue cement, offering resistance to the diffusion of liquids through tissue. One effect of hyaluronidase is to increase the rate of diffusion of small molecules through the ECM, and presumably to decrease the melting temperature of collagen fibers necessary to induce shrinkage. Hyaluronidase has a similar lytic effect on related molecules such as chondroitin sulphate. Hyaluronidase enhances the diffusion of substances injected subcutaneously, provided local interstitial pressure is adequate to provide the necessary mechanical impulse. The rate of diffusion of injected substances is generally proportionate to the dose of hyaluronidase administered, and the extent of diffusion is generally proportionate to the volume of solution administered. The addition of hyaluronidase to a collagen shrinkage protocol results in a reduction of the thermal transition temperature required to induce 20% collagen shrinkage by about 12° C. Review of pharmacological literature reveals that doses of hyaluronidase in the range of 50-1500 units are used in the treatment of hematomas and tissue edema. Thus, local injection of 1500 IU hyaluronidase in 10 ml vehicle into the target tissue is predicted to reduce the temperature necessary to accomplish 20% shrinkage of collagen length from about 63° C. to about 53° C. For multiple injection sites 100 IU hyaluronidase in 2 ml of alkalinized normal saline or 200 IU/ml are expected to be similarly effective as an adjuvant. The manufacturer's recommendations for Vitrase indicate that 50-300 IU of Vitrase per injection are expected to exert the adjuvant effect. It should be noted that use of saline vehicle for delivery of adjuvants and anesthesia may be contraindicated where introduction of excess electrolytes would interfere with operation of the implants.

Hyaluronidase has been used in clinical settings as an adjunct to local anesthesia for many years, without significant negative side effects, and is thus believed to be readily adaptable for use with the instant method. When used as an adjunct to local anesthesia, 150 IU of hyaluronidase are mixed with a 50 ml volume of vehicle that includes the local anesthetic. A similar quantity of hyaluronidase is expected to be effective for reducing the thermal transition temperature for effecting shrinkage by approximately 10° C., with or without the addition of anesthetic. When hyauronidase is injected intradermally or peridermally, the dermal barrier removed by hyaluronidase activity persists in adult humans for at least 24 hours, with the permeabilization of the dermal tissue being inversely related to the dosage of enzyme delivered (in the range of administered doses of 20, 2, 0.2, 0.02, and 0.002 units per mL. The dermis is predicted to be restored in all treated areas 48 hours after hyaluronidase administration. Additional background on the activity of hyaluronidase is available by referring to the following publications (and the references cited therein):
21. Lewis-Smith, P. A., “Adjunctive use of hyaluronidase in local anesthesia” Brit. J. Plastic Surgery, 39: 554-558 (1986).
22. Clark, L. E., and Mellette, J. R., “The Use of Hyaluronidase as an Adjunct to Surgical Procedures” J. Dermatol., Surg. Oncol., 20: 842-844 (1994).
23. Nathan, N., et al., “The Role of Hyaluronidase on Lidocaine and Bupivacaine Pharmaco Kinetics After Peribulbar Blockade” Anesth Analg., 82: 1060-1064 (1996).

See also U.S. Pat. No. 6,193,963 to Stern, et al., issued Feb. 27, 2001.

Lysozyme is an enzyme capable of reducing the cementing action of ECM compounds such as chondroitin sulphate. Lysozyme (aka muramidase hydrochloride) has the advantage that it is a naturally occurring enzyme; relatively small in size (14 kD), allowing rapid movement through the ECM; and is typically well tolerated by human patients. A topical preparation of lysozyme, as a pomade of lysozyme is available (Murazyme, Asta Medica, Brazil; Murazyme, Grunenthal, Belgium, Biotene with calcium, Laclede, U.S.). The addition of lysozyme as an adjuvant to a collagen shrinkage protocol results in a reduction of the thermal transition temperature required to induce 20% collagen shrinkage by about 10-12° C. Additional background on the use of lysozyme to lower the thermal transition temperature for collagen shrinkage is available. See for instance, U.S. Pat. No. 5,484,432 to Sand, issued Jan. 16, 1996.

Those skilled in the art will recognize that a variety of adjuvants that reduce the stability of the collagen fiber, tropocollagen, and or substances that serve to cement these structures are adaptable for use with the heater implants of the invention. Adjuvant ingredients may include agents such as solvents, such as dimethylsulfoxide (DMSO), monomethylsulfoxide, polymethylsulfonate (PMSF), methylsulfonylmethane, alcohol, ethanol, ether, diethylether, and propylene glycol. Certain solvents, such as DMSO, are known to lead to the disruption of collagen fibers, and collagen turnover. When DMSO is delivered to patients with scleroderma, a condition that exhibits an overproduction of collagen and scar tissue as a symptom, an increase of excretion of hydroxyproline, a constituent of collagen, is noted. This is believed to due to increased breakdown of collagen. Solvents that will alter the hydrogen bonding interactions of collagen fibers, such as DMSO and ethanol are predicted to reduce the thermal transition temperature necessary to reach the thermal transition temperature of collagen fibers, with the reduction of thermal transition temperature being expected to be relative to the alteration of the hydrophilicity of the collagen environment by the solvent. Small diffusible solvents such as DMSO and ethanol offer the further advantage of being able to rapidly penetrate the epidermis and reach the dermis tissue, while being generally safe for use in human patients.

In a further embodiment, adjuvants may be used in combination with one another, in a manner that either further lowers the thermal transition temperature either synergistically or additively. Combining adjuvants provides a means to utilize a particular adjuvant to achieve its optimal effect, and when combined with a second adjuvant, further lower the heating necessary to achieve the desired shrinkage, while avoiding adverse side effects associated with higher doses of a particular adjuvant.

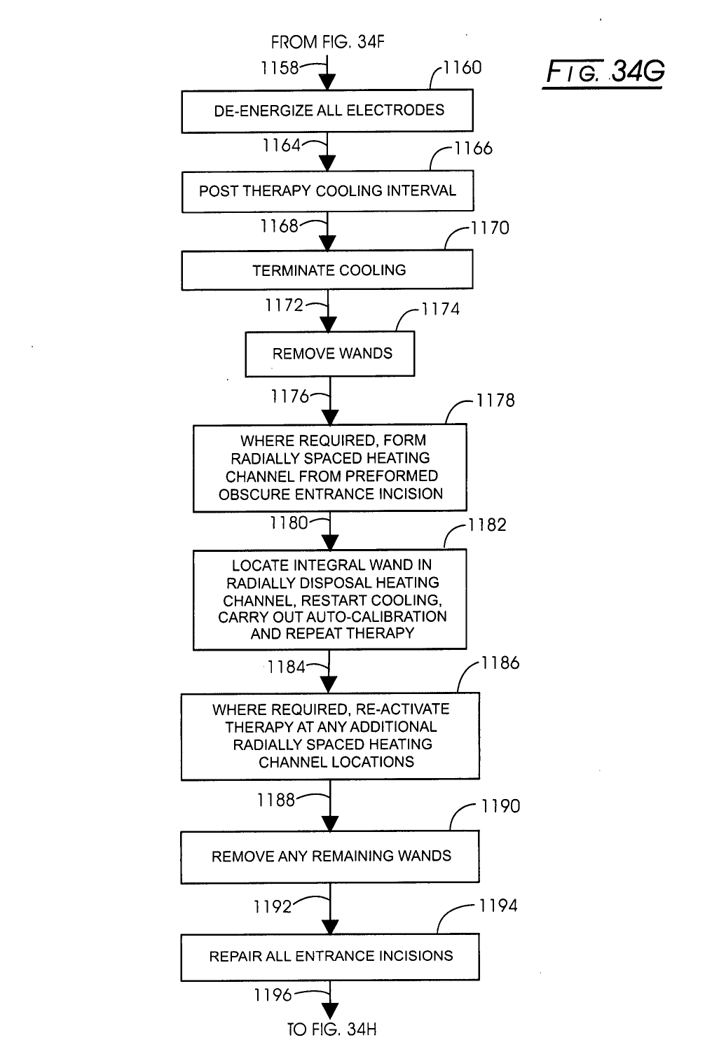

FIGS. 33A-33J combine as labeled thereon to provide a flow chart describing methodology employed with the system at hand. At the commencement of the procedure, the clinician determines that skin region suited for shrinkage as indicated at block 530. In correspondence with this determination, as represented at line 532 and block 534, a determination is made as to the desired percentage extent of linear shrinkage. In this regard, an upper limit of less than about 25% shrinkage is recommended. Next, as represented at line 536 and block 538, heating channel location or locations are determined and effective spacing is determined for bipolar R.F. electrode excitation. An entrance location is determined for each heating channel. As represented at line 540 and block 542 where the heating channels are spaced apart and in parallel relationship to receive bipolar R.F. excitable wands, the above described current path index (CPI) is computed. In this regard, reference is made to expressions (6) and (7) above. The program then continues as represented at line 544 and block 546 to determine whether the computed current path index is of an acceptably high value. In this regard, reference is again made to the data represented at FIG. 32 and the discussion associated therewith. Where the CPI value is not acceptably high, as represented at line 548 and block 550, the clinician may consider altering heating channel spacing, or as represented at line 552 and block 554, the clinician may also consider a topically applied dermis conductivity enhancing agent. The procedure then loops to line 536 as represented at line 556.

Returning to block 546 where the CPI value is acceptably high, then, as represented at line 558 and block 560, the practitioner may wish to determine heating channel location or locations with entrance locations at an obscure position, for example, behind the ear. In this regard, where energization is achievable with a single wand or implant, for example, as described in connection with FIGS. 10 and 11, then the heating channels may be developed in radially spaced fashion from a common entrance location in an obscure position, as is represented at line 562 and block 564. Where appropriate, as represented at line 566 and block 568, the procedure provides two or more wands configured with a thermal barrier supporting four electrodes and associated temperature sensing resistors. On the other hand, as represented at line 570 and block 572, a singular wand or implant as described in connection with FIGS. 10 and 11 may be employed in conjunction with a common obscure entrance location. Next, as represented at line 574 and block 576, are one or more introducer instruments for carrying out a blunt dissection of heating channels is provided. It may be recalled such an instrument has been described in connection with FIGS. 16 and 17. As represented at line 578 and block 580, the practitioner may wish to monitor skin surface temperature utilizing an IR thermographic monitor. With skin surface temperature requirements in mind, as represented at line 582 and block 584 the practitioner selects a skin surface cooling method so as to maintain the epidermis/dermis boundary below burn trauma temperature (45° C.-47° C.). As discussed above, and as represented at lines 586, 588 and block 590, a chilled airflow may be elected, whereupon the procedure continues as represented at lines 592 and 594. On the other hand, as represented at lines 586, 596 and block 598, mist airflow may be elected, for example, utilizing water or another liquid and the procedure continues as represented at lines 600, 594 whereupon as represented at block 602 a skin surface cooling approach will have been selected.

Two techniques for carrying out the R.F. electrode excitation have been described, one in connection with FIG. 19 and the other in connection with FIG. 21. Accordingly, line 610 extends to block 612 providing for an election between these two approaches to excitation. As represented at lines 614, 616 and block 618, the preferred intermittent high power on interval spaced apart by non-energization off intervals may be elected and the procedure continues as represented at line 620. On the other hand as represented at lines 614, 622 and block 624, a continuous ramp-up power modulation may be carried out to a setpoint threshold temperatures followed by a stepped-down soak interval. If that approach is elected, then the procedure continues as represented at line 626.

Returning to line 620 which extends to block 628, the practitioner is called upon to select threshold setpoint and upper limit temperatures as seen respectively at dashed lines 280 and 282 in FIG. 21. Next, as represented at line 630 and block 632, the on- and off-intervals for this intermittent excitation approach are elected. As represented at line 634 and block 636, the operator may select a ratchet-up as well as post therapy cooling intervals. The program then continues as represented at lines 638 and 640.

Returning to line 626 which extends to block 642, the operator selects threshold setpoint temperature for the continuous ramp-up excitation approach. Next, as represented at line 644 and block 646, the practitioner selects the ramp-up and soak intervals and the program proceeds as represented by lines 648, 640 and block 650 setting forth that a threshold setpoint temperature has been selected. The program then continues as represented at line 652 and block 654 determining whether an adjuvant is to be used. In the event that it is not, then the procedure continues as represented at line 656. However, in the event of an affirmative determination with respect to the query posed at block 654, then as represented at line 658 and block 660, a determination is made as to what adjuvant is to be used. With that selection, as represented at line 662 and block 664, the electrode threshold/upper limit setpoint temperatures are reduced by ΔTa. Next, as represented at line 666 and block 668, the adjuvant is administered at the skin region elected for shrinkage treatment and, as shown at line 670 and block 672, a delay ensues effective for the delivery (e.g., by diffusion) of the adjuvant into the dermis, whereupon the procedure continues as set forth at lines 674 and 656.

Line 656 extends to block 676 which, as an option, provides a starting pattern of visible indicia at the skin region of interest which is suited for evaluating a percentage of shrinkage. In this same regard, as represented at line 678 and block 680, as an option a digital image of the starting pattern may be provided. As an additional option, as represented at line 682 and block 684, a dermis conductivity enhancing agent may be topically administered. From block 684, a line 686 extends to block 688 providing for the attachment of electrode leads and resistor segment leads to the controller for purposes of carrying out a test for circuit continuity. Where that test is passed, as represented at line 690 and block 692, a conventional infiltration local anesthetic may be administered at the skin region of interest. Such an anesthetic may, for example, be lidocaine with an isotonic saline diluent.

Optionally, as represented at line 694 and block 696 to carry out a nerve block remote from the skin region of interest, such a conventional local anesthetic with isotonic saline diluent may be administered. Where some concern is present that the utilization of an electrically conductive diluent may have an adverse effect on current pathways, then, as represented at line 697 and block 698, the practitioner may optionally administer an infiltration local anesthetic agent with a low electrical conductivity biocompatible diluent. Following the administration of local anesthetic, as represented at line 700 and block 702 a delay ensues for permitting the administered anesthesia agent to become effective. Upon achieving such effectiveness, as represented at line 704 and block 706, an entrance incision is formed at each heating channel entrance location using a scalpel, such incisions permitting access to the dermis-subcutaneous fat layer interface. Following the formation of the entrance incision(s), as represented at line 708 and block 710, an introducer or dissecting instrument as above described is utilized to form a heating channel from each entrance incision. Wand insertion is represented at line 712 and block 714. In this regard, the wand may be inserted over the upwardly disposed surface of the dissecting instrument whereupon the instrument is removed and the wand remains in position within the heating channel. Alternately, the heating channel dissecting instrument may be removed and the wand inserted.

The position of insertion of the wand with respect to the location of its R.F. electrodes can be controlled by utilizing visible indicia with respect to the entrance incision as represented at line 716 and block 718. A position of the wand further can be verified as represented at line 720 and block 722 by palpation. Following such verification, as represented at line 724 and block 726, the controller associated with the cables will verify whether or not proper electrical connections have been made. In the event they have not, then as represented at line 728 and block 730, the operator will be cued as to the discrepancy and prompted to recheck connections. The program then returns to line 724 as represented at line 732. In the event of an affirmative determination to the query posed at block 726, then the procedure continues as represented at line 734 and block 736 where the operator initiates auto-calibration of all temperature sensing resistor segments with respect to setpoint temperature. Auto-calibration has been discussed above in connection with equations (2) and (3). When the setpoint temperature related resistance(s) have been developed, as set forth at line 738 and block 740, those resistance value(s) are placed in memory and the program continues as represented at line 742 and block 744. The query at block 744 determines whether auto-calibration has been successfully completed. In the event that it has not, then as represented at line 746 and block 748 the controller provides an illuminated auto-calibration fault cue and, as represented at line 750 and block 752, it provides a prompt to recheck the connections of cables and to replace any faulty implant or wand. The program then loops to line 734 as represented at line 754.

In the event of an affirmative determination with respect to the query posed at block 744, then as represented at lines 756, 758 and block 760, the anticipated ratchet-up and threshold level powering intervals are set with respect to the intermittent power approach described in connection with FIG. 21. As represented at line 762 and 764, the procedure then continues.

Where a continuous modulated power mode is to be employed as described in connection with FIG. 19, then as represented at lines 756, 766 and block 768, anticipated ramp-up and soak intervals are set and the procedure continues as represented at lines 770 and 764 to block 772 providing for the activation of skin surface cooling. Additionally, as represented at line 774 and block 776 should a skin surface temperature monitor as described in connection with block 580 be provided, then that device will be activated and the procedure continues as represented at line 778 and block 780 providing for the start or commencement of the therapy. From block 780, a line 782 extends to block 784 posing the query as to whether the skin surface temperature is excessive. In the event that skin surface temperature is excessive, then as represented at line 786 and block 788 therapy is stopped and as represented at line 790 and block 792 the operator is cued to the situation at hand. However, as represented at line 794 and block 796 surface cooling is maintained. At this juncture, the operator will need to determine the source of the problem before resuming therapy or terminating the procedure entirely.

Returning to block 784, where skin surface temperature is not excessive, then as represented at line 798 and block 800 the practitioner visually monitors the extent of shrinkage. As represented at line 802 and block 804, for a full power intermittent energization mode of performance, a determination is made as to whether an electrode has reached or exceeded the upper limit setpoint temperature, T_{USP}, as represented at dashed line 282 in FIG. 21. Where that upper limit has been reached or exceeded, then as represented at line 806 and block 788, therapy is stopped and the operator is cued as represented at line 790 and block 792. However, as represented at line 794 and block 796, surface cooling is continued and the operator will be required to determine the cause of the temperature overshoot and correct it or terminate the procedure entirely.

Where the query posed at block 804 results in a negative determination, then as represented at line 808 and block 810 the operator may observe whether or not the extent of shrinkage goal has been reached. In the event that it has not been reached, then as represented at line 812 and block 814 a determination as to whether the therapy interval has been completed is made. In the event that the interval has not been completed, then as represented at line 816 and block 818 a query is made as to whether the operator has initiated a stop therapy condition. Where the therapy has not been stopped, then as represented at line 820 the procedure reverts to line 808.

Returning to block 810, where the extent of shrinkage goal has been reached, then as represented at lines 822, 824 and block 826 all electrodes are de-energized. Similarly, where the query posed at block 814 indicates that the therapy interval is completed, then as represented at lines 828, 822, 824 and block 826, all electrodes are de-energized. Also, where the query at block 818 indicates that the operator has initiated a stop therapy condition, then as represented at lines 830, 822, 824 and block 826, all electrodes are de-energized. The procedure then continues as represented at line 832 and block 834 providing for the initiation of post therapy cooling interval timing. Even though the electrodes are de-energized, heat will be conducting to the skin surface for a short interval. Accordingly, as represented at line 836 and block 838 a query is posed as to whether this post therapy interval has been completed. In the event that it has not, then as represented by line 840 extending to line 836, the system dwells until that interval is completed. Where the determination at block 838 is that the post therapy interval is completed, then as represented at line 842 and block 844 the cooling of the skin surface is terminated and as set forth at line 846 and block 848 the practitioner may evaluate the extent of shrinkage achieved. The wand will not have been removed from heating channels. Accordingly, this shrinkage evaluation is a preliminary one. As represented at line 850 and block 852, a determination is made as to whether the extent of shrinkage is acceptable. In the event that it is not, then as represented at line 854, block 856, and line 858, skin surface cooling is reactivated and the program reverts to node A. Node A reappears in FIG. 33G in conjunction with line 860 extending to line 756 and the appropriate components of the procedure are repeated optionally with parameter adjustments.

Returning to block 852, where an acceptable extent of shrinkage is present, then as represented at line 862 and block 864, the wands are removed.

Some procedures will call for radially spaced heating channels having an entrance incision located at an obscure location. For this practice, a wand, for example, as described in connection with FIGS. 10 and 11 may be employed. Returning to FIG. 331, as represented at line 866 and block 868, where required, radially spaced heating channels are formed from an obscure entrance incision. With such formation, as represented at line 870 and block 872, an integral wand is located within a radially spaced heating channel and, as represented at line 874 and block 876, cooling and skin surface temperature monitoring is restarted. Then, as represented at line 878 and block 880, where required, this form of therapy is reiterated at any additional radially spaced heating channel locations. Following this procedure, as represented at line 882 and block 884, any remaining wands are removed and as represented at line 886 and block 888, all entrance incisions are repaired. Following such repair as represented at line 890 and block 892 the therapy is completed. In general, as represented at line 894 and block 896 the practitioner will carry out a post therapy review to identify neocollagenesis.

The implants or wands of the instant system also may be employed in treating various capillary malformations, for example, port wine stain (PWS). As discussed above in connection with Mihm, Jr., et. al, (publication 19), such lesions have been classified, for instance, utilizing video microscopy, three patterns of vascular ectasia being established; type 1 ectasia of the vertical loops of the capillary plexus; type 2 ectasia of the deeper, horizontal vessels in the capillary plexus; and type 3, mixed pattern with varying degrees of vertical and horizontal vascular ectasia. As additionally noted above, in general, due to the limited depth of laser therapy, only type 1 lesions are apt to respond to such therapy.

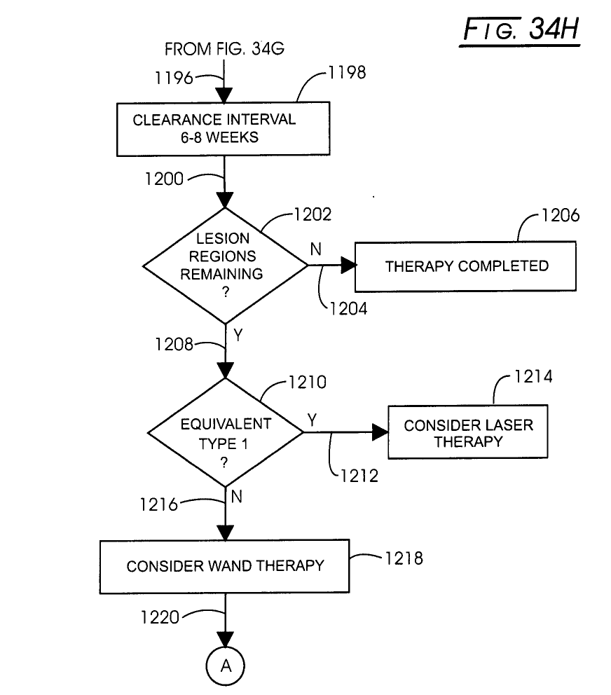

The PWS capillary malformations also are classified in accordance with their degree of vascular ectasia, four grades thereof being recognized as Grades I-IV. Such grade categorizations are discussed above. FIGS. 34A-34H combine as labeled thereon to provide a process flowchart representing an initial approach to the treatment of capillary malformation. Looking to FIG. 34A and block 910, a determination is made of the type and grade of the capillary malformation lesion. Then, as represented at line 912 and block 914, a query is posed as to whether a type 1 determination is at hand. If that is the case, then as represented at line 916 and block 918, the practitioner may wish to consider the utilization of laser therapy. On the other hand, where the determination at block 914 indicates that a type 1 lesion is not at hand, then as represented at line 920 and block 922 the practitioner will consider resort to implant therapy. For the present demonstration, a wand-based bipolar implant therapy is considered. However, a quasi-bipolar approach has been described in the above-identified application for U.S. patent Ser. No. 11/583,621 which is incorporated herein by reference. As represented at line 924 and block 926 the practitioner will select the R.F. electrode bipolar excitation method, two such methods having been described in connection with FIGS. 19 and 21. Looking initially to the approach discussed in connection with FIG. 19, lines 928 and 930 lead to block 932 describing a continuous ramp-up power modulation to a setpoint threshold temperature followed by a stepped-down power soak interval. As represented at line 934 and block 936, the practitioner will select the threshold setpoint temperature. Additionally selected are the anticipated ramp-up and soak intervals as represented at line 940 and block 942, whereupon as represented at lines 944, 946 and block 948, the bipolar electrode energization system will have been prepared.

Returning to line 928, line 950 is seen to be directed to block 952 representing an election of intermittent high power on-intervals spaced apart in time by non-energization off-intervals. This is the approach described in connection with FIG. 21. Accordingly, as represented at line 954 and block 956, the practitioner selects a threshold temperature. With the therapy at hand, a lower setpoint temperature is selected which will not adversely affect dermis tissue, i.e., that setpoint temperature will be atraumatic with respect to dermis. In general, such setpoint temperature will be in a range from about 45° C. to about 60° C. Also selected will be an upper limit temperature as described at dashed line 282 in FIG. 21. That temperature will be slightly above the selected threshold setpoint temperature. Next, as represented at line 958 and block 960, the on- and off-intervals are selected. However, they may be preprogrammed. Finally, as represented at line 962 and block 964, anticipated ratchet-up intervals and a post energization cool-down interval are selected and the procedure continues as represented at lines 966 and 946. As represented at line 968 and block 970 The practitioner determines heating channel location(s), anticipated parallel spacing for the bipolar R.F. electrode(s) and entrance location(s). Additionally as represented at line 972 and block 974, the practitioner may elect to use heating channels which radially extend from a single entrance located at an obscure position. Once the wand positional topology is determined, as represented at line 976 and block 978 current path index value (CPI) is computed for those channels which are parallel and perform in mutual bipolar relationship. Once CPI is computed, as represented at line 980 and block 982, a determination is made as to whether the computed CPI value is acceptably high. In the event that it is not, then as represented at line 984 and block 986, a CPI altering parameter such as heating channel spacing may be considered. Additionally, as represented at line 988 and block 990, the practitioner may consider enhancing the electrical conductivity of the dermis utilizing a topically applied dermis conductivity enhancing agent. The procedure then loops as represented at line 992 to line 968.

Returning to block 982, where the CPI value is acceptably high, then as represented at line 994 and block 996, there are provided two or more wands configured with a thermal barrier supporting one or more electrodes associated temperature sensing resistors. Where heating channels have been mapped in conjunction with the teachings of block 974, wands may be provided as described in conjunction with FIGS. 10 and 11. As represented at line 1004 and block 1006 These are integral wands wherein the lead assemblage is configured for effecting the R.F. energization of two or more electrodes on a common wand in bipolar fashion. Also provided, as represented at line 1008 and block 1010 are one or more introducer instruments employed for carrying out a blunt section of heating channel(s). Such an instrument has been described in connection with FIGS. 16 and 17. As an option, as represented at line 1012 and block 1014 a color I.R. thermographic skin surface temperature monitor may be utilized. As represented at line 1016 and block 1018 skin surface cooling also is called for which is required to maintain the epidermis/dermis boundary below burn trauma temperature, for instance, within a temperature range from about 45° C. to about 47° C. As represented at lines 1020, 1022 and block 1024, one approach is to cool the skin surface with a chilled airflow which, as represented at lines 1026, 1028 and block 1030 becomes the elected cooling approach. Alternately, as represented at lines 1020, 1032 and block 1034, a liquid mist airflow may be provided, such liquid, for example, being water. With that selection, as represented at lines 1036, 1028 and block 1030, the cooling approach will have been selected. Controller cables now may be coupled with the wands. Accordingly, as represented at line 1038 and block 1040, the electrode and resistor leads of each wand are coupled to the controller and are tested for circuit continuity. At this juncture, as represented at line 1042 and block 1044, the practitioner has the option of topically applying a dermis conductivity enhancing agent as discussed earlier in connection with block 990. In concert with the administration of the agent as represented at block 1044, as shown at line 1046 and block 1048, a conventional infiltration local anesthetic agent, for example, lidocaine with an isotonic saline diluent may then be administered. Optionally, as represented at line 1050 and block 1052, a nerve block removed from the skin region of interest may be administered, for example, employing a conventional lidocaine agent with isotonic saline diluent. It may be found beneficial to avoid administering an electrically conductive anesthesia agent at the skin region of interest to avoid unwanted current migration, for example, toward the subcutaneous muscle layer. As represented at line 1054 and block 1056, as a option, the practitioner may administer infiltration local anesthetic agent with low electrical conductivity biocompatible diluent. Following the administration of the agent or agents, as represented at line 1058 and block 1060, a delay ensues to permit the effectiveness of the administered agent or agents. Following such delay, as represented at line 1062 and block 1064, a scalpel is utilized to form an entrance incision at each heating channel entrance location. Then, as represented at line 1066 and block 1068, a heating channel is formed through the entrance location using an introducer instrument as provided in conjunction with block 1010. Then, as represented at line 1070 and block 1072, a wand is inserted over the outer surface of the dissecting instrument as it reposes within the heating channel. Optionally, the dissecting instrument may be removed and the wand is then inserted into the channel formed by the introducer instrument. During the procedure of forming a heating channel, the length of wand insertion can be controlled by observing the indicia located along the rearward portion of the wand as described at 112 in connection with FIG. 8. Such control is represented at line 1074 and block 1076. As set forth at lines 1078 and block 1080, the position of the wand also may be verified by palpation and following such verification, the introducer or dissecting instrument is removed. The procedure continues as represented at line 1082 to the query posed at block 1084 determining whether all cables are securely connected to the controller and to the wand leads. In the event that they are not, then as represented at line 1086 and block 1088, the practitioner is cued and prompted to recheck connections of any cables indicating fault. The procedure then loops to line 1082 as represented at line 1090. Where all cables are securely connected, as represented at line 1092 and block 1094, an auto-calibration of all temperature sensing resistors with respect to selected operating setpoint temperatures is initiated. When such resistance values have been developed, as represented at line 1096 and block 1098, the resistance value-based setpoint temperature data is placed in memory and the procedure progresses as represented at line 1100 and block 1102 to the query as to whether auto-calibration has been successfully completed. In the event it has not been so completed, then as represented at line 1104 and block 1106 an auto-calibration fault cue is published. Hence, as represented at line 1108 and block 1110, the operator is prompted to recheck connections of cables to the controller and replace any faulty wands. The program then loops to line 1092 as represented at line 1112.

Where auto-calibration has been successfully completed, then, as represented at line 1114 and block 1116, skin surface cooling is activated and, as shown at line 1118 and block 1120, where appropriate, skin surface temperature measurement is activated. With the above activations, as represented at line 1122 and block 1124, therapy is started and the procedure continues as represented at line 1126 to the query posed at block 1128 determining whether excessive skin surface temperature is at hand. In the event skin surface temperature is excessive, then as represented at line 1130 and block 1132, therapy is stopped. Line 1134 and block 1136 indicate that the operator is cued as to this stoppage. However, as set forth at line 1138 and block 1140 skin surface cooling is maintained in view of anticipated thermal inertia.

Returning to block 1128, where excessive skin surface temperature is not present, then as represented at line 1142 and block 1144, the query is made as to whether for full power intermittent energization mode of operation, has an electrode reached the upper limit setpoint temperature as described in conjunction with FIG. 21 at dashed line 282. In the event that upper limit temperature has been reached, then the procedure reverts as represented at line 1146 to line 1130 providing for a stopping of therapy, cueing of the operator and the maintenance of skin surface cooling. Where the upper limit setpoint temperature has not been reached, the procedure continues as represented at line 1148 extending to the query posed at block 1150. At block 1150, a determination is made as to whether the therapy interval has been completed. In the event that it has not, then as represented at line 1152 and block 1154 a query is posed as to whether the operator has initiated a stoppage of therapy. In the event of a negative determination, then the procedure loops to line 1148 as represented at line 1156. Returning to block 1150, where the therapy interval has been completed, then as represented at line 1158 and block 1160, all electrodes are de-energized. In similar fashion, returning to block 1154, where a stop therapy has been initiated by the operator, then as represented at lines 1162, 1158 and block 1160, all electrodes are de-energized. Not withstanding such de-energization, as represented at line 1164 and block 1166, skin surface cooling is continued for a post therapy cooling interval. Following that interval, as represented at line 1168 and block 1170, skin surface cooling is terminated whereupon as represented at line 1172 and block 1174 the wands are removed.

The practitioner may find it desirable to carry out additional therapy using integral wands as described in connection with FIGS. 10 and 11. Accordingly, as represented at line 1176 and block 1178, a heating channel which may be considered radially disposed may be formed from a pre-formed obscure entrance incision. Upon such formation, as represented at line 1180 and block 1182, an integral wand may be located within the radially disposed heating channel, skin surface cooling then is restarted, an auto-calibration of the temperature sensing resistor segments is carried out and therapy is repeated. As represented at line 1184 and block 1186, this form of therapy can be reiterated employing the common entrance incision with the formation of additional radially spaced heating channel locations. Upon the conclusion of this reiterated therapy as represented at line 1188 and block 1190, any remaining wands are removed and, as set forth at line 1192 and block 1194, all entrance incisions are repaired. As represented at line 1196 and block 1198, a clearance interval then ensues which may, for instance, be from six to eight weeks. Following that interval, as represented at line 1200 and block 1202, a determination is made as to whether there are any lesion regions remaining. If there no such lesions remaining, then therapy is completed as represented at line 1204 and block 1206. If lesions do remain, then as represented at line 1208 and block 1210, a determination is made as to whether the lesion regions remaining are equivalent to a type 1 condition. If that is the case, then as represented at line 1212 and block 1214, the practitioner may wish to consider laser therapy. Where the remaining lesion regions are not type 1, then as represented at line 1216 and block 1218, wand based therapy may be considered. If such therapy is considered appropriate, the procedure reverts to node A as represented at line 1220. Node A reappears in FIG. 34A in conjunction with line 1222 extending to line 920.

For a variety of vascular malformations, especially for instance, hemangiomas, differing treatment modalities may be appropriate. In such case, additional considerations as to the extent and invasiveness of the vascular malformation is appropriate, as discussed by Jackson, et al. (see above, eg., publications 13 and 14 and the discussions associated therewith). In such a case the procedure commences with node A as represented at line 1222. in FIG. 34A extending to line 920.

As noted above, aberrant vascular formations, including angiomas may be either proliferating or nonproliferating in character. Certain of these lesions, especially if arterially associated, may be impossible to treat by previously available therapies, because surgical resection may be dangerous. The treatment modality presented in FIG. 34 provides a mechanism for treatment of a variety of vascular malformations, including proliferating angiomas and arterial angiomas. In certain cases heating of the tissue to the temperature setpoint range of 45-65° C. is indicated, as coagulation of the targeted vascular malformation will be effective for inducing the involution of the target tissue malformation. The procedure outlined in FIG. 34 provides an advantage over present laser induced interstitial thermotherapy by providing for effective monitoring of the temperature increase induced by localized heating. Thus such deleterious side effects such as carbonization of tissue are avoided.

Alternatively, rather than heating the tissue to such as level predicted to cause irreversible cell damage and immediate death, a lower setpoint temperature may be employed at block 952 as shown in FIG. 34A. Setpoint temperatures in the range of about 40° C. to 45° C. can be expected to induce cell damage that may lead to involution of the target tissue due to induction of heat shock and or apoptosis of the target tissues. For arterial (i.e. high flow) vascular malformations, induction of apoptosis (programmed cell death) may be the only alternative for treating the vascular malformation, as the malformation may be too dangerous for surgical resection, and too deeply placed for laser treatment to be effective. Setpoint temperatures in the range of about 40° C. to 45° C. are predicted to be effective for the treatment of a variety of intransigent angiomas and hemangiomas.

Since certain changes may be made in the above apparatus and method without departing from the scope of the disclosure herein involved, it is intended that all matter contained in the above description or shown in the accompanying drawings shall be interpreted as illustrative and not in a limiting sense. All citations are hereby incorporated by reference.

1. The method for effecting a controlled heating of tissue within the region of the dermis of skin, comprising the steps:
(a) determining a skin region for treatments;
(b) providing one or more heater implants each comprising a thermally insulative generally flat support having a support surface and an oppositely disposed insulative surface, and a circuit mounted at the support surface having one or more electrodes;
(c) determining one or more heating channel locations along said skin region;
(d) locating each heater implant along a heating channel generally at the interface between dermis and next adjacent subcutaneous tissue wherein said one or more electrodes are electrically contactable with dermis and in thermally insulative relationship with said next adjacent subcutaneous tissue;
(e) effecting a radiofrequency energization of said one or more electrodes toward a threshold temperature; and
(f) simultaneously controlling the temperature of the surface of skin within said region to an extent effective to protect epidermis from thermal injury while permitting the derivation of effective therapeutic temperature at the said region of the dermis.

2. The method of claim 1 in which:
step (b) provides two or more implants; and
step (e) effects said energization in bipolar fashion.

3. The method of claim 1 in which:
step (e) is carried out to effect a controlled shrinkage of dermis or a component of dermis.

4. The method of claim 1 in which:
step (e) is carried out to effect a therapeutic treatment of a capillary malformation.

5. The method of claim 1 further comprising the step:
(g) monitoring the temperature of said one or more electrodes during step (e);

6. The method of claim 1 in which:
step (b) provides said circuit as having a polymeric substrate with an outward face supporting one or more electrodes, and an inward face supported from said support surface.

7. The method of claim 5 in which:
step (b) provides said circuit as having one or more temperature sensors each having a temperature responsive condition adjacent to said inward face in thermal exchange adjacency with a said electrode; and
step (g) carries out said monitoring of temperature by monitoring the said temperature responsive condition of each temperature sensor.

8. The method of claim 7 in which:
step (b) provides each said circuit temperature sensor as a resistor; and
step (g) carries out said monitoring of temperature in a manner wherein said temperature responsive condition is electrical resistance.

9. The method of claim 5 in which:
step (b) provides two or more implants;
step (e) effects said energization in bipolar fashion and reduces the power level to a bipolar electrode pair in response to a threshold temperature attained input; and
step (g) derives said threshold temperature attained input in correspondence with each bipolar electrode pair.

10. The method of claim 9 in which:
step (e) is carried out by progressively continuously increasing power applied to said electrode pair from an initial value toward a higher value until said threshold temperature is attained.

11. The method of claim 5 in which:
step (b) provides two or more implants having electrodes paired for bipolar energization;
step (e) effects said energization of paired electrodes at a select power level for a sequence of energization on-intervals time-spaced apart by non-energization off-intervals.

12. The method of claim 11 in which:
step (f) is carried out both during said on-intervals and off-intervals.

13. The method of claim 11 in which:
step (e) effects said energization at said select power level in bipolar fashion and reduces the power level to a bipolar electrode pair in response to a threshold temperature attained input; and
step (g) derives said threshold temperatures attained input in correspondence with each bipolar electrode pair.

14. The method of claim 11 in which:
said step (e) non-energization off-intervals exhibit a duration effective to permit step (f) to control the temperature of the surface of skin within said region to an extent effective to protect epidermis from thermal injury.

15. The method of claim 1 further comprising the step:
(h) pre-cooling said next adjacent subcutaneous tissue through the surface of skin at said skin region prior to steps (d) through (e).

16. The method of claim 1 in which:
step (f) is continued subsequent to step (e) for an interval effective to alter the temperature of heated dermis toward human body temperature.

17. The method of claim 1 in which:
step (e) is carried out to effect a therapeutic treatment of a vascular malformation.

18. The method of claim 17 in which the vascular malformation is one or more of a nonproliferative vascular malformations, a capillary malformation, a venuous malformation, a lymphatic malformation, an arterial malformation, a complex-combined vascular malformation, an angioma, and a hemangioma.

19. The method of claim 18 in which the vascular malformation is a Port Wine Stain capillary malformation.

20. The method of claim 17 in which:
step (e) is carried out to effect an irreversible vascular coagulation with a threshold temperature atraumatic to dermis.

21. The method of claim 3 further comprising the step:
(i) administering an adjuvant generally to dermis at said skin region effective to lower the thermal transition temperature for carrying out the shrinkage of dermis or a component of dermis.

22. The method of claim 21 in which:
step (i) administers said adjuvant topically at said skin region.

23. The method of claim 21 in which:
step (b) provides one or more implants as carrying said adjuvant at a location for dispersion within dermis from a heating channel.

24. The method of claim 21 in which:
the thermal transition temperature lowering adjuvant of step (i) is one or more of salt, an enzyme, a detergent, a lipophile, a denaturing solvent, an organic denaturant, and acidic solution, or a basic solution.

25. The method of claim 24 wherein the enzyme is one or more of hyaluronidase, lysozyme, muramidase, or collagenase.

26. The method of claim 24 wherein the denaturing solvent is one or more of an alcohol, an ether, monomethyl sulfoxide or DMSO.

27. The method of claim 24 wherein the organic denaturant is urea.

28. The method of claim 24 wherein two or more thermal transition temperature lowering adjuvants are present in a therapeutically effective combination.

29. The method for effecting a controlled heating based treatment of dermis located over a next adjacent subcutaneous fat layer, in turn located over next adjacent muscle tissue, comprising:
(a) determining a skin region for treatment;
(b) estimating the thickness of dermis within the skin region;
(c) estimating the thickness of the next adjacent fat layer;
(d) providing two or more implant supported electrodes;
(e) providing a current path index comparison value derived from histopathology-based evaluation of a population of tissue samples and representing a limit for avoiding traumatic radiofrequency current flow within a said next adjacent muscle tissue;
(f) estimating a current path index value based upon said estimated thickness of dermis and next adjacent fat layer and bipolar paired electrode spacing;
(g) adjusting a parameter of said treatment when the estimated current path index indicates a potential for said traumatic radiofrequency current flow;
(h) determining one or more heating channel locations for locating the two or more electrodes at a bipolar paired electrode spacing;
(i) locating each heater implant along a heating channel generally at the interface between dermis and next adjacent subcutaneous fat layer;
(j) effecting a bipolar radiofrequency energization of said electrodes toward a threshold temperature; and
(k) simultaneously controlling the temperature of the surface of skin within said region to an extent effective to protect epidermis from thermal injury while permitting the derivation of effective therapeutic temperature at said region of the dermis.

30. The method of claim 29 in which:
the step (g) adjustment of a parameter of treatment is carried out by reducing said bipolar paired electrode spacing.

31. The method of claim 29 in which:
the step (g) adjustment of a parameter of treatment is carried out by a topical administration of an agent at said skin region effective to increase the electrical conductivity of dermis.

32. The method of claim 29 in which:
step (j) effects the bipolar energization of said electrodes at a select power level for a sequence of energization on-intervals time-spaced apart by non-energization off-intervals.

33. The method of claim 32 in which:
step (j) effects said energization at said select power level and reduces the power level to a bipolar pair of electrodes in response to the attainment of a threshold temperature.

34. The method of claim 29 further comprising the step:
(l) prior to step (i) administering an adjuvant generally to dermis at said skin region effective to lower the thermal transition temperature for carrying out the shrinkage of dermis or a component of dermis.

35. The method for effecting a controlled heating of tissue within the region of the dermis of skin, comprising the steps:
(a) determining a skin region for treatment;
(b) providing two or more heater implants each comprising a thermally insulative generally flat support having a support surface and an oppositely disposed insulative surface, the support having a lengthwise dimension extending between leading and trailing ends, a widthwise dimension, a circuit mounted at the support surface having one or more electrodes;
(c) determining two or more heating channel locations at said skin region, each having a channel entrance location;
(d) forming an entrance incision at each channel entrance location;
(e) inserting a heater implant leading end through each entrance incision to locate it within a heating channel, the trailing end remaining outside the surface of said skin region, and the one or more electrodes being located for contact with adjacent dermis;
(f) applying bipolar radiofrequency energization to the one or more electrodes of the inserted implants from the trailing ends thereof for a therapy interval; and
(g) removing the implant active area through the corresponding entrance incision.

36. The method of claim 35 further comprising the step:
(h) simultaneously with step (g) controlling the temperature of the surface of skin within said skin region to an extent effective to protect the skin surface from thermal injury.

37. The method of claim 36 in which:
step (h) controls the temperature at the interface between dermis and epidermis within said region within a temperature range of from about 45° C. to about 47° C.

38. The method of claim 35 in which:
step (f) is carried out to effect a controlled shrinkage of dermis or a component of dermis.

39. The method of claim 35 in which:
step (f) is carried out to effect a therapeutic treatment of a vascular malformation.

40. The method of claim 39 in which the vascular malformation is one or more of a nonproliferative vascular malformations, a capillary malformation, a venuous malformation, a lymphatic malformation, an arterial malformation, a complex-combined vascular malformation, an angioma, and a hemangioma.

41. The method of claim 40 in which the vascular malformation is a Port Wine Stain capillary malformation.

42. The method of claim 38 further comprising the step:
(i) during and/or after step (f) and before step (g) determining an extent of skin shrinkage.

43. The method of claim 42 in which:
step (i) provides a pattern of visible indicia at said skin region prior to step (c) and visually determines the extent of relative movement of said indicia.

44. The method of claim 36 in which:
step (h) is continued subsequent to step (f) for an interval effective to alter the temperature of heated dermis toward human body temperature.

45. The method of claim 35 further comprising the step:
(j) precooling the next adjacent subcutaneous tissue to dermis through the surface of skin at said skin region prior to steps (d) through (g).

46. The method of claim 36 in which:
step (h) is carried out with a liquid containing conformal container having a contact surface located against skin at said skin region.

47. The method of claim 36 in which:
step (h) is carried out by flowing chilled air or mist containing air over said skin region.

48. The method of claim 46 in which:
step (h) is further carried out by locating a heat transferring liquid lubricant intermediate the surface of skin at said skin region and the contact surface of the container.

49. The method of claim 38 in which:
step (f) is carried out after having generally predetermined said therapy interval with respect to a desired extent of skin shrinkage and setpoint temperature.

50. The method of claim 35 further comprising the step:
(k) administering an adjuvant generally to dermis at said skin region effective to lower the thermal transition temperature for carrying out the shrinkage of dermis or a component of dermis.

51. The method of claim 50 in which:
step (b) provides one or more implants as carrying said adjuvant at a location for dispersion within dermis from the heating channel.

52. The method of claim 50 in which:
the thermal transition temperature lowering adjuvant of step (k) is one or more of salt, an enzyme, a detergent, a lipophile, a denaturing solvent, an organic denaturant, and acidic solution, or a basic solution.

53. The method of claim 50 wherein the enzyme is one or more of hyaluronidase, lysozyme, muramidase, or collagenase.

54. The method of claim 50 wherein said adjuvant is administered one or more of topically, transdermally, intradermally, subdermally, or hypodermally.

55. The method of claim 52 wherein said adjuvant is administered subdermally by release from a heater implant.

56. The method of claim 35 in which:
step (b) provides said two or more heater implants wherein said thermally insulative generally flat support lengthwise dimension is a fixed, consistent value, and said circuit has a fixed, consistent number of electrodes having a common length which may vary among given implants.

57. The method of claim 56 in which:
step (b) provides said two or more implants as having a flat support exhibiting a lengthwise dimension of about 7.5 inches.

58. The method of claim 35 in which:
step (b) provides said two or more implants with one or more electrodes formed of a metal having a thickness effective to promote the spreading dispersion of thermal energy into the region of dermis.

59. The method of claim 35 in which:
step (b) provides said two or more implants with one or more electrodes formed with copper having a thickness of between about 0.005 inch and about 0.020 inch.

60. The method of claim 39 in which:
step (f) is carried out to effect an irreversible vascular coagulation with a setpoint temperature and therapy interval atraumatic to dermis.

61. The method of claim 60 in which:
step (f) is carried out with a setpoint temperature within the range from about 45° C. to about 60° C.

62. The method of claim 60 in which:
step (f) is carried out with a setpoint temperature within the range from about 40° C. to about 45° C.

63. The method for effecting a controlled heating of a capillary malformation within a skin region comprising the steps:
(a) determining the degree of vascular ectasia at said region;
(b) providing one or more heater implants each comprising a thermally insulative generally flat support having a support surface and an oppositely disposed insulative surface, the support having an active length, a circuit mounted at the support surface having one or more electrodes along the active length;
(c) determining one or more heating channel locations within said region each having an entrance location;
(d) locating each heater implant along a heating channel generally at the interface between dermis and next adjacent subcutaneous tissue in an orientation wherein said one or more electrodes are electrically contactible with dermis and in thermally insulative relationship with said next adjacent subcutaneous tissue;
(e) simultaneously controlling the temperature of the surface of skin within said region to an extent effective to protect the skin surface from thermal injury while permitting the derivation of effective therapeutic temperature at the said skin region dermis; and
(f) effecting a radiofrequency energization of said electrodes heating them toward a setpoint temperature atraumatic to dermis while effecting an irreversible vascular coagulation at the skin region.

64. The method of claim 63 in which:
step (f) effects said energization of said electrodes toward a setpoint temperature within a range of between about 45° C. and about 60° C.

65. The method of claim 63 in which:
step (f) effects said energization of said electrodes toward a setpoint temperature within a range of between about 40° C. and about 45° C.

66. The method of claim 63 furthering comprising the step:
(g) monitoring the temperature of each said electrode during step (f).

67. The method of claim 66 in which:
step (b) provides said implants as having one or more temperature sensors, each having a temperature responsive condition corresponding with the temperature of an electrode; and
step (g) carries out the monitoring of temperature by monitoring said temperature responsive condition.

68. The method of claim 63 in which:
step (e) is carried out by flowing chilled air or mist containing air over said skin region.

69. The method of claim 63 in which:
step (e) is carried out with a conformal polymeric container having a contact surface located against skin at said skin region.

70. The method of claim 63 in which:
step (b) provides two or more implants; and
step (g) effects said energization in bipolar fashion.

71. The method of claim 63 further comprising the steps:
(j) subsequent to step (f) removing said one or more implants from each heating channel;
(k) waiting a clearance interval at least effective for the resorption of tissue at said skin region which has undergone irreversible vascular coagulation; and
(l) then repeating step (a).

72. The method of claim 71 further comprising the steps:
(m) where step (l) determines that any remaining capillary malformation is equivalent to a type 1 lesion, treating the remaining capillary malformation using laser-based therapy.

73. The method for effecting a heating of tissue within the region of the dermis of skin, comprising the steps:
(a) determining a skin region for treatment;
(b) providing one or more implants each having one or more R.F. excitable electrodes;
(c) determining one or more heating channel locations along said skin region;
(d) locating each heater implant along a heating channel generally at the interface between dermis and next adjacent subcutaneous tissue wherein said one or more electrodes are contactable with dermis;
(e) selecting a temperature threshold level for said one or more electrodes;
(f) effecting radiofrequency power energization of said one or more electrodes wherein said energization is carried out during power-on intervals spaced apart in time by power-off intervals at least to substantially maintain said temperature threshold level; and
(g) simultaneously controlling the temperature of the surface of skin within said region to an extent effective to protect epidermis from thermal injury while permitting the derivation of effective treatment temperature at the said region of the dermis.

74. The method of claim 73 in which:
step (f) substantially maintains said temperature threshold by selectively curtailing said radiofrequency power energization in response to an electrode reaching said temperature threshold.

75. The method of claim 73 in which:
said step (f) power-off intervals exhibit a duration effective to permit step (g) to control the temperature of the surface of skin within said region to an extent effective to protect epidermis from thermal injury.

76. The method of claim 73 in which:
step (e) further selects a temperature upper limit level; and
step (f) terminates said power energization in response to an electrode reaching a temperature at said upper limit level.

77. The method of claim 73 in which:
step (g) is continued subsequent to step (f) for an interval effective to alter the temperature of heated dermis toward human body temperature.

78. The method of claim 73 in which:
steps (e) and (f) are carried out to effect therapeutic treatment of a vascular malformation.

79. The method of claim 78 wherein the vascular malformation is one or more of a nonproliferative vascular malformations, a capillary malformation, a venuous malformation, a lymphatic malformation, an arterial malformation, a complex-combined vascular malformation, an angioma, and a hemangioma.

80. The method of claim 79 wherein the vascular malformation is a capillary malformation.

81. The method of claim 73 further comprising the step:
(h) administering an adjuvant generally to dermis at said skin region effective to lower the thermal transition temperature for carrying out the shrinkage of dermis or a component of dermis.

82. The method of claim 81 in which:
the thermal transition temperature lowering adjuvant of step (h) is one or more of salt, an enzyme, a detergent, a lipophile, a denaturing solvent, an organic denaturant, and acidic solution, or a basic solution.

83. The method of claim 82 wherein the enzyme is one or more of hyaluronidase, lysozyme, muramidase, or collagenase.

84. The method of claim 82 wherein the denaturing solvent is one or more of an alcohol, an ether, monomethyl sulfoxide or DMSO.

85. The method of claim 82 wherein the organic denaturant is urea.

86. The method of claim 82 wherein two or more thermal transition temperature lowering adjuvants are present in a therapeutically effective combination.
